(19) United States
(12) Patent Application Publication
(10) Pub. No.: US 2017/0183408 A1
(43) Pub. Date: Jun. 29, 2017

(54) ANTIBODY SCAFFOLD FOR HOMOGENOUS CONJUGATION

(71) Applicant: MedImmune, LLC, Gaithersburg, MD (US)

(72) Inventor: Nazzareno Dimasi, Gaithersburg, MD (US)

(21) Appl. No.: 15/384,908

(22) Filed: Dec. 20, 2016

Related U.S. Application Data
(63) Continuation of application No. 13/883,972, filed on Aug. 26, 2013, now abandoned.
(60) Provisional application No. 61/411,588, filed on Nov. 9, 2010.

(51) Int. Cl. C07K 16/28; A61K 49/00

(52) U.S. Cl. CPC C07K 16/283; A61K 49/00; C07K 2317/56; C07K 2317/92

(57) ABSTRACT

Provided in some embodiments are antibodies comprising a heavy chain having no native interchain cysteine amino acids, a light chain having no native interchain cysteine amino acids, and having no native interchain disulphide linkages between the heavy chain and the light chain. Also provided in certain embodiments are antibodies comprising a heavy chain having no native interchain cysteine amino acids, a light chain having no native interchain cysteine amino acids, and having no native interchain disulphide linkages between the heavy chain and the light chain where the native interchain cysteine amino acids have been replaced by amino acids having no thiol moiety.

### SEQUENCE LISTING

The instant application contains a Sequence Listing which has been submitted in ASCII format via EFS-Web and is hereby incorporated by reference in its entirety. Said ASCII copy, created on Nov. 7, 2011, is named MED0595P.txt and is 34,826 bytes in size.

### FIELD

The technology relates in part to engineered antibodies and antibody conjugates. Such antibodies and conjugates can be utilized for diagnostic and therapeutic applications in some embodiments.

### BACKGROUND

Antibodies, which also are referred to as immunoglobulins (Ig) are proteins that naturally occur in blood or other bodily fluids of vertebrates. Antibodies are immune system agents that bind to and neutralize foreign objects, such as bacteria and viruses.

Naturally occurring antibodies generally include two larger heavy chains and two smaller light chains. In the case of native full-length antibodies, these chains join together to form a “Y-shaped” protein (e.g., FIG. 1A). Heavy chains and light chains include cysteine amino acids that can be joined to one another via disulfide linkages. Heavy chains are joined to one another in an antibody by disulfide linkages between cysteine amino acids in each chain. Light chains are joined to heavy chains also by disulfide linkages between cysteine amino acids in the chains. Such disulfide linkages generally are formed between thiol side chain moieties of the free cysteine amino acids. Three particular cysteine amino acids in each heavy chain and one particular cysteine in each light chain sometimes are referred to as “interchain cysteines” as they generally participate in disulfide linkages between antibody chains.

An antibody may be conjugated to another molecule, which can be useful for diagnostic applications (e.g., the conjugated molecule can be a detectable label) or therapeutic applications (e.g., the conjugated molecule can be a toxin). The molecule can be conjugated to the antibody by a linkage with a cysteine amino acid of the antibody in certain instances.

### SUMMARY

Provided herein, in some embodiments, is an antibody that includes a heavy chain having no native interchain cysteine amino acids, a light chain having no native interchain cysteine amino acids, and no native interchain disulphide linkages between the heavy chain and the light chain. In certain embodiments the antibody comprises no interchain disulphide linkages between the heavy chain and the light chain under conditions that native antibodies could form interchain disulphide linkages between the chains. In some embodiments, provided is an antibody, comprising a heavy chain having no interchain cysteine amino acids; a light chain having no interchain cysteine amino acids; and no interchain disulphide linkages between the heavy chain and the light chain.

In various embodiments the antibody comprises two heavy chains and two light chains. The antibody is sometimes a full-length antibody. In some embodiments the heavy chain is about 400 to about 500 amino acids in length (e.g., about 420, 440, 460, 480 amino acids; about 446 amino acids in length) and the light chain is about 200 to about 300 amino acids in length (e.g., about 220, 240, 260, 280 amino acids; about 214 amino acids in length). In certain embodiments the antibody is a human antibody. In various embodiments the antibody is a humanized antibody.

In some embodiments, provided is an antibody that comprises: (i) a IgG1, IgG2, IgG3 or IgG4 isotype heavy chain constant region lacking interchain cysteines, and (ii) a kappa or lambda light chain constant region lacking an interchain cysteine. In certain embodiments, interchain cysteine amino acids are substituted by a non-cysteine amino acid (e.g., an amino acid that does not contain a thiol moiety) in antibodies that lack interchain cysteines. In some embodiments, provided is a fragment of a full-length antibody that comprises (i) a portion of an IgG1, IgG2, IgG3 and IgG4 isotype heavy chain constant region lacking interchain cysteines, and optionally (ii) a kappa or lambda light chain constant region, or portion thereof, lacking an interchain cysteine. In certain embodiments, an antibody comprises an Fc region or fragment thereof. Examples of IgG1, IgG2, IgG3 or IgG4 isotype heavy chain constant region amino acid sequences are shown in SEQ ID NOs: 5, 6, 7 and 8, respectively, and examples of kappa and lambda light chain constant region amino acid sequences are shown in SEQ ID NOs: 9 and 10, respectively, hereafter.

>IgG1
(SEQ ID NO: 5)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
>IgG2
(SEQ ID NO: 6)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK
>IgG3
(SEQ ID NO: 7)
ASTKGPSVFPLAPCSRSTSGGTAALGCLVKDYFPEPVTVSWNSGALTSGV
HTFPAVLQSSGLYSLSSVVTVPSSSLGTQTYTCNVNHKPSNTKVDKRVEL
KTPLGDTTHTCPRCPEPKSCDTPPPCPRCPEPKSCDTPPPCPRCPEPKSC
DTPPPCPRCPAPELLGGPSVFLFPPKPKDTLMISRTPEVTCVVVDVSHED
PEVQFNWYVDGVEVHNAKTKPREEQYNSTYRVVSVLTVLHQDWLNGKEYK
CKVSNKALPAPIEKTISKTKGQPREPQVYTLPPSREEMTKNQVSLTCLVK
GFYPSDIAVEWESSGQPENNYKTTPPVLDSDGSFFLYSKLTVDKSRWQQG
NIFSCSVMHEALHNHFTQKSLSLSPGK
>IgG4
(SEQ ID NO: 8)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
>kappa
(SEQ ID NO: 9)
VAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSF
NRGEC
>lambda
(SEQ ID NO: 10)
KAAPSVTLFPPSSEELQANKATLVCLISDFYPGAVTVAWKADSSPVKAGV
ETTTPSKQSNNKYAASSYLSLTPEQWKSHRSYSCQVTHEGSTVEKTVAPT
ECS

Antibodies can be engineered to lack interchain cysteines in a variety of manners. In certain antibodies, one or more or all interchain cysteines independently can be substituted with an amino acid that is not cysteine. An amino acid substituted for an interchain cysteine often does not include a thiol moiety, and sometimes is a serine, threonine, valine, alanine, glycine, leucine or isoleucine, other naturally occurring amino acid, or non-naturally occurring amino acid. Interchain cysteines that replaced by another non-cysteine amino acid sometimes are replaced by the same amino acid or by one or more different amino acids. In some embodiments, one or more or all interchain cysteines can be deleted and not replaced by another amino acid.

The following Table 1 illustrates positions of interchain cysteines in the heavy chain constant region and light chain constant region of particular antibody isotypes with reference to Kabat EU numbering and with reference to the sequences disclosed herein. Each or the interchain cysteine positions present in an antibody or antibody fragment often are deleted or substituted with an amino acid that is not a cysteine.

TABLE 1
Native Interchain Cysteine Positions in Antibody Constant Regions

| Antibody | | | | | | | |
|---|---|---|---|---|---|---|---|
| Isotype | Kabat EU/SEQ ID NO | Position of Cysteine | | | | | |
| Heavy | Kabat EU position | 131 | 220 | N/A | N/A | 226 | 229 |
| chain | | | | | | | |
| IgG1 | Corresponding position in SEQ ID NO: 5 | N/A | 103 | N/A | N/A | 109 | 112 |
| IgG2 | Corresponding position in SEQ ID NO: 6 | 14 | 103 | N/A | N/A | 106 | 109 |
| IgG3 | Corresponding position in SEQ ID NO: 7 | 14 | N/A | 110 | 113 | 118 | 121 |
| IgG4 | Corresponding position in SEQ ID NO: 8 | 14 | N/A | N/A | N/A | 106 | 109 |

| | | |
|---|---|---|
| Light chain | | |
| Kappa | Kabat EU Position | 214 |
| | Corresponding position in SEQ ID NO: 9 | 105 |
| Lambda | Kabat EU Position | 213 |
| | Corresponding position in SEQ ID NO: 10 | 102 |

The location of corresponding interchain cystein positions in the IgG isotypes may also be identified by reference to FIG. 22.

Thus, in some embodiments, provided is an antibody comprising a heavy chain comprising the amino acid sequence of SEQ ID NO: 5, and a light chain comprising the amino acid sequence of SEQ ID NO: 9 or SEQ ID NO: 10, where each of the cysteines at positions 103, 109, and 112 in SEQ ID NO: 5, and the cysteine at position 105 in SEQ ID NO: 9 or the cysteine at position 102 in SEQ ID NO: 10, are substituted by an amino acid that is not cysteine. The antibody often comprises no interchain cysteine amino acids and often comprises no interchain disulfide linkages.

Provided also in certain embodiments is an antibody comprising a heavy chain comprising the amino acid sequence of SEQ ID NO: 5, where each of the cysteines at positions 103, 109, and 112 in SEQ ID NO: 5 is substituted by an amino acid that is not cysteine, and where the antibody often comprises no interchain cysteine amino acids and often includes no interchain disulfide linkages.

Also provided in some embodiments is an antibody comprising a heavy chain fragment comprising a portion of the amino acid sequence of SEQ ID NO: 5, provided that should the fragment include an amino acid at position(s) 103, 109, and/or 112 in SEQ ID NO: 5, the amino acid at each of the positions is not a cysteine, and where the antibody often comprises no interchain cysteine amino acids and often includes no interchain disulfide linkages. In some embodiments, such antibodies comprise a light chain comprising the amino acid sequence of SEQ ID NO: 9 or SEQ ID NO: 10, where the cysteine at position 105 in SEQ ID NO: 9 or the cysteine at position 102 in SEQ ID NO: 10 is substituted by an amino acid that is not cysteine. In certain embodiments, such antibodies comprise a light chain fragment comprising a portion of the amino acid sequence of SEQ ID NO: 9 or SEQ ID NO: 10, provided that should the fragment include position 105 of SEQ ID NO: 9 or position 102 of SEQ ID NO: 10, the amino acid at that position is not a cysteine. In some embodiments, an antibody comprises an amino acid sequence comprising 80% or more amino acid sequence identity (e.g., about 85% or more, 86% or more, 87% or more, 88% or more, 89% or more, 90% or more, 91% or more, 92% or more, 93% or more, 94% or more, 95% or more, 96% or more, 97% or more, 98% or more, 99% or more sequence identity) to an antibody described in this paragraph or the foregoing paragraph, where the antibody often comprises no interchain cysteine amino acids and often comprises no interchain disulfide linkages.

In certain embodiments, provided is an antibody comprising a heavy chain comprising the amino acid sequence of SEQ ID NO: 6, and a light chain comprising the amino acid sequence of SEQ ID NO: 9 or SEQ ID NO: 10, where each of the cysteines at positions 14, 103, 106, and 109 in SEQ ID NO: 6, and the cysteine at position 105 in SEQ ID NO: 9 or the cysteine at position 102 in SEQ ID NO: 10, are substituted by an amino acid that is not cysteine. The antibody often comprises no interchain cysteine amino acids and often comprises no interchain disulfide linkages.

Also provided in some embodiments is an antibody comprising a heavy chain comprising the amino acid sequence of SEQ ID NO: 6, where each of the cysteines at positions 14, 103, 106, and 109 in SEQ ID NO: 6 is substituted by an amino acid that is not cysteine, and where the antibody often comprises no interchain cysteine amino acids and often includes no interchain disulfide linkages. Provided also in certain embodiments is an antibody comprising a heavy chain fragment comprising a portion of the amino acid sequence of SEQ ID NO: 6, provided that should the fragment include an amino acid at position(s) 14, 103, 106, and/or 109 in SEQ ID NO: 6, the amino acid at each of the positions is not a cysteine, and where the antibody often comprises no interchain cysteine amino acids and often includes no interchain disulfide linkages. In some embodiments, such antibodies comprise a light chain comprising the amino acid sequence of SEQ ID NO: 9 or SEQ ID NO: 10, where the cysteine at position 105 in SEQ ID NO: 9 or the cysteine at position 102 in SEQ ID NO: 10 is substituted by an amino acid that is not cysteine. Such antibodies, in certain embodiments, can comprise a light chain fragment comprising a portion of the amino acid sequence of SEQ ID NO: 9 or SEQ ID NO: 10, provided that should the fragment include position 105 of SEQ ID NO: 9 or position 102 of SEQ ID NO: 10, the amino acid at that position is not a cysteine. In some embodiments, an antibody comprises an amino acid sequence comprising 80% or more amino acid sequence identity (e.g., about 85% or more, 86% or more, 87% or more, 88% or more, 89% or more, 90% or more, 91% or more, 92% or more, 93% or more, 94% or more, 95% or more, 96% or more, 97% or more, 98% or more, 99% or more sequence identity) to an antibody described in this paragraph or the foregoing paragraph, where the antibody often comprises no interchain cysteine amino acids and often comprises no interchain disulfide linkages.

In some embodiments, provided is an antibody comprising a heavy chain comprising the amino acid sequence of SEQ ID NO: 7, and a light chain comprising the amino acid sequence of SEQ ID NO: 9 or SEQ ID NO: 10, where each of the cysteines at positions 14, 110, 113, 118 and 121 in SEQ ID NO: 7, and the cysteine at position 105 in SEQ ID NO: 9 or the cysteine at position 102 in SEQ ID NO: 10, are substituted by an amino acid that is not cysteine. The antibody often comprises no interchain cysteine amino acids and often comprises no interchain disulfide linkages.

Also provided in certain embodiments is an antibody comprising a heavy chain comprising the amino acid sequence of SEQ ID NO: 7, where each of the cysteines at positions 14, 110, 113, 118 and 121 in SEQ ID NO: 7 is substituted by an amino acid that is not cysteine, and where the antibody often comprises no interchain cysteine amino acids and often includes no interchain disulfide linkages. In some embodiments, provided is an antibody comprising a heavy chain fragment comprising a portion of the amino acid sequence of SEQ ID NO: 7, provided that should the fragment include an amino acid at position(s) 14, 110, 113, 118 and/or 121 in SEQ ID NO: 7, the amino acid at each of the positions is not a cysteine, and where the antibody often comprises no interchain cysteine amino acids and often includes no interchain disulfide linkages. Such antibodies, in some embodiments, comprise a light chain comprising the amino acid sequence of SEQ ID NO: 9 or SEQ ID NO: 10, where the cysteine at position 105 in SEQ ID NO: 9 or the cysteine at position 102 in SEQ ID NO: 10 is substituted by an amino acid that is not cysteine. Such antibodies, in certain embodiments, comprise a light chain fragment comprising a portion of the amino acid sequence of SEQ ID NO: 9 or SEQ ID NO: 10, provided that should the fragment include position 105 of SEQ ID NO: 9 or position 102 of SEQ ID NO: 10, the amino acid at that position is not a cysteine. In some embodiments, an antibody comprises an amino acid sequence comprising 80% or more amino acid sequence identity (e.g., about 85% or more, 86% or more, 87% or more, 88% or more, 89% or more, 90% or more, 91% or more, 92% or more, 93% or more, 94% or more, 95% or more, 96% or more, 97% or more, 98% or more, 99% or more sequence identity) to an antibody described in this paragraph or the foregoing paragraph, where the antibody often comprises no interchain cysteine amino acids and often comprises no interchain disulfide linkages.

In some embodiments, provided is an antibody comprising a heavy chain comprising the amino acid sequence of SEQ ID NO: 8, and a light chain comprising the amino acid sequence of SEQ ID NO: 9 or SEQ ID NO: 10, where each of the cysteines at positions 14, 106 and 109 in SEQ ID NO: 8, and the cysteine at position 105 in SEQ ID NO: 9 or the cysteine at position 102 in SEQ ID NO: 10, are substituted by an amino acid that is not cysteine. The antibody often comprises no interchain cysteine amino acids and often comprises no interchain disulfide linkages.

Provided in certain embodiments is an antibody comprising a heavy chain comprising the amino acid sequence of SEQ ID NO: 8, where each of the cysteines at positions 14, 106 and 109 in SEQ ID NO: 8 is substituted by an amino acid that is not cysteine, and where the antibody often comprises no interchain cysteine amino acids and often comprises no interchain disulfide linkages. Provided also in some embodiments is an antibody comprising a heavy chain fragment comprising a portion of the amino acid sequence of SEQ ID NO: 8, provided that should the fragment include an amino acid at position(s) 14, 106 and/or 109 in SEQ ID NO: 8, the amino acid at each of the positions is not a cysteine, and where the antibody often comprises no interchain cysteine amino acids and often includes no interchain disulfide linkages. Such antibodies, in some embodiments, comprise a light chain comprising the amino acid sequence of SEQ ID NO: 9 or SEQ ID NO: 10, where the cysteine at position 105 in SEQ ID NO: 9 or the cysteine at position 102 in SEQ ID NO: 10 is substituted by an amino acid that is not cysteine. Such antibodies, in certain embodiments, comprise a light chain fragment comprising a portion of the amino acid sequence of SEQ ID NO: 9 or SEQ ID NO: 10, provided that should the fragment include position 105 of SEQ ID NO: 9 or position 102 of SEQ ID NO: 10, the amino acid at that position is not a cysteine. In some embodiments, an antibody comprises an amino acid sequence comprising 80% or more amino acid sequence identity (e.g., about 85% or more, 86% or more, 87% or more, 88% or more, 89% or more, 90% or more, 91% or more, 92% or more, 93% or more, 94% or more, 95% or more, 96% or more, 97% or more, 98% or more, 99% or more sequence identity) to an antibody described in this paragraph or the foregoing paragraph, where the antibody often comprises no interchain cysteine amino acids and often comprises no interchain disulfide linkages.

In certain embodiments, the amino acid sequence of the light chain in an antibody lacking interchain cysteines is about 80% or more identical to the amino acid sequence of SEQ ID NO: 3 (e.g., about 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% identical to the amino acid sequence of SEQ ID NO: 3). In some embodiments, the amino acid sequence of the light chain is identical to the amino acid sequence in SEQ ID NO: 3 except that it includes 1, 2, 3, 4, 5, 6, 7, 8, 9 or 10 amino acid modifications (e.g., substitutions, insertions and/or deletions) relative to the amino acid sequence in SEQ ID NO: 3. In some embodiments, the amino acid sequence of the heavy chain is about 80% or more identical to the amino acid sequence of SEQ ID NO: 4 (e.g., about 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% identical to the amino acid sequence of SEQ ID NO: 4). In some embodiments, the amino acid sequence of the heavy chain is identical to the amino acid sequence in SEQ ID NO: 4 except that it includes 1, 2, 3, 4, 5, 6, 7, 8, 9 or 10 amino acid modifications (e.g., substitutions, insertions and/or deletions) relative to the amino acid sequence in SEQ ID NO: 4.

Provided also, in some embodiments, is an antibody lacking interchain cysteines that includes a glycosylation site not present in an antibody counterpart having a native interchain cysteine amino acid. In some embodiments, an antibody lacking native interchain cysteines lacks one or more glycosylation moieties present in a counterpart antibody having interchain cysteine moieties, and in some embodiments, an antibody is aglycosylated. In certain embodiments, an antibody lacking native interchain cysteine amino acids includes one or more engineered glycosylation sites (e.g., one or more non-native glycosylation sites can be engineered into an antibody herein).

In various embodiments, an antibody lacking native interchain cysteines comprises one or more cysteine replacements of non-cysteine surface amino acids in the CH1 domain, CH2 domain, or CH3 domain, or combination thereof, of the antibody, when the CH1 domain, CH2 domain and/or CH3 domain, or portion thereof, is present in the antibody. The antibody sometimes comprises about 2 to about 40 of the cysteine replacements of the non-cysteine surface amino acids. In some embodiments the antibody comprises about 2 to about 40 free thiols. The antibody is a human antibody in certain embodiments.

The following Table 2 shows certain amino acids located in a loop of the CH1 domain of a heavy chain constant region of an antibody, one or more of which can be substituted by a cysteine amino acid in an antibody lacking native interchain cysteines.

TABLE 2
CH1 Loop Amino Acids

| Antibody | Kabat EU Position and Corresponding Amino Acid | | | | | | | | | SEQ ID |
|---|---|---|---|---|---|---|---|---|---|---|
| Isotype | 131 | 132 | 133 | 134 | 135 | 136 | 137 | 138 | 139 | NO: |
| IgG1 | Ser | Ser | Lys | Ser | Thr | Ser | Gly | Gly | Thr | 5 |
| IgG2 | Cys | Ser | Arg | Ser | Thr | Ser | Glu | Ser | Thr | 6 |
| IgG3 | Cys | Ser | Arg | Ser | Thr | Ser | Gly | Gly | Thr | 7 |
| IgG4 | Cys | Ser | Arg | Ser | Thr | Ser | Glu | Ser | Thr | 8 |

In some embodiments, 1, 2, 3, 4, 5, 6, 7, 8 or 9 of the amino acids shown in Table 2 independently are substituted by a cysteine amino acid. Thus, in some embodiments, one or more non-cysteine amino acids at positions 131, 132, 133, 134, 135, 136, 137, 138 and 139 in the loop shown in Table 2 independently may be substituted with a cysteine amino acid in an IgG1, IgG2, IgG3, or IgG4 heavy chain or fragment thereof. In certain embodiments, Ser 131 of an IgG1 molecule or fragment is substituted with cysteine in an IgG1 antibody or counterpart position in an IgG2, IgG3 or IgG4 antibody. The one or more cysteine replacements sometimes independently are at one or more of positions 135 and 139 of an IgG1 antibody or counterpart position in an IgG2, IgG3 or IgG4 antibody, or fragment of an antibody. In some embodiments, one or more non-cysteine amino acids at positions 131, 132, 134, 135, 136, and 139 in the loop shown in Table 2 independently may be substituted with a cysteine amino acid in an IgG1, IgG2, IgG3, or IgG4 heavy chain or fragment thereof.

The following Table 3 shows certain surface amino acids located in CH2 and CH3 domains of a heavy chain constant region of an antibody, one or more of which may be substituted by a cysteine amino acid in an antibody lacking native interchain cysteines.

TABLE 3
CH2 and CH3 Positions that may be Substituted with Cysteine

| | | | | | | | | | | | | | | | | | | | | | SEQ |
|---|---|---|---|---|---|---|---|---|---|---|---|---|---|---|---|---|---|---|---|---|---|
| Antibody | Position (Kabat EU) and Corresponding Amino Acid | | | | | | | | | | | | | | | | | | | | ID |
| Isotype | 239 | 282 | 289 | 297 | 312 | 324 | 330 | 335 | 337 | 339 | 356 | 359 | 361 | 383 | 384 | 398 | 400 | 422 | 440 | 442 | No: |
| IgG1 | Ser | Val | Thr | Asn | Asp | Ser | Ala | Thr | Ser | Ala | Glu | Thr | Asn | Ser | Asn | Leu | Ser | Val | Ser | Ser | 5 |
| IgG2 | Ser | Val | Thr | Asn | Asp | Ser | Ala | Thr | Ser | Thr | Glu | Thr | Asn | Ser | Asn | Leu | Ser | Val | Ser | Ser | 6 |
| IgG3 | Ser | Val | Thr | Asn | Asp | Ser | Ala | Thr | Ser | Thr | Glu | Thr | Asn | Ser | Ser | Leu | Ser | Ile | Ser | Ser | 7 |
| IgG4 | Ser | Val | Thr | Asn | Asp | Ser | Ser | Thr | Ser | Ala | Glu | Thr | Asn | Ser | Asn | Leu | Ser | Val | Ser | Ser | 8 |

In some embodiments of an antibody lacking native interchain cysteines, one or more of the following positions independently may be replaced with a cysteine amino acid: 239, 282, 289, 297, 312, 324, 330, 335, 337, 339, 356, 359, 361, 383, 384, 398, 400, 422, 440, and 442 of an IgG1 antibody or counterpart position in an IgG2, IgG3 or IgG4 antibody. Thus, in some embodiments, one or more non-cysteine amino acids at positions 239, 282, 289, 297, 312, 324, 330, 335, 337, 339, 356, 359, 361, 383, 384, 398, 400, 422, 440, shown in Table 3 independently may be substituted with a cysteine amino acid in an IgG1, IgG2, IgG3 or IgG4 heavy chain or fragment thereof.

In certain embodiments, an antibody lacking interchain cysteines has a stability of about 70% or more compared to an antibody counterpart containing all native interchain cysteines (e.g., about 75% or more, 80% or more, 85% or more, 90% or more, 95% or more of the stability). In some embodiments the stability is in vitro stability, and sometimes, the stability is in serum at about 37 degrees Celsius for 5 days or more. The stability is determined by calorimetry in certain embodiments. In some embodiments, the stability is in vivo stability, and sometimes, the stability is in an animal for 14 days or more. In some embodiments, an antibody lacking native interchain cysteines can include a YTE modification, as described herein.

An antibody lacking interchain cysteines sometimes has a specific binding activity of about 70% or more compared to an antibody counterpart containing all native interchain cysteines (e.g., about 75% or more, 80% or more, 85% or more, 90% or more, 95% or more of the binding activity). In certain embodiments, the specific binding activity is in vitro. The specific binding activity sometimes is quantified by an in vitro homogeneous assay or an in vitro heterogeneous assay. In some embodiments the specific binding activity is in vivo, and sometimes, the specific binding activity is determined in situ.

Certain antibodies can inhibit cell proliferation activity. In certain embodiments, an antibody lacking interchain cysteines has a cell proliferation inhibition activity of about 70% or more compared to an antibody counterpart containing all native interchain cysteines (e.g., about 75% or more, 80% or more, 85% or more, 90% or more, 95% or more of the cell proliferation inhibition). In some embodiments, the cell proliferation inhibition activity is inhibition of cancer cell proliferation. In various embodiments, the activity is in vitro, and sometimes, the activity is in vivo.

In some embodiments, the antibody of any one of the foregoing embodiments is about 90% or more monomeric (e.g., about 95% or more monomeric). The antibody of may be 90% or more monomeric in vitro.

Provided also, in various embodiments, is the antibody of any one of the foregoing embodiments which does not bind detectably to a human leukocyte receptor. The human leukocyte receptor is sometimes a Fc gamma RIII receptor.

Also provided, in some embodiments, is the antibody of any one of the foregoing embodiments, which binds to a neonatal Fc receptor with about 80% or more of the binding affinity compared to an antibody counterpart containing all native interchain cysteines (e.g., about 85% or more, 90% or more, 95% or more of the binding affinity). An antibody as provided sometimes specifically binds to a cell surface molecule. In certain embodiments, the cell surface molecule is internalized in a cell. In some embodiments, the cell surface molecule is a cell surface receptor. In various embodiments, the cell surface receptor comprises a protein kinase domain. The cell surface receptor may be epidermal growth factor receptor (EGFR) protein tyrosine kinase. The cell surface receptor is sometimes a HER3 protein tyrosine kinase.

An antibody lacking native interchain cysteine amino acids may be conjugated to another molecule, in certain embodiments. In some embodiments, provided is an antibody conjugate in association with one or more heterologous molecules. In certain embodiments the antibody conjugate is about 80% or more of antibody products in a conjugation reaction product mixture (e.g., about 85% or more, 90% or more, 95% or more of the antibody products). In some embodiments the foregoing antibody comprises one or more cysteine replacements of non-cysteine surface amino acids in the CH1 domain, CH2 domain, or CH3 domain (to the extent such domains are present in the antibody), or combination thereof, of the antibody, where the one or more heterologous molecules are linked to the one or more cysteine replacements. In various embodiments the one or more heterologous molecules comprise a therapeutic agent. The therapeutic agent sometimes comprises a toxin. In some embodiments the one or more heterologous molecules comprise a diagnostic agent. In certain embodiments the diagnostic agent comprises an imaging agent, and sometimes, the diagnostic agent comprises a detectable label. In some embodiments, the one or more heterologous molecules are linked to the antibody via a linker.

In some embodiments, an antibody lacking interchain cysteines is part of an antibody homomultimer conjugate. Also provided, in certain embodiments, is the antibody comprising one or more cysteine replacements of non-cysteine surface amino acids in the CH1 domain, CH2 domain, or CH3 domain (to the extent such domains are present in the antibody), or combination thereof, of the antibody, where antibodies in the antibody homomultimer conjugate include a disulfide linkage between the one or more cysteine replacements.

Provided also, in some embodiments, is a nucleic acid comprising a nucleotide sequence that encodes an antibody herein. Also provided, in certain embodiments, is a cell comprising a nucleic acid that includes a nucleotide sequence encoding an antibody herein. In various embodiments, provided is an expression system that comprises a nucleic acid that includes a nucleotide sequence encoding an antibody herein. Also provided in certain embodiments is an organism that comprises a nucleic acid that includes a nucleotide sequence encoding an antibody herein. Provided also, in some embodiments, is an organism comprising an expression system that comprises a nucleic acid that includes a nucleotide sequence encoding an antibody herein.

Also provided, in certain embodiments, is a process that comprises expressing an antibody described herein in an expression system. In certain embodiments, such a method includes isolating the antibody, thereby producing an isolated antibody.

In some embodiments a process comprises conjugating an antibody described herein, often where the antibody is isolated, with a heterologous molecule, thereby preparing an antibody conjugate. In certain embodiments, provided is a process that comprises conjugating an antibody described herein, often where the antibody is in isolated form, with other isolated antibody, thereby preparing an antibody multimer.

Provided also, in some embodiments, is a method comprising contacting an antibody described herein with a biological sample, and detecting the presence, absence or amount of antibody specifically bound to a component in the biological sample. The method sometimes comprises linking the antibody to a solid support.

Also provided, in certain embodiments, is a method comprising administering an antibody described herein to cells and detecting the presence, absence or amount of antibody in a location of the cells. In some embodiments, provided is a method that comprises administering an antibody herein to a subject and detecting the presence, absence or amount of antibody in a tissue of the subject. A method sometimes comprises administering an antibody herein to cells, and detecting the presence, absence or amount of a biological effect associated with the administration of the antibody to the cells. In various embodiments, provided is a method that comprises administering an antibody herein to a subject, and detecting the presence, absence or amount of a biological effect in the subject associated with the administration of the antibody. In certain embodiments, the biological effect is cell proliferation inhibition. Provided also, in some embodiments, is a method that comprises administering an antibody herein to a subject, and monitoring the condition of the subject.

Certain embodiments are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments. Antibodies lacking native interchain cysteines are referred to as “FlexiMab” herein, and particularly as “mAb-Val” when all of the native interchain cysteines are substituted by the amino acid valine.

FIG. 1. Panel A is a schematic representation of a human antibody in its classic homodimeric format. In this format, the light and heavy chains are covalently linked by the formation of interchain disulphide bonds (one between the light and heavy chains, and two between the heavy chains at the hinge region). In addition to the interchain disulphide bonds, other non-covalent intermolecular interactions at the light and heavy chains and at the CH3 domains are necessary for the IgG homodimer formation and stabilization. Panel B shows a schematic representation of an antibody lacking interchain cysteines. The FlexiMab antibody is an antibody of the human IgG1 isotype with no interchain disulphide bonds. In mAb-Val, the cysteines at the light and heavy chain involved in the formation of the interchain disulphide bonds are mutated to Valine. mAb-Val does not form interchain disulphide bonds, but is able to assemble as a homodimer similar to a classic antibody, likely due to non-covalent intermolecular interactions at the light and heavy chains and at the CH3 domains.

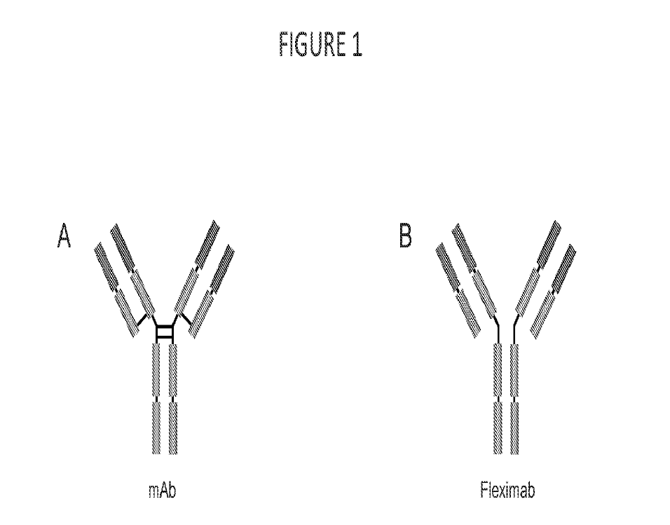

FIG. 2. Panel A is the amino acid sequence of a light chain kappa isotype of a classic anti-EGFR antibody format. The cysteine involved in the formation of the interchain disulphide bond with the heavy chain is represented in larger, bold and underlined text. Panel B is the amino acid sequence of a heavy chain gamma 1 isotype of a classic anti-EGFR antibody format. The cysteine involved in the formation of the interchain disulphide bond with the light chain, and the cysteines at the hinge region involved in the formation of the heavy interchain disulphide bonds are represented in larger, bold and underlined text. Panel C is the amino acid sequence of a light chain kappa isotype of a mAb-Val anti-EGFR antibody. The valine-to-cysteine substitution is represented in larger, bold and underlined text. Panel D is the amino acid sequence of a heavy chain gamma 1 isotype of a mAb-Val anti-EGFR antibody. The valine-to-cysteine substitutions are in larger, bold and underlined text.

FIG. 3. This table compares the expression and the monomeric content after protein A purification and overnight dialysis in standard antibody formulation buffer of an anti-EGFR antibody in its classic format (mAb) and a FlexiMab antibody in the mAb-Val format (mAb-Val). These data show that mAb and mAb-Val have similar expression levels when transiently expressed in mammalian cells and have similar monomeric content after traditional protein A purification and after overnight dialysis in classic antibody formulation buffer.

FIG. 4. This figure shows SDS-PAGE analysis in non-reducing and reducing condition for an anti-EGFR antibody in classic format (mAb) and a FlexiMab antibody in its mAb-Val format (mAb-Val). The mAb is a covalently linked antibody (in non-reducing condition run with the expected size of about 150 KDa); whereas the mAb-Val is a non-covalently linked homodimer and in non-reducing condition the heavy and light chain run as separate entities with the expected molecular weight (˜50 KDa for the heavy chain and ˜25 KDa for the light chain). In reducing conditions, both antibody formats run as independent chains with their predicted molecular weight sizes.

FIG. 5. This experiment compares the ELISA binding to coated EGFR of an anti-EGFR antibody in its classic format (mAb, filled circle) and a FlexiMab in its mAb-Val format (mAb-Val, open square). As shown in this figure, mAb and mAb-Val have similar binding signal to EGFR.

FIG. 6. This experiment compares the FACS binding to surface expressed EGFR on A431 cells, of an anti-EGFR antibody in its classic format (mAb) and a FlexiMab in its mAb-Val format (mAb-Val). As shown in this figure, mAb and mAb-Val exhibit similar binding signals to cell-surface expressed EGFR (they have similar mean-fluorescence-intensity values-MFIR-).

FIG. 7. This experiment compares ELISA binding to coated EGFR of an anti-EGFR antibody in its classic format (mAb, filled circle) and a FlexiMab in its mAb-Val format (mAb-Val, open square) after 5 days incubation at 37° C. in PBS. As shown in this figure, mAb and mAb-Val have similar binding signal to EGFR after 5 days incubation at 37° C. in PBS. This experiment suggests that mAb-Val is about as stable as mAb.

FIG. 8. This experiment compares ELISA binding to coated EGFR of an anti-EGFR antibody in its classic format (mAb, filled circle) and a FlexiMab in its mAb-Val format (mAb-Val, open square) after 5 days incubation at 37° C. in human serum. As shown in this figure, mAb and mAb-Val have similar binding signal to EGFR after 5 days incubation at 37° C. in human serum. This experiment suggest that mAb-Val is about as stable as mAb in an ex vivo experimental set up. The ELISA was carried out with 5% final human serum concentration.

FIG. 9. This table compares kinetic parameters (Kₒₙ, K_{off} and K_{d}) of anti-EGFR (mAb format) and anti-EGFR (a FlexiMab in mAb-Val format) for EGFR using a BIAcore assay. mAb and mAb-Val have similar binding affinity (in the pM range) for EGFR. EGFR was immobilized on the BIAcore surface chip.

FIG. 10. This experiment compares the inhibition of cancer cell survival (A431, BxPC3 and H358) of an anti-EGFR antibody in its classic format (mAb, open circle) and in a FlexiMab in its mAb-Val format (mAb-Val, open square). As a negative control a non-binding mAb is used (filled circle). As shown in this figure, mAb and mAb-Val exhibit similar potency in inhibiting the survival of human cancer cells.

FIG. 11. This experiment compares the rate and efficiency of internalization and therefore receptor degradation with subsequent cell killing of an anti-Her3 antibody in the classic mAb format (mAb) and in a FlexiMab in its mAb-Val format. In this experiment, cell killing is due to Saporin conjugated to an anti-human IgG1. SKBr3 cells expressing Her3 were used in this experiment. As shown in this experiment, anti-Her3 (mAb) and anti-Her3 (mAb-Val) have similar killing potency of SKBr3 cells. These results suggests that the mAb and mAb-Val have similar internalization kinetics. This assay was carried out by pre-incubating the two antibodies (mAb and mAb-Val) with an anti-human-IgG-Saporin-conjugated).

FIG. 12. This experiment compares the steady-state affinities (K_{D}) of anti-EGFR (mAb format) and anti-EGFR (FlexiMab in its mAb-Val format) for Fc-gamma-RIIIa (variant 158V and 158F) and for FcRn. The mAb-Val antibody exhibited no detectable binding to Fc-gamma-RIII. However, the mAb and mAb-Val exhibited similar binding affinity for FcRn. These results indicate that ADCC or CDC function is negatively impacted in the mAb-Val variant. The mAb and antibodies lacking interchain cysteines, however, are expected to have similar in vivo half-lives.

FIG. 13. This experiment compares the PK of anti-EGFR (mAb format, at 1 mg/kg filled circle and dashed line, and at 10 mg/kg filled square dashed line) and anti-EGFR (mAb-Val format, at 1 mg/kg filled triangle upside straight line, and at 0 mg/kg filled triangle downside straight line) in mice. mAb and mAb-Val have similar in vivo half-lifeves at both high dose (10 mg/kg) and low dose (1 mg/kg).

FIG. 14. This experiment compares the transition temperatures (raw data baseline subtracted) analyzed using differential scanning calorimetry for an anti-EGFR antibody in the mAb format (straight-line thermogram) and for FlexiMab in the mAb-Val format (dashed-line thermogram). This experiment shows the presence of two transition peaks for both antibody formats: one peak with a transition temperature of about 82° C. (similar for both antibody formats), and another peak with a transition temperature of 69° C. for the mAb and 65° C. for the mAb-Val. These results indicate that (as expected) the mAb-Val is slightly less stable than the mAb.

FIG. 15. This size-exclusion chromatography experiment shows that an anti-EGFR antibody in the FlexiMab mAb-Val format is mostly monomeric (˜98%) after protein A purification at 11 mg/ml in 25 mM Histidine-HCl pH 6.

FIG. 16. This experiment compares in vivo efficacy (left panel) and body weight (right panel) for an anti-EGFR antibody in classic format (mAb, filled circle) and in FlexiMab in its mAb-Val format (open square). Negative controls include untreated animals (filled rhomb) and animals treated with a mAb isotype control (filled square). The animals were dosed 2 times per week for the entire duration of the experiment. The efficacy study (left panel) shows that the mAb and mAb-Val exhibit similar in vivo efficacy. In addition (right panel), mAb and mAb-Val have similar toxicity (i.e., there is not a significant difference in total body weight lost for animal treated with mAb and mAb-Val), which compare with control vehicles. The tumor model used in this study is A431 (human epidermoid carcinomas).

FIG. 17. This figure shows a schematic ribbon representation of mAb-Val (right panel) with the Valine-to-Cysteine substitutions indicated with filled dots (right panel). The Ser-131 to Thr-139 CH1 loop containing the Cysteine-substitutions is shown in black (right panel). The left panel is a zoom view of the Ser-131 to Thr-139 CH1 loop that shows the structural location of the Ser-131, Ser-132, Ser-134, Thr-135, Ser-136 and Thr-139 (EU numbering) positions. See also, Table 2.

FIG. 18. This table shows transient expression in mammalian cells after 7 days post-transfection and the monomeric content after protein A purification in PBS for selected mAb-Val Cysteine mutants. These mutants have been designed to be used for site-specific conjugation. The mutants are single (Ser131Cys, Ser132Cys, Ser134Cys, Thr135Cys, Ser136Cys andThr139Cys), double (Ser131Cys-Thr139Cys) and triple mutants (Ser131Cys-Thr135Cys-Thr139Cys). This combination of mutations can allow the site-specific conjugation of two (single mutant), four (double mutants) and six (triple mutants) druglike entities per antibody. As shown in the table, all mutants have expression comparable to mAb (compare with values reported in FIG. 3) and are ˜96% monomeric after protein A purification and overnight dialysis in PBS. Antibody preparation for site-specific conjugation, site-specific conjugations and analyses were carried out as described in International Application Publication No. WO 2009/092011 entitled “Cysteine engineered antibodies for site-specific conjugation.”

FIG. 19. This figure shows show the peptide mapping results of anti-EGFR mAb-Val Cysteine-engineered constructs that have been conjugated using Maleimide-PEG2-Biotin as described in U.S. Patent Application Publication No. 2009092011. The figure shows the mAb-Val technology can afford a nearly homogeneous site-specific drug conjugation for two, four and six drug-like entities per antibody.

FIG. 20. This figure shows the amino acid sequence at the hinge region for a mAb and a FlexiMab in its mAb-Val format (SEQ ID NOS 20 and 21, respectively). The Valine and Cysteine position are underlined. In mAb-Val the Threonine shown with the arrow is O-glycosylated, as determined experimentally by intact mass and peptide mapping on an anti-EGFR mAb-Val construct. This hinge O-glycosylation could make the hinge region in mAb-Val less susceptible to in vivo protease cleavage.

FIG. 21. This figure shows constant region amino acid sequences for IgG1, IgG2, IgG3 and IgG4 antibody isotypes and interchain cysteine amino acids that are modified in FlexiMab antibodies described herein. The figure also designates CH1, CH2, CH3 and hinge regions in the heavy chain constant region. Amino acid sequences for IgG1, IgG2, IgG3 and IgG4 heavy chain constant regions, and kappa and lambda light chain constant regions, are designated by SEQ ID NOs: 5, 6, 7, 8, 9 and 10, respectively, and are reproduced herein.

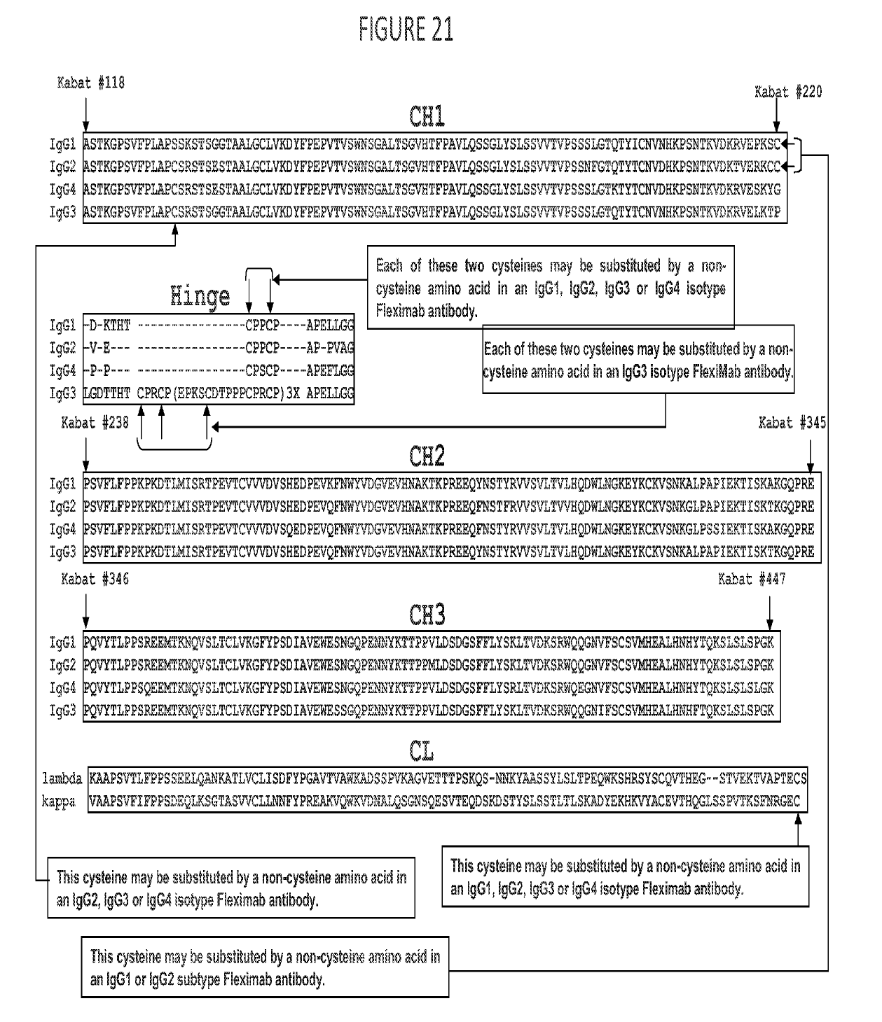

### DETAILED DESCRIPTION

Provided herein, as more fully discussed below, are engineered antibodies in which all of the naturally occurring interchain cysteine amino acids are substituted with a non-thiol amino acid. Such antibodies are referred to as “antibodies lacking interchain cysteines.” Monoclonal antibodies in which native interchain cysteines are substituted are referred to as “FlexiMab” and antibodies in which native interchain cysteines are substituted by the amino acid valine are referred to herein as “mAb-Val.” Also provided herein, as more fully discussed below, are antibodies lacking interchain cysteines in which selected intra-chain amino acids are substituted with cysteine.

Antibodies are large, complex and structurally diverse biomolecules, often with many reactive functional groups. Their reactivities with linker reagents and druglinker intermediates are dependent on factors such as pH, concentration, salt concentration, and co-solvents. Furthermore, the multistep conjugation process may be non-reproducible due to difficulties in controlling the reaction conditions and characterizing reactants and intermediates.

Methods are known for attaching a heterologous molecule to an antibody. The molecule sometimes is a detectable label and sometimes is a drug. Conventional means of attaching, i.e. linking through covalent bonds, a heterologous moiety to an antibody generally leads to a heterogeneous mixture of molecules where the heterologous moieties are attached at a number of sites on the antibody. For example, cytotoxic drugs have typically been conjugated to antibodies through the often-numerous lysine or cysteine residues of an antibody, generating a heterogeneous antibody-drug conjugate mixture.

Depending on reaction conditions, the mixture resulting from a conjugation reaction typically contains a distribution of antibodies with from 0 to about 8, or more, attached heterologous moieties. In addition, within each subgroup of conjugates with a particular integer ratio of drug moieties to antibody, is a potentially heterogeneous mixture where the heterologous moiety is attached at various sites on the antibody. Antibodies described herein, which lack all naturally occurring interchain cysteine amino acids, reduce the number of sites to which the heterologous moiety can bind, thereby reducing the heterogeneity of the product mixture. Reducing heterogeneity among reaction products can advantageously result in a more homogeneous and useable product for diagnostic and therapeutic applications. More homogeneity can lead to more certainty in the structure of a therapeutic or diagnostic agent.

Cysteine thiols are reactive at neutral pH, unlike most amines which are protonated and less nucleophilic near pH 7. Since free thiol (R—SH, sulfhydryl) groups are relatively reactive, proteins with cysteine residues often exist in their oxidized form as disulfide-linked oligomers or have internally bridged disulfide groups. The amount of free thiol in a protein may be estimated by the standard Ellman's assay. IgM is an example of a disulfide-linked pentamer, while IgG is an example of a protein with internal disulfide bridges bonding the subunits together. In proteins such as this, reduction of the disulfide bonds with a reagent such as dithiothreitol (DTT) or selenol is required to generate the reactive free thiol. This approach may result in loss of antibody tertiary structure and antigen binding specificity. In certain embodiments, an antibody lacking interchain cysteine amino acids can include one or more engineered cysteines, the latter of which can be conjugated to a heterologous moiety.

### Terminology

Methods provided herein often are not limited to specific compositions or process steps, as such may vary. Also, as used herein, the singular form “a”, “an” and “the” include plural referents unless the context clearly dictates otherwise.

Amino acids often are referred to herein by commonly known three letter symbols or by the one-letter symbols recommended by the IUPAC-IUB Biochemical Nomenclature Commission. Nucleotides, likewise, often are referred to by commonly accepted single-letter codes.

The numbering of amino acids in the variable domain, complementarity determining region (CDRs) and framework regions (FR), of an antibody follow, unless otherwise indicated, the Kabat definition as set forth in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insertion (residue 52a according to Kabat) after residue 52 of H2 and inserted residues (e.g. residues 82a, 82b, and 82c, etc according to Kabat) after heavy chain FR residue 82. The Kabat numbering of residues may be determined for a given antibody by alignment at regions of homology of the sequence of the antibody with a “standard” Kabat numbered sequence. Maximal alignment of framework residues frequently requires the insertion of “spacer” residues in the numbering system, to be used for the Fv region. In addition, the identity of certain individual residues at any given Kabat site number may vary from antibody chain to antibody chain due to interspecies or allelic divergence.

### Antibodies

Antibodies are immunological proteins that bind a specific antigen. In most mammals, including humans and mice, antibodies are constructed from paired heavy and light polypeptide chains. Each chain is made up of two distinct regions, referred to as the variable (Fv) and constant (Fc) regions. The light and heavy chain Fv regions contain the antigen binding determinants of the molecule and are responsible for binding the target antigen. The Fc regions define the class (or isotype) of antibody (IgG for example) and are responsible for binding a number of natural proteins to elicit important biochemical events.

Each light chain is linked to a heavy chain by one covalent disulfide bond, while the number of disulfide linkages varies between the heavy chains of different immunoglobulin isotypes. Each heavy and light chain also has regularly spaced interchain disulfide bridges. The two light chain-heavy chain dimers are linked via disulfide bridges between the heavy chains, forming a Y-shaped molecule. The region in which the arms of the Y meet the stem is called the hinge region, and exhibits some flexibility.

Each chain includes constant regions that are representative of the antibody class and variable regions specific to each antibody. The constant region determines the mechanism used to destroy antigen. Antibodies are divided into five major classes, IgM, IgG, IgA, IgD, and IgE, based on their constant region structure and immune function. The variable and constant regions of both the light and the heavy chains are structurally folded into functional units called domains. Each light chain consists of one variable domain (VL) at one end and one constant domain (CL) at its other end. Each heavy chain has at one end a variable domain (VH) followed by three or four constant domains (CH1, CH2, CH3, CH4).

The arms of the Y contain the site that bind antigen and is called the Fab (fragment, antigen binding) region. It is composed of one constant and one variable domain from each heavy and light chain of the antibody. The constant domain of the light chain is aligned with the first constant domain of the heavy chain, and the light chain variable domain is aligned with the variable domain of the heavy chain. Light chains are classified as lambda chains or kappa chains based on the amino acid sequence of the light chain constant region. The variable domain of a kappa light chain may also be denoted herein as VK.

The Fc region of an antibody interacts with a number of ligands including Fc receptors and other ligands, imparting an array of important functional capabilities referred to as effector functions. An important family of Fc receptors for the IgG class are the Fc gamma receptors (FcγRs). These receptors mediate communication between antibodies and the cellular arm of the immune system In humans this protein family includes FcγRI (CID64), including isoforms FcγRIA, FcγRIB, and FcγRIC; FcγRII (CD32), including isoforms FcγRIIA, FcγRIIB, and FcγRIIC; and FcγRIII (CD16), including isoforms FcγRIIIA and FcγRIIB. These receptors typically have an extracellular domain that mediates binding to Fc, a membrane spanning region, and an intracellular domain that may mediate some signaling event within the cell. These different FcγR subtypes are expressed on different cell types. For example, in humans, FcγRIIIB is found only on neutrophils, whereas FcγRIIIA is found on macrophages, monocytes, natural killer (NK) cells, and a subpopulation of T-cells.

Formation of the Fc/FcγR complex recruits effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. The cell mediated reaction where nonspecific cytotoxic cells that express FcγRs recognize bound antibody on a target cell and subsequently cause lysis of the target cell is referred to as antibody dependent cell-mediated cytotoxicity (ADCC). The primary cells for mediating ADCC, NK cells, express only FcγRIIIA, whereas monocytes express FcγRI, FcγRII and FcγRIII.

Another important Fc ligand is the complement protein C1q. Fc binding to C1q mediates a process called complement dependent cytotoxicity (CDC). C1q is capable of binding six antibodies, although binding to two IgGs is sufficient to activate the complement cascade. C1q forms a complex with the C1r and C1s serine proteases to form the C1 complex of the complement pathway.

Several key features of antibodies including but not limited to, specificity for target, ability to mediate immune effector mechanisms, and long half-life in serum, make antibodies and related immunoglobulin molecules powerful therapeutics. There are a number of possible mechanisms by which antibodies destroy tumor cells, including anti-proliferation via blockage of needed growth pathways, intracellular signaling leading to apoptosis, enhanced down regulation and/or turnover of receptors, ADCC, CDC, and promotion of an adaptive immune response.

As used herein, the terms “antibody” and “antibodies”, also known as immunoglobulins, encompass monoclonal antibodies (including full-length monoclonal antibodies), polyclonal antibodies, multispecific antibodies formed from at least two different epitope binding fragments (e.g., bispecific antibodies), human antibodies, humanized antibodies, camelised antibodies, chimeric antibodies, single-chain Fvs (scFv), single-chain antibodies, single domain antibodies, domain antibodies. Fragments of antibodies include Fab fragments, F(ab′)2 fragments, antibody fragments that exhibit the desired biological activity (e.g. the antigen binding portion), disulfide-linked Fvs (dsFv), and anti-idiotypic (anti-Id) antibodies (including, e.g., anti-Id antibodies to antibodies herein provided), intrabodies, and epitope-binding fragments of any of the above. In particular, antibodies include immunoglobulin molecules and immunologically active fragments of immunoglobulin molecules, i.e., molecules that contain at least one antigen-binding site. Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., G1m(f, z, a or x), G2m(n), G3m(g, b, or c), Am, Em, and Km(1, 2 or 3)). Antibodies may be derived from any mammal, including, but not limited to, humans, monkeys, pigs, horses, rabbits, dogs, cats, mice, and the like, or other animals such as birds (e.g. chickens). Where a particular antibody or antibody fragment lacks the residues that are substituted in a FexiMab (e.g., a scFV), that antibody or antibody fragment may be fused to an Fc or other portion of an antibody in the FexiMab format.

Antibodies provided herein include full length or intact antibody, antibody fragments, native sequence antibody or amino acid variants, human, humanized, post-translationally modified, chimeric or fusion antibodies, immunoconjugates, and functional fragments thereof. The antibodies can be modified in the Fc region, and certain modifications can provide desired effector functions or serum half-life. As discussed in more detail in the sections below, with the appropriate Fc regions, a naked antibody bound on the cell surface can induce cytotoxicity, e.g., via antibody-dependent cellular cytotoxicity (ADCC) or by recruiting complement in complement dependent cytotoxicity (CDC), or by recruiting nonspecific cytotoxic cells that express one or more effector ligands that recognize bound antibody on a target cell and subsequently cause phagocytosis of the target cell in antibody dependent cell-mediated phagocytosis (ADCP), or some other mechanism. Where it is desirable to eliminate or reduce effector function, so as to minimize side effects or therapeutic complications, certain other Fc regions may be used. The Fc region of antibodies can be modified to increase the binding affinity for FcRn and thus increase serum half-life. Alternatively, the Fc region can be conjugated to PEG or albumin to increase the serum half-life, or some other conjugation that results in a desired effect.

In certain embodiments, an antibody herein is isolated and/or purified and/or pyrogen free antibodies. The term “purified” as used herein, refers to a molecule of interest that has been identified and separated and/or recovered from a component of its natural environment. Thus, in some embodiments, an antibody provided is a purified antibody where it has been separated from one or more components of its natural environment. The term “isolated antibody” as used herein refers to an antibody which is substantially free of other antibody molecules having different structure or antigenic specificities. A bi- or multi-specific antibody molecule is an isolated antibody when substantially free of other antibody molecules. Thus, in some embodiments, antibodies provided are isolated antibodies which have been separated from antibodies with a different specificity. An isolated antibody may be a monoclonal antibody. An isolated antibody that specifically binds to an epitope, isoform or variant of a target may, however, have cross-reactivity to other related antigens, e.g., from other species (e.g., species homologs). An isolated antibody as provided may be substantially free of one or more other cellular materials. In some embodiments, a combination of “isolated” monoclonal antibodies is provided, and pertains to antibodies having different specificities and combined in a defined composition. Methods of production and purification/isolation of an antibody are described elsewhere herein.

Isolated antibodies presented comprise antibody amino acid sequences disclosed herein, which can be encoded by any suitable polynucleotide. Isolated antibodies sometimes are provided in formulated form. In some embodiments, an antibody binds to a protein and, thereby partially or substantially alters at least one biological activity of the protein, for example, cellular proliferation activity.

Humanized Antibodies

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains in which all or substantially all of the CDR regions correspond to those of a non-human immunoglobulin (i.e., donor antibody) and all or substantially all of the framework regions are those of a human immunoglobulin consensus sequence. A humanized antibody may also comprise at least a portion of an immunoglobulin constant region (Fc), typically that of a human immunoglobulin. The antibody may contain both the light chain as well as at least the variable domain of a heavy chain. The antibody also may include the CH1, hinge, CH2, CH3, and CH4 regions of the heavy chain.

The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG1. Where such cytotoxic activity is not desirable, the constant domain may be of the IgG2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, may not be extensive. At least 75% of the humanized antibody residues may correspond to those of the parental framework region (FR) and CDR sequences, with the correspondence sometimes being 90% or greater or 95% or greater, for example.

Humanization can be essentially performed following methods known in the art, by substituting hypervariable region sequences for the corresponding sequences of a human antibody. Specifically, humanized antibodies may be prepared by methods known in the art including CDR grafting approaches, veneering or resurfacing, chain shuffling strategies, molecular modeling strategies, and the like. These general approaches may be combined with standard mutagenesis and recombinant synthesis techniques to produce antibodies herein with desired properties.

CDR grafting is performed by replacing one or more CDRs of an acceptor antibody (e.g., a human antibody) with one or more CDRs of a donor antibody (e.g., a non-human antibody). Acceptor antibodies may be selected based on similarity of framework residues between a candidate acceptor antibody and a donor antibody and may be further modified to introduce similar residues. Following CDR grafting, additional changes may be made in the donor and/or acceptor sequences to optimize antibody binding and functionality.

Grafting of abbreviated CDR regions is a related approach. Abbreviated CDR regions include the specificity-determining residues and adjacent amino acids, including those at positions 27d-34, 50-55 and 89-96 in the light chain, and at positions 31-35b, 50-58, and 95-101 in the heavy chain. Grafting of specificity-determining residues (SDRs) is premised on the understanding that the binding specificity and affinity of an antibody combining site is determined by the most highly variable residues within each of the CDR regions. Analysis of the three-dimensional structures of antibody-antigen complexes, combined with analysis of the available amino acid sequence data was used to model sequence variability based on structural dissimilarity of amino acid residues that occur at each position within the CDR. Minimally immunogenic polypeptide sequences consisting of contact residues, which are referred to as SDRs, are identified and grafted onto human framework regions.

Veneering or resurfacing is based on the concept of reducing potentially immunogenic amino acid sequences in a rodent or other non-human antibody by resurfacing the solvent accessible exterior of the antibody with human amino acid sequences. Thus, veneered antibodies appear less foreign to human cells. A non-human antibody is veneered by (1) identifying exposed exterior framework region residues in the non-human antibody, which are different from those at the same positions in framework regions of a human antibody, and (2) replacing the identified residues with amino acids that typically occupy these same positions in human antibodies.

By definition, humanized antibodies are chimeric antibodies. Chimeric antibodies are antibodies in which a portion of the heavy and/or light chain is identical with or homologous to corresponding sequences in antibodies derived from a particular species or belonging to a particular antibody class or subclass, while another portion of the chain(s) is identical with or homologous to corresponding sequences in antibodies derived from another species or belonging to another antibody class or subclass, as well as fragments of such antibodies, so long as they exhibit the desired biological activity. Chimeric antibodies of interest herein include “primatized” antibodies comprising variable domain antigen-binding sequences derived from a nonhuman primate (e.g., Old World Monkey, such as baboon, rhesus or cynomolgus monkey) and human constant region sequences.

Provided herein are engineered antibodies that can be utilized to generate anti-idiotype antibodies using techniques known in the art. Also provided herein are methods employing the use of polynucleotides comprising a nucleotide sequence encoding an antibody herein or a fragment thereof. Additionally, various methods are known in the art for obtaining physiologically active molecules whose half-lives are modified either by introducing an FcRn-binding polypeptide into the molecules or by fusing the molecules with antibodies whose FcRn-binding affinities are preserved but affinities for other Fc receptors have been greatly reduced or fusing with FcRn binding domains of antibodies. Specific techniques and methods of increasing half-life of physiologically active molecules are additionally known in the art. Specifically, it is contemplated that the antibodies herein comprise an Fc region comprising amino acid residue mutations (as numbered by the EU index in Kabat): M252Y/S254T/T256E or H433K/N434F/Y436H.

Human Antibodies

For some uses, including in vivo use of antibodies in humans and in vitro detection assays, it may be appropriate to use human or chimeric antibodies. Completely human antibodies may be desirable for therapeutic treatment of human subjects. Human antibodies can be made by a variety of methods known in the art including phage display methods described below using antibody libraries derived from human immunoglobulin sequences.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of an antibody herein.

Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. In addition, companies such as Medarex (Princeton, N.J.) provide human antibodies directed against a selected antigen.

Chimeric antibodies comprising one or more CDRs from a non-human species and framework regions from a human immunoglobulin molecule can be produced using a variety of techniques known in the art including, for example, CDR-grafting veneering or resurfacing.

Framework residues in the framework regions may be substituted with the corresponding residue from the CDR donor antibody to alter, and potentially improve, antigen binding. These framework substitutions are identified by methods known in the art, e.g., by modeling of the interactions of the CDR and framework residues to identify framework residues important for antigen binding and sequence comparison to identify unusual framework residues at particular positions. Also known in the art is a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and usually a second constant region (sometimes a gamma constant region) are formed into a construct for insertion into an animal.

The generation of human antibodies from mice in which, through microcell fusion, large pieces of chromosomes, or entire chromosomes, have been introduced, is also known in the art. For example, cross-breeding of Kirin's Tc mice with Medarex's minilocus (Humab) mice has generated mice possessing the human IgH transchromosome of the Kirin mice and the kappa chain transgene of the Genpharm mice.

Human antibodies can also be derived by in vitro methods. Suitable examples include but are not limited to phage display (Medlmmune (formerly CAT), Morphosys, Dyax, Biosite/Medarex, Xoma, Symphogen, Alexion (formerly Proliferon), Affimed) ribosome display (Medlmmune (formerly CAT)), yeast display, and the like. The phage display technology can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats as known in the art. A diverse array of anti-oxazolone antibodies has been isolated from a small random combinatorial library of V genes derived from the spleens of immunized mice. A repertoire of V genes from unimmunized human donors can be constructed and antibodies to a diverse array of antigens (including self-antigens) can be isolated essentially following the techniques known in the art. Human antibodies may also be generated by in vitro activated B cells.

Multivalent Antibodies

Antibodies with more than two valencies are contemplated. For example, trispecific antibodies can be prepared. Thus, the antibodies herein presented can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. In an embodiment a dimerization domain comprises (or consists of) an Fc region or a hinge region. In this scenario, the antibody may comprise an Fc region and three or more antigen binding sites amino-terminal to the Fc region. In certain embodiments the multivalent antibody herein comprises (or consists of) three to about eight antigen binding sites. The multivalent antibody comprises at least one polypeptide chain where the polypeptide chain(s) comprise two or more variable domains. For instance, the polypeptide chain(s) may comprise VD1-(X1)n-VD2-(X2)n-Fc, where VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

Bi-Specific Antibodies

Bispecific antibodies are antibodies that have binding specificities for at least two different epitopes. Exemplary bispecific antibodies may bind to two different epitopes of a target protein, or may bind two polypeptides. Other such antibodies may combine a site with a binding site for another protein. An bi-specific antibody arm may sometimes be combined with an arm which binds to a triggering molecule on a leukocyte such as a T-cell receptor molecule (e.g. CD3), or Fc receptors for IgG (FcγR), such as FcγRI (CD64), FcγRII (CD32) and FcγRIII (CD16), so as to focus and localize cellular defense mechanisms to the target protein-expressing cell. Bispecific antibodies may also be used to localize cytotoxic agents to cells which express the antigen. Such antibodies may possess a target-binding arm and an arm which binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab′)2 bispecific antibodies). Methods for making bispecific antibodies are known in the art.

Traditional production of full length bispecific antibodies is based on the co-expression of two immunoglobulin heavy chain-light chain pairs, where the two chains have different specificities Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low.

In another approach, antibody variable domains with the desired binding specificities (antibody-antigen combining sites) are fused to immunoglobulin constant domain sequences. A fusion sometimes is with an Ig heavy chain constant domain, comprising at least part of the hinge, CH2, and CH3 regions. The first heavy-chain constant region (CH1) containing the site necessary for light chain bonding may be present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

In some embodiments of this approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure may facilitate the separation of the desired bispecific compound from unwanted immunoglobulin chain combinations, as the presence of an immunoglobulin light chain in only one half of the bispecific molecule provides for a facile way of separation.

The interface between a pair of antibody molecules may be engineered to maximize the percentage of heterodimers which are recovered from recombinant cell culture. An appropriate interface comprises at least a part of the CH3 domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods. Suitable cross-linking agents are known in the art.

Bispecific antibodies may be generated from antibody fragments. For example, bispecific antibodies can be prepared using chemical linkage. In one procedure intact antibodies are proteolytically cleaved to generate F(ab′)2 fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′-SH fragments can be directly recovered from E. coli, which can be chemically coupled to form bispecific antibodies. A fully humanized bispecific antibody F(ab′)2 molecule may be created by secreting each Fab′ fragment separately from E. coli and subjecting to directed chemical coupling in vitro to form the bispecific antibody. The bispecific antibody thus formed is able to bind to cells over expressing the ErbB2 receptor and normal human T cells, as well as trigger the lytic activity of human cytotoxic lymphocytes against human breast tumor targets.

Bispecific antibodies may also be produced using leucine zippers. The leucine zipper peptides from the Fos and Jun proteins are linked to the Fab′ portions of two different antibodies by gene fusion. The antibody homodimers are reduced at the hinge region to form monomers and then re-oxidized to form the antibody heterodimers. This method can also be utilized for the production of antibody homodimers. The “diabody” technology described has provided an additional mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers is also known in the art.

### Antibody Receptors

Cells may display specialized antigens called antibody receptors to which an antibody specifically binds as discussed above. Some receptors are configured to be internalized into the cell. An internalizing receptor may carry a bound antibody and conjugated molecules with it. This property makes antibodies potentially valuable for therapy, since pathogenic cells, including cancer cells, may display unique antigens. Cellular receptors may display functional regions, including protein kinase.

The kinase family is one of the largest target families in the human genome. It is estimated that the human genome includes more than 500 members of the major classes of protein serine/threonine, tyrosine, and dual specificity kinases. Protein phosphorylation is a significant signal transduction mechanisms by which intercellular signals regulate crucial intracellular processes such as ion transport, cellular proliferation, and hormone responses. Growth factor receptors EGFR and HER3 and protein tyrosine kinases. The protein kinase family's key function in signal transduction for all organisms makes it an attractive target class for therapeutic interventions in a number of disease states such as cancer, diabetes, inflammation, and arthritis.

Epidermal growth factor (EGF)-enhanced protein kinase activity in plasma membrane preparations of A-431 human epidermoid carcinoma cells has been shown to involve the phosphorylation of tyrosine residues. The epidermal growth factor receptor (EGFR, ErbB-1, HER1, HER3 in humans) is the cell-surface receptor for members of the EGF-family of extracellular protein ligands. The epidermal growth factor receptor is a member of the ErbB family of receptors, a subfamily of four closely related receptor tyrosine kinases: EGFR (ErbB-1), HER2/c-neu (ErbB-2), Her 3 (ErbB-3) and Her 4 (ErbB-4). All four members of the human epidermal growth factor (EGF) receptor (HER) family are implicated in human cancers.

Provided herein, in some embodiments, are antibodies lacking interchain cysteines that bind to EGFR and HER3. Also provided, in certain embodiments, are antibody lacking interchain cysteines conjugates that bind to EGFR and HER3. The antibody conjugates are sometimes internalized.

### Antibody Function

Antibodies can effect several functions, such as antigen binding and inducing an immune response, for example.

Antigen Binding

The term “antigen” as used herein refers to a molecule that causes an immune response when introduced into an organism and that is capable of binding with specific antibodies. Antibody-antigen binding is mediated by the sum of many weak interactions between the antigen and antibody including, for example, hydrogen bonds, van der Waals forces, and ionic and/or hydrophobic interactions.

An antigen binds to the complementarity regions on an antibody. The corresponding region(s) of the antigen is referred to as an antigenic determinant. Most antigens have multiple determinants; if 2 or more are identical, the antigen is multivalent.

The affinity of an antibody reflects the fit between the antigenic determinant and the antibody binding site and is independent of the number of binding sites. The avidity of the binding reflects the overall stability of the antibody-antigen complex. Avidity is defined as the total binding strength of all binding sites. Thus, both affinity of the antibody for its antigen and the valencies of both the antibody and the antigen influence avidity. Engagement of both, rather than only one, multivalent binding sites may strengthen binding by a factor of as much as 10,000 in a typical IgG molecule.

The multivalent nature of many antibodies and antigens may give rise to secondary reactions such as precipitation, cell clumping, and complement fixation in an organism. Such reactions can be useful in techniques such as western blotting, ELISA, immunoprecipitation, and the like.

### Immune Function

Antibodies bind and inactivate pathogens, can stimulate removal of pathogens by macrophages and other cells by coating the pathogen, and trigger destruction of pathogens by stimulating other immune responses such as the complement pathway. Antibodies activate the complement pathway by binding to surface antigens on, for example, a bacterium or cancer cell. The Fc region of the antibody then interacts with the complement cascade. The binding of the antibody and of complement cascade molecules attracts phagocytes and marks the microbe or cell for ingestion. Complement system components may form a membrane attack complex to assist antibodies to kill the bacterium or cell directly.

Antibody binding may cause pathogens to agglutinate. Antibody coated pathogens stimulate effector functions in cells that recognize the antibody Fc region. The effector function ultimately results in destruction of the invading microbe or pathogenic cell, e.g. phagocytes will phagocytose, mast cells and neutrophils will degranulate, natural killer cells will release cytokines and cytotoxic molecules.

Transformed tumor cells express abnormal antigens from several sources, including oncogenic viruses, abnormally high levels of the organism's own proteins, and cancer inducing oncogenes. Tumor antigens are presented on major histo-compatiblity (MHC) class I molecules in a manner similar to viral antigens. Antigens activate killer T-Cells and also generate antibodies that trigger the complement system.

An antibody herein may bind to a tumor or other pathogenic cell antigen and trigger cell destruction through an antibody function. In certain embodiments, an antibody herein may be conjugated to a therapeutic molecule, including a diagnostic molecule or toxin, and carry the conjugated molecule to selected site by means of antibody-antigen affinity.

### Epitopes

The term “epitope” as used herein refers to a protein determinant capable of binding to an antibody. Epitopes generally include chemically active surface groupings of molecules such as amino acids and/or sugar side chains and generally have specific three dimensional structural characteristics, as well as specific chemical characteristics (e.g., charge, polarity, basic, acidic, hydrophobicity and the like). Conformational and non-conformational epitopes are distinguished in that the binding to the former but not the latter is lost in the presence of denaturing solvents.

In certain embodiments, an epitope is comprised of at least one extracellular, soluble, hydrophilic, external or cytoplasmic portion of a target protein. A specified epitope can comprise any combination of at least one amino acid sequence from of at least 3 amino acid residues to the entire specified portion of contiguous amino acids of the target protein. In some embodiments, the epitope is at least 4 amino acid residues, at least 5 amino acid residues, at least 6 amino acid residues, at least 7 amino acid residues, at least 8 amino acid residues or at least 9 amino acid residues to the entire specified portion of contiguous amino acids of the target protein.

An antibody herein may immunospecifically bind to one or more epitopes specific to the target protein, peptide, subunit, fragment, portion or any combination thereof and generally does not specifically bind to other polypeptides. An epitope may comprise at least one antibody binding region that comprises at least one portion of the target protein.

### Antibody Fragments

In certain embodiments, the present antibodies are antibody fragments or antibodies comprising these fragments. The antibody fragment comprises a portion of the full length antibody, which generally is the antigen binding or variable region thereof. Examples of antibody fragments include Fab, Fab′, F(ab′)2, Fd and Fv fragments. Diabodies; linear antibodies; single-chain antibody molecules; and multispecific antibodies are antibodies formed from these antibody fragments.

Traditionally, these fragments were derived via proteolytic digestion of intact antibodies using techniques known in the art. However, these fragments can now be produced directly by recombinant host cells. Fab, Fv and scFv antibody fragments can all be expressed in and secreted from E. coli, thus allowing the facile production of large amounts of these fragments. In an embodiment, the antibody fragments can be isolated from the antibody phage libraries discussed elsewhere herein. Fab′-SH fragments can also be directly recovered from E. coli and chemically coupled to form F(ab′)2 fragments. F(ab′)2 fragments can also be isolated directly from recombinant host cell culture. Other techniques for the production of antibody fragments are known in the art. In various embodiments, the antibody of choice is a single-chain Fv fragment (scFv). In certain embodiments, the antibody is not a Fab fragment. Fv and scFv are the only species with intact combining sites that are devoid of constant regions; thus, they are suitable for reduced nonspecific binding during in vivo use. scFv fusion proteins may be constructed to yield fusion of an effector protein at either the amino or the carboxy terminus of an scFv.

In certain embodiments, the present antibodies are domain antibodies, e.g., antibodies containing the small functional binding units of antibodies, corresponding to the variable regions of the heavy (VH) or light (VL) chains of human antibodies. Examples of domain antibodies include, but are not limited to, those available from Domantis that are specific to therapeutic targets. Commercially available libraries of domain antibodies can be used to identify antigen domain antibodies. In certain embodiments, antibodies herein comprise a functional binding unit and an Fc gamma receptor functional binding unit.

In certain embodiments, the present antibodies are vaccibodies. Vaccibodies are dimeric polypeptides. Each monomer of a vaccibody consists of a scFv with specificity for a surface molecule on APC connected through a hinge region and a Cy3 domain to a second scFv. In some embodiments, vaccibodies containing as one of the scFv's an antibody lacking interchain cysteines fragment may be used to juxtapose those cells to be destroyed and an effector cell that mediates ADCC.

In certain embodiments herein, the present antibodies are linear antibodies. Linear antibodies comprise a pair of tandem Fd segments (VH-CH1-VH-CH1) which form a pair of antigen-binding regions. Linear antibodies may be bispecific or monospecific.

### Antibody Synthesis

An antibody lacking interchain cysteines may be produced by any method known in the art for the synthesis of antibodies, in particular, by chemical synthesis or by recombinant expression techniques.

Any antigen can be used to synthesize an antibody. Examples of antigens, or “targets,” are described herein. Cells expressing the desired antigen at their cell surface or membranes prepared from such cells can also be used to generate antibodies. Antibodies herein can be produced recombinantly in an isolated from in bacterial or eukaryotic cells using standard recombinant DNA methodology. Antigen can be expressed as a tagged (e.g., epitope tag) or other fusion protein to facilitate isolation as well as identification in various assays. Antibodies or binding proteins that bind to various tags and fusion sequences are available as elaborated below. Other forms of antigens useful for generating antibodies herein will be apparent to those skilled in the art.

Various tag polypeptides and their respective antibodies are known in the art. Examples include: poly-histidine (poly-his) or poly-histidine-glycine (poly-his-gly) tags; the flu HA tag polypeptide and its antibody 12CA5; the c-myc tag and the 8F9, 3C7, 6E10, G4, B7 and 9E10 antibodies thereto; and the Herpes Simplex virus glycoprotein D (gD) tag and its antibody. The FLAG-peptide is recognized by an anti-FLAG M2 monoclonal antibody. Purification of a protein containing the FLAG peptide can be performed by immunoaffinity chromatography using an affinity matrix comprising the anti-FLAG M2 monoclonal antibody covalently attached to agarose. Other tag polypeptides include the KT3 epitope peptide, an α-tubulin epitope peptide, and the T7 gene 10 protein peptide tag.

Polyclonal antibodies, which bind to more than one epitope on an antigen, are raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent (reactive group), e.g., activated ester (conjugation through cysteine or lysine residues), glutaraldehyde, succinic anhydride, SOCl2, or R1N═C═NR, where R and R1 are different alkyl groups. Conjugates also can be made in recombinant cell culture as fusion proteins.

Polyclonal antibodies to an antigen-of-interest can be produced by various procedures known in the art. For example, an antigenic polypeptide or immunogenic fragment thereof can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also known in the art.

Animals may be immunized against the antigen, immunogenic conjugates, or derivatives by combining an appropriate concentration of antigen or conjugate with adjuvant and injecting the solution at multiple sites. One month later, the animals are boosted with ⅕ to 1/10 the original amount of antigen or conjugate in adjuvant by subcutaneous injection at multiple sites. Seven to 14 days later, the animals are bled and the serum is assayed for antibody titer. Animals are boosted until the titer plateaus. In addition, aggregating agents such as alum are suitably used to enhance the immune response.

Monoclonal antibodies are highly specific, being directed against a single antigenic site or multiple antigenic sites in the case of multispecific engineered antibodies. Furthermore, in contrast to polyclonal antibody preparations which include different antibodies directed against different determinants (epitopes), each monoclonal antibody is directed against the same determinant on the antigen. In addition to their specificity, the monoclonal antibodies are advantageous in that they may be synthesized uncontaminated by other antibodies.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art. The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. In some embodiments, an antibody herein is 90% or more monoclonal. Following is a description of representative methods for producing monoclonal antibodies which is not intended to be limiting and may be used to produce, for example, monoclonal mammalian, chimeric, humanized, human, domain, diabodies, vaccibodies, linear and multispecific antibodies.

Methods for producing and screening for specific antibodies using hybridoma technology are known in the art. Briefly, mice can be immunized with a target antigen (either the full length protein or a domain thereof, e.g., the extracellular domain or the ligand binding domain) and once an immune response is detected, e.g., antibodies specific for the target antigen are detected in the mouse serum, the mouse spleen is harvested and splenocytes isolated. The splenocytes are then fused by known techniques to any suitable myeloma cells, for example cells from cell line SP20 available from the ATCC. Hybridomas are selected and cloned by limited dilution. Hybridoma clones are then assayed by methods known in the art for cells that secrete antibodies capable of binding a polypeptide of an antibody herein. Ascites fluid, which generally contains high levels of antibodies, can be generated by immunizing mice with positive hybridoma clones.

Accordingly, monoclonal antibodies can be generated by culturing a hybridoma cell secreting an antibody herein, where the hybridoma is generated by fusing splenocytes isolated from a mouse immunized with a target antigen with myeloma cells and then screening the hybridomas resulting from the fusion for hybridoma clones that secrete an antibody able to bind to a specific target antigen.

Additionally, lymphocytes may be immunized in vitro. After immunization, lymphocytes are isolated and then fused with a myeloma cell line using a suitable fusing agent or fusion partner, such as polyethylene glycol, to form a hybridoma cell. In certain embodiments, the selected myeloma cells are those that fuse efficiently, support stable high-level production of antibody by the selected antibody-producing cells, and are sensitive to a selective medium that selects against the unfused parental cells. In one aspect, the myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Rockville, Md. USA. Human myeloma and mouse-human heteromyeloma cell lines for the production of human monoclonal antibodies are also known in the art.

Once hybridoma cells that produce antibodies of the desired specificity, affinity, and/or activity are identified, the clones may be subcloned by limiting dilution procedures and grown by standard methods. Suitable culture media for this purpose include, for example, D-MEM or RPMI-1640 medium. In addition, the hybridoma cells may be grown in vivo as ascites tumors in an animal, e.g, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, affinity tags, hydroxylapatite chromatography, gel electrophoresis, dialysis, etc. Exemplary purification methods are described in more detail below.

Antibody fragments which recognize specific target antigen epitopes may be generated by any technique known to those of skill in the art. For example, Fab and F(ab′)2 fragments herein may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies herein can also be generated using various phage display methods known in the art and more fully discussed below, including using antibody libraries derived from human immunoglobulin sequences.

### Antibody Mutation

Antibody mutation techniques are known in the art. In order to create a humanized antibody, for example, a vector for the production of a chimeric anti-murine antibodies lacking interchain cysteines may be constructed. Based, for example, on the sequence of anti-murine mAb, overlapping oligonucleotides encoding variable heavy chain and variable light chain domains (about 69-75 bases in length) may be synthesized and purified. The variable heavy and light domains may be synthesized separately by combining 25 pmol of each of the overlapping oligonucleotides with Pfu DNA polymerase (Stratagene) in a 50 .mu.l PCR reaction consisting of 5 cycles of: denaturing at 94.degree. C. for 20 sec, annealing at 50.degree. C. for 30 sec, ramping to 72.degree. C. over 1 min, and maintaining at 72.degree. C. for 30 sec. Subsequently, the annealing temperature can be increased to 55.degree. C. for 25 cycles. A reverse primer and a biotinylated forward primer may be used to further amplify 1 .mu.l of the fusion product in a 100 .mu.l PCR reaction using the same program. The products may be purified by agarose gel electrophoresis, electroeluted, and phosphorylated by T4 polynucleotide kinase (Boehringer Mannheim) and incubated with streptavidin magnetic beads (Boehringer Mannheim) in 5 mM Tris-Cl, pH 7.5, 0.5 mM EDTA, 1 M NaCl, and 0.05% Tween 20 for 15 min at 25.degree. C. The beads may be washed and the non-biotinylated, minus strand DNA eluted by incubating with 0.15 M NaOH at 25.degree. C. for 10 min. Chimeric anti-murine Fab may be synthesized, for example, in a modified M131×10⁴ phage vector, termed M131×10⁴CS, by hybridization mutagenesis using the variable heavy chain and variable light chain oligonucleotides in 3-fold molar excess of the uridinylated vector template. The M131×10⁴ vector may be modified by replacing cysteine residues at the end of the kappa and .gamma.1 constant regions with serine. The reaction may be electroporated into DH10B cells and titered onto a lawn of XL-1 Blue.

The anti-murine mAb variable region framework sequences are sometimes used to identify the most homologous human germline sequences. The heavy chain and light chain framework residues may be, for example, about 70% or more identical to the corresponding human germline sequences.

Specific residues may be substituted using site directed mutagenesis. PCR primer oligonucleotides may be designed to incorporate nucleotide changes to the coding sequence of the subject antibody that result in substitution of, for example, a cysteine residue for an amino acid at a specific position within the protein. A cysteine substitution mutation may be constructed by designing a primer to change, for example, the codon ACT for threonine at amino acid residue 135 to a TGT codon encoding cysteine. PCR may be performed in a 50 μl reaction in 1.times.PCR buffer (Perkin-Elmer buffer containing 1.5 mM MgCl.sub.2), 200 micromolar concentration of each of the four nucleotides dA, dC, dG and dT, with each oligonucleotide primer present at 0.5 .mu.M, 5 pg of template and 1.25 units of Amplitac DNA Polymerase (Perkin-Elmer) and 0.125 units of PFU DNA Polymerase (Stratagene). Reactions may be performed, for example, in a Robocycler Gradient 96 thermal cycler (Stratagene). The program may entailed: 95 deg C. for 3 minutes followed by 25 cycles of 95 deg C. for 60 seconds, 45 deg C. or 50 deg C. or 55 deg C. for 75 seconds, 72 deg C. for 60 seconds followed by a hold at 6 deg C. The PCR reactions may be analyzed by agarose gel electrophoresis to identify annealing temperatures that gave significant product of the expected size. The 45-deg C. reaction may be “cleaned up” using the QlAquick PCR Purification Kit (Qiagen), digested with appropriate enzymes. The resulting fragment, including the putative T135C mutation, may be gel-purified and ligated into an appropriate plasmid.

### Cysteine Substituted Antibodies

Antibodies herein include, in some embodiments, no interchain cysteines (e.g., they are replaced by non-thiol amino acids), certain amino acids substituted by cysteine, and combinations thereof.

### Cysteines Substituted by Non-Thiol Amino Acids

Interchain cysteines are replaced by non-thiol amino acids in some embodiments. In certain embodiments, interchain cysteine amino acids at positions shown in Table 1 independently are removed or substituted with an amino acid that is not cysteine. Such amino acids include, in some embodiments, naturally occurring and non-classical amino acid that lack a thiol moiety. Naturally occurring amino acids lacking a thiol moiety include glycine, alanine, valine, leucine, isoleucine, proline, serine, threonine, methionine, histidine, lysine, arginine, glutamate, aspartate, glutamine, asparagine, phenylalanine, tyrosine and tryptophan. Examples of non-classical amino acids include ornithine, diaminobutyric acid, norleucine, pyrylalanine, thienylalanine, naphthylalanine and phenylglycine. Other examples of non-classical amino acids are alpha and alpha-disubstituted amino acids, N-alkyl amino acids, lactic acid*, halide derivatives of natural amino acids such as trifluorotyrosine*, p-X-phenylalanine (where X is a halide such as F, Cl, Br, or O*, allylglycine*, 7-aminoheptanoic acid*, methionine sulfone*, norleucine*, norvaline*, p-nitrophenylalanine*, hydroxyproline#, thioproline*, methyl derivatives of phenylalanine (Phe) such as 4-methyl-Phe*, pentamethyl-Phe*, Phe (4-amino)#, Tyr (methyl)*, Phe (4-isopropyl)*, Tic (1,2,3,4-tetrahydroisoquinoline-3-carboxyl acid)*, diaminopropionic acid, Phe (4-benzyl)*, 4-aminobutyric acid (gamma-Abu)*, 2-aminobutyric acid (alpha-Abu)*, 6-aminohexanoic acid (epsilon-Ahx)*, 2-aminoisobutyric acid (Aib)*, 3-aminopropionic acid*, norvaline*, hydroxyproline, sarcosine, citrulline, homocitrulline, cysteic acid, t-butylglycine*, t-butylalanine*, phenylglycine*, cyclohexylalanine*, fluoroamino acids, designer amino acids such as beta-methyl amino acids, and the like. The notation * indicates a derivative having hydrophobic characteristics and # indicates a derivative having hydrophilic characteristics.

In certain embodiments, 1 to 8 interchain cysteines are replaced by valine (e.g., 2, 3, 4, 5, 6, 7 cysteines are replaced by valine. In some embodiments, amino acids at positions shown in Table 1 all are replaced by valine.

Antibody Amino Acids Substituted by Cysteine

In some embodiments, an antibody includes no native interchain cysteines and includes one or more non-cysteine amino acids substituted by cysteine. In some embodiments, 1, 2, 3, 4, 5, 6, 7, 8 or more amino acids of positions in the CH1 domain show in Table 2 are substituted with a cysteine amino acid, where the numbering system of the constant region is that of the EU index as set forth in Kabat et al. (1991, NTH Publication 91-3242, National Technical Information Service, Springfield, Va.). One or more or all of non-cysteine amino acids at positions shown in Table 2 may be replaced by cysteine. In some embodiments, such an antibody is a parent or wild-type antibody. A single substitution of a cysteine residue often results in the display of two cysteine residues in the resultant antibody due to the homodimeric nature of IgG molecules. The resultant antibodies lacking interchain cysteines herein may display at least 1, 2, 3, 4, 5, 6, 7, 8, 9, 10, 11, 12, 13, 14, 15, 16, 17, 18 or more free thiols for the purpose of conjugation to a heterologous molecule.

In some embodiments, antibodies herein comprise a serine shown at a position in Table 2 substituted to cysteine. In various embodiments, an antibody lacking interchain cysteines comprises a threonine shown at a position in Table 2 substituted to cysteine. In some embodiments, a cysteine engineered antibody herein comprises one or more serines and one or more threonines shown at positions in Table 2 substituted by cysteine. Such antibodies may include an IgG1, IgG2, IgG3 or IgG4 isotype heavy chain or portion thereof.

In some embodiments, an antibody lacking interchain cysteines comprises a cysteine substitution at one or more positions selected from positions 131, 132, 133, 134, 135, 136, 137, 138 and 139 shown in Table 2, where the numbering system of the constant region is that of the EU index as set forth in Kabat et al. (supra). In certain embodiments, a cysteine engineered antibody comprises a cysteine substitution at two or more positions selected from positions 131, 132, 133, 134, 135, 136, 137, 138 and 139 shown in Table 2. In certain embodiments, an antibody lacking interchain cysteines comprises cysteine substitutions at three or more positions selected from positions 131, 132, 133, 134, 135, 136, 137, 138 and 139 shown in Table 2. In various embodiments, an antibody lacking interchain cysteines comprises a cysteine substitution at four or more positions selected from positions 131, 132, 133, 134, 135, 136, 137, 138 and 139 shown in Table 2. In various embodiments, an antibody lacking interchain cysteines comprises a cysteine substitution at five or more positions selected from positions 131, 132, 133, 134, 135, 136, 137, 138 and 139 shown in Table 2. In some embodiments, an antibody lacking interchain cysteines comprises a cysteine substitution at six or more positions selected from positions 131, 132, 133, 134, 135, 136, 137, 138 and 139 shown in Table 2. In various embodiments, an antibody lacking interchain cysteines comprises a cysteine substitution at seven or more positions selected from positions 131, 132, 133, 134, 135, 136, 137, 138 and 139 shown in Table 2. In certain embodiments, an antibody lacking interchain cysteines comprises a cysteine substitution at eight or more positions selected from positions 131, 132, 133, 134, 135, 136, 137, 138 and 139 shown in Table 2. In various embodiments, an antibody lacking interchain cysteines comprises a cysteine substitution at nine or more positions selected from positions 131, 132, 133, 134, 135, 136, 137, 138 and 139 shown in Table 2. Positions in SEQ ID NOs: 5 to 10 corresponding to positions 131, 132, 133, 134, 135, 136, 137, 138 and 139 shown in Table 2 are determined by inspection.

A fragment of a full-length antibody may not include a full-length CH1 domain in certain embodiments. An antibody fragment may include one or more of the positions shown in Table 2, and one or more of the non-cysteine amino acids at such positions may be substituted by cysteine.

In some embodiments, an antibody lacking interchain cysteines does not comprise a substitution to at cystein positions 132 and/or 138 in Table 2. In some embodiments, an antibody lacking interchain cysteines positions 132 and/or 138 in Table 2 comprise a substitution that is not cysteine. In certain certain embodiments, an antibody lacking interchain cysteines comprises one or more substitutions at only threonine and/or serine amino acids naturally occurring at positions shown in Table 2, or equivalents thereof.

In an embodiment, an antibody lacking interchain cysteines includes an IgG1 having a serine and/or a threonine substituted for a cysteine at a position shown in Table 2. In various embodiments, antibodies lacking interchain cysteines herein are derived from an IgGI, IgG2, IgG3 or an IgG4 isotype. In certain embodiments, an antibody lacking interchain cysteines is derived from non-IgG formats such as FgAl, Ig A2, IgM, IgD, or IgE. In various embodiments, antibodies herein comprise one or more cysteine engineering of residues corresponding to one or more of positions 131-139 of IgG1 shown in Table 2. In some embodiments, antibodies herein comprise cysteine engineering of residues outlined in the various antibody formats. In some embodiments, antibodies herein comprise antibody fragments including, but not limited to Fab and Fab2 molecule formats.

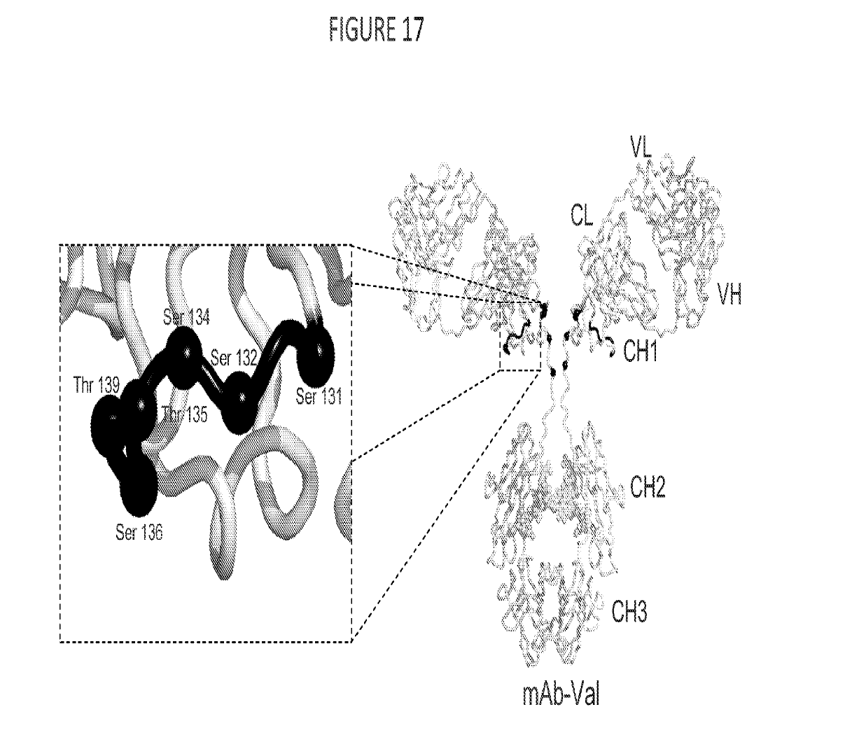

The 131-139 region of the CH1 domain of the IgG1 molecule is solvent exposed as illustrated in FIG. 17. As such, it is envisaged that the 131-139 loop may be expanded (in other words, inclusion of additional amino acids) to facilitate a surface for site-specific conjugation of various agents. In some embodiments, a loop shown in Table 2 of an antibody lacking native interchain cysteines is expanded by 1, 2, 3, 4, 5, 6, 7, 8, 9, 10, 11, 12, 13, 14, 15, at least 1, at least 2, at least 3, at least 4, at least 5, at least 6, at least 7, at least 8, at least 9, at least 10, at least 11, at least 12, at least 13, at least 14, or at least 15 amino acids. In some embodiments, an expansion of a loop shown in Table 2 occurs after one or more of the 131, 132, 133, 134, 135, 136, 137, 138, or 139 residue in an IgG1 isotype antibody or counterpart position in an IgG2, 3 or 4 isotype antibody. In various embodiments, an expansion of a loop shown in Table 2 occurs after the 131, 132, 133, 134, 135, 136, 137, 138, and 139 residue in an IgG1 isotype antibody or counterpart position in an IgG2, 3 or 4 isotype antibody.

In various embodiments, an expansion of a loop shown in Table 2 may comprise any amino acid, and in various embodiments, the expansion comprises at least one non-naturally occurring cysteine amino acid. In some embodiments, the expansion comprises threonine and/or serine residues, and in various embodiments, the expansion is also coupled with the substitution of non-naturally occurring cysteine residues for non-cysteine residues.

In certain embodiments, antibodies lacking interchain cysteines comprise the formation of at least one non-naturally occurring disulfide bond. The non-naturally occurring disulfide bond may be intrachain or interchain bond. The non-naturally occurring disulfide bond may link two separate antibody molecules together. The formation of a non-naturally occurring disulfide bond may liberate at least one free thiol group previously linked to another cysteine residue.

The engineering of cysteine residues to display free thiol groups may lead to a mixture of antibody species, displaying a high degree of variability of positions of disulfide bonds. For example, the naturally occurring “canonical” disulfide bond may only be represented in some of the antibodies present in a sample. It is understood that the engineering of other non-naturally occurring cysteines may lead to the formation of disulfide bonds other than the “canonical” disulfide bond. In some embodiments, a disulfide bond is formed between the light chain and any non-naturally occurring cysteine residue present in a loop shown in the Table 2. In certain embodiments, a disulfide bond is formed between the light chain and any non-naturally occurring cysteine residue present in a loop shown in the Table 2.

Also presented herein are antibodies lacking interchain cysteines where 1, 2, 3, 4, 5, 6, 7, 8, 9, 10, 11, 12, 13, 14, 15, 16, 17, 18, 19, 20, or more amino acids at positions selected from Table 3 are substituted by a cysteine amino acid. Thus, 1 to 20 amino acids at positions 239, 282, 289, 297, 312, 324, 330, 335, 337, 339, 356, 359, 361, 383, 384, 398, 400, 422, 440 in Table 3 of an antibody independently may be replaced with a cysteine amino acid, where the numbering system of the constant region is that of the EU index as set forth in Kabat et al. (1991, NIH Publication 91-3242, National Technical Information Service, Springfield, Va.) of a parent or wild type antibody are substituted with a cysteine amino acid. It should be noted that a single substitution of a cysteine residue results in the display of two cysteine residues in the resultant antibody due to the homodimeric nature of IgG molecules. The resultant antibodies lacking interchain cysteines herein may display at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more free thiols for the purpose of conjugation to a drug or compound. Such antibodies may include an IgG1, IgG2, IgG3 or IgG4 isotype heavy chain or portion thereof. Positions in SEQ ID NOs: 5 to 10 that correspond to positions 239, 282, 289, 297, 312, 324, 330, 335, 337, 339, 356, 359, 361, 383, 384, 398, 400, 422, 440 in Table 3 are determined by inspection.

In some embodiments, an antibody lacking interchain cysteines comprises a cysteine substitution at one or more positions selected from positions 239, 282, 289, 297, 312, 324, 330, 335, 337, 339, 356, 359, 361, 383, 384, 398, 400, 422, 440 in Table 3, where the numbering system of the constant region is that of the EU index as set forth in Kabat et al. (supra). In certain embodiments, an antibody lacking interchain cysteines comprises at least two substitutions selected from the positions 239, 282, 289, 297, 312, 324, 330, 335, 337, 339, 356, 359, 361, 383, 384, 398, 400, 422, 440 in Table 3. In various embodiments, an antibody lacking interchain cysteines comprises at least three substitutions selected from the positions 239, 282, 289, 297, 312, 324, 330, 335, 337, 339, 356, 359, 361, 383, 384, 398, 400, 422, 440 in Table 3. In some embodiments, an antibody lacking interchain cysteines comprises at least four substitutions selected from the positions 239, 282, 289, 297, 312, 324, 330, 335, 337, 339, 356, 359, 361, 383, 384, 398, 400, 422, 440 in Table 3. In certain embodiments, an antibody lacking interchain cysteines comprises at least five substitutions selected from the positions 239, 282, 289, 297, 312, 324, 330, 335, 337, 339, 356, 359, 361, 383, 384, 398, 400, 422, 440 in Table 3. In various embodiments, an antibody lacking interchain cysteines comprises at least six substitutions selected from the positions 239, 282, 289, 297, 312, 324, 330, 335, 337, 339, 356, 359, 361, 383, 384, 398, 400, 422, 440 in Table 3 of the heavy chain of an antibody. An antibody lacking interchain cysteines sometimes comprises at least seven substitutions selected from the positions 239, 282, 289, 297, 312, 324, 330, 335, 337, 339, 356, 359, 361, 383, 384, 398, 400, 422, 440 in Table 3. In some embodiments, an antibody lacking interchain cysteines comprises at least eight substitutions selected from the positions 239, 282, 289, 297, 312, 324, 330, 335, 337, 339, 356, 359, 361, 383, 384, 398, 400, 422, 440 in Table 3. In certain embodiments, an antibody lacking interchain cysteines comprises at least nine substitutions selected from the positions 239, 282, 289, 297, 312, 324, 330, 335, 337, 339, 356, 359, 361, 383, 384, 398, 400, 422, 440 in Table 3. In various embodiments, an antibody lacking interchain cysteines comprises at least ten substitutions selected from the positions 239, 282, 289, 297, 312, 324, 330, 335, 337, 339, 356, 359, 361, 383, 384, 398, 400, 422, 440 in Table 3. An antibody lacking interchain cysteines sometimes comprises at least ten substitutions selected from the positions 239, 282, 289, 297, 312, 324, 330, 335, 337, 339, 356, 359, 361, 383, 384, 398, 400, 422, 440 in Table 3. In some embodiments, an antibody lacking interchain cysteines comprises at least eleven substitutions selected from the positions 239, 282, 289, 297, 312, 324, 330, 335, 337, 339, 356, 359, 361, 383, 384, 398, 400, 422, 440 in Table 3. In certain embodiments, an antibody lacking interchain cysteines comprises at least twelve substitutions selected from the positions 239, 282, 289, 297, 312, 324, 330, 335, 337, 339, 356, 359, 361, 383, 384, 398, 400, 422, 440 in Table 3. In various embodiments, an antibody lacking interchain cysteines comprises at least thirteen substitutions selected from the positions 239, 282, 289, 297, 312, 324, 330, 335, 337, 339, 356, 359, 361, 383, 384, 398, 400, 422, 440 in Table 3. The cysteine antibodies lacking interchain cysteines herein sometimes comprise at least fourteen substitutions selected from the positions 239, 282, 289, 297, 312, 324, 330, 335, 337, 339, 356, 359, 361, 383, 384, 398, 400, 422, 440 in Table 3. In some embodiments, an antibody lacking interchain cysteines comprises at least fifteen substitutions selected from the positions 239, 282, 289, 297, 312, 324, 330, 335, 337, 339, 356, 359, 361, 383, 384, 398, 400, 422, 440 in Table 3. In certain embodiments, the antibodies lacking interchain cysteines antibodies herein comprise at least sixteen substitutions selected from the positions 239, 282, 289, 297, 312, 324, 330, 335, 337, 339, 356, 359, 361, 383, 384, 398, 400, 422, 440 in Table 3. In various embodiments, an antibody lacking interchain cysteines comprises at least seventeen substitutions selected from the positions 239, 282, 289, 297, 312, 324, 330, 335, 337, 339, 356, 359, 361, 383, 384, 398, 400, 422, 440 in Table 3. An antibody lacking interchain cysteines sometimes comprises at least eighteen substitutions selected from the positions 239, 282, 289, 297, 312, 324, 330, 335, 337, 339, 356, 359, 361, 383, 384, 398, 400, 422, 440 in Table 3. In some embodiments, an antibody lacking interchain cysteines comprises at least nineteen substitutions selected from the positions 239, 282, 289, 297, 312, 324, 330, 335, 337, 339, 356, 359, 361, 383, 384, 398, 400, 422, 440 in Table 3. In certain embodiments, an antibody lacking interchain cysteines comprises substitutions of the positions 239, 282, 289, 297, 312, 324, 330, 335, 337, 339, 356, 359, 361, 383, 384, 398, 400, 422, 440 in Table 3.

In some embodiments, an antibody lacking interchain cysteines comprises a substitution of at least one naturally occurring amino acid selected from the group consisting of: Ser239, VaI282, Thr289, Asn297, Asp312, Ser324, Ala330, Thr335, Ser337, Ala339, Glu356, Thr359, Asn361, Ser383, Asn384, Leu398, Ser400, Ser440, Va1422, and Ser442 of the heavy chain of an antibody based on IgG1, or counterpart position in an IgG2, IgG3 or IgG4 antibody (see Table 3). In certain embodiments, an antibody lacking interchain cysteines does not comprise a substitution at position or positions selected from: Ser239, Va1282, Thr289, Asn297, Asp312, Ser324, Ala330, Thr335, Ser337, Ala339, Glu356, Thr359, Asn361, Ser383, Asn384, Leu398, Ser400, Ser440, Va1422, and Ser442 of the heavy chain of an antibody based on IgG1, or counterpart position in an IgG2, IgG3 or IgG4 antibody (see Table 3).

In various embodiments, an antibody lacking interchain cysteines includes an IgG1 having a naturally occurring amino acid substituted for a cysteine at a position selected from the group consisting of: 239, 282, 289, 297, 312, 324, 330, 335, 337, 339, 356, 359, 361, 383, 384, 398, 400, 440, 422, and 442 of the heavy chain of an IgG1 antibody, or a counterpart position in an IgG2, IgG3 or IgG4 antibody. An antibody lacking interchain cysteines sometimes is derived from an IgG1, IgG2, IgG3 or an IgG4 format. In some embodiments, an antibody lacking interchain cysteines is derived from non-IgG formats such as IgA1, IgA2 IgM, IgD, or IgE. In certain embodiments, antibodies herein comprise cysteine engineering of surface residues of the CH2 and/or CH3 region of an IgG1 molecule or equivalents thereof.

A fragment of a full-length antibody may not include a full-length CH2 domain and/or full-length CH3 domain, in certain embodiments. An antibody fragment may include one or more of the positions shown in Table 3, and one or more of the amino acids at such positions may be substituted by cysteine.

In various embodiments, an antibody herein comprises the expression of an isolated Fc region comprising residues of antibody lacking interchain cysteines. Such isolated Fc regions may be useful as scaffolds for display purposes.

In certain embodiments, provided herein are fusion proteins comprising Fc regions that contain at least one or more substitutions at positions selected from positions 239, 282, 289, 297, 312, 324, 330, 335, 337, 339, 356, 359, 361, 383, 384, 398, 400, 422, 440 in Table 3.

In some embodiments, engineered antibodies herein may comprise one or more non-naturally occurring cysteine amino acids in the loop shown in Table 2 and one or more non-naturally occurring cysteine amino acids at position shown in Table 3.

### Fc Region

Provided herein, in some embodiments, are antibodies lacking native interchain cysteines with modifications to Fc regions described above (e.g., substitution of non-cysteine positions shown in Table 3 by cysteine amino acids). Also provided are antibodies lacking native interchain cysteines with one or more other Fc modifications described hereafter. Thus, in some embodiments an antibody lacking interchain cysteines may have an amino acid sequence having at least 25%, 35%, 45%, 55%, 65%, 75%, 80%, 85%, 90%, 95%, 96%, 97%, 98%, or 99% amino acid sequence identity or similarity with the amino acid sequence of the Fc region of a parent antibody.

Certain modifications to the Fc region (e.g., amino acid substitutions and/or additions and/or deletions) can enhance or diminish effector function. In certain embodiments, variant Fc regions of antibodies exhibit a similar level of inducing effector function as compared to native Fc. In various embodiments, a variant Fc region exhibits a higher induction of effector function as compared to the native Fc. A variant Fc region sometimes exhibits lower induction of effector function as compared to the native Fc. In some embodiments, a variant Fc region exhibits higher induction of ADCC as compared to the native Fc. In certain embodiments, a variant Fc region exhibits lower induction of ADCC as compared to the native Fc. In some embodiments, a variant Fc region exhibits higher induction of CDC as compared to the native Fc. In some embodiments, a variant Fc region exhibits lower induction of CDC as compared to the native Fc.

In addition, glycosylation of a Fc region can be modified to increase or decrease effector function. In some embodiments the cysteine engineering creates a glycosylation site not present in an antibody counterpart having a native interchain cysteine amino acid. Accordingly, in some embodiments, the Fc regions of antibodies herein comprise altered glycosylation of amino acid residues. In certain embodiments, the altered glycosylation of the amino acid residues results in lowered effector function. In various embodiments, the altered glycosylation of the amino acid residues results in increased effector function. In various embodiments, the Fc region has reduced glycosylation. In some embodiments, the Fc region is glycosylated.

The addition of sialic acid to the oligosaccharides on IgG molecules may enhances their anti-inflammatory activity and alter their cytotoxicity. Thus, the efficacy of antibody therapeutics may be optimized by selection of a glycoform that is best suited to the intended application. The two oligosaccharide chains interposed between the two CH2 domains of antibodies are involved in the binding of the Fc region to its receptors. IgG molecules with increased sialylation exhibit anti-inflammatory properties whereas IgG molecules with reduced sialylation show increased immunostimulatory properties. Therefore, an antibody therapeutic can be “tailor-made” with an appropriate sialylation profile for a particular application. Methods for modulating the sialylation state of antibodies are known in the art.

In some embodiments, the Fc regions of antibodies herein comprise an altered sialylation profile compared to a reference unaltered Fc region. In certain embodiments, the Fe regions of antibodies herein comprises an increased sialylation profile compared to a reference unaltered Fc region. In various embodiments the Fc regions of antibodies herein comprise an increase in sialylation of about 5%, about 10%, about 15%, about 20%, about 25%, about 30%, about 35%, about 40%, about 45%, about 50%, about 60%, about 65%, about 70%, about 80%, about 85%, about 90%, about 95%, about 100%, about 125%, about 150% or more as compared to a reference unaltered Fc region. In some embodiments the Fc regions of antibodies herein comprise an increase in sialylation of about 2 fold, about 3 fold, about 4 fold, about 5 fold, about 10 fold, about 20 fold, about 50 fold or more as compared to an unaltered reference Fc region.

In some embodiments, the Fc regions of antibodies herein comprise a decreased sialylation profile compared to a reference unaltered Fc region. In some embodiments, the Fc regions of antibodies herein comprise a decrease in sialylation of about 5%, about 10%, about 15%, about 20%, about 25%, about 30%, about 35%, about 40%, about 45%, about 50%, about 60%, about 65%, about 70%, about 80%, about 85%, about 90%, about 95%, about 100%, about 125%, about 150% or more as compared to a reference unaltered Fc region. In some embodiments the Fc regions of antibodies herein comprise a decrease in sialylation of about 2 fold, about 3 fold, about 4 fold, about 5 fold, about 10 fold, about 20 fold, about 50 fold or more as compared to an unaltered reference Fc region.

The Fc region can also be modified to increase the half-lives of proteins. The increase in half-life allows for the reduction in amount of drug given to a patient as well as reducing the frequency of administration. Accordingly, antibodies herein with increased half-lives may be generated by modifying (for example, substituting, deleting, or adding) amino acid residues identified as involved in the interaction between the Fc and the FcRn receptor. In certain embodiments, a methionine at position 252, a serine at position 254 and a threonine at position 256 of an IgG1 isotype antibody can be changed to tyrosine, threonine and glutamate, respectively, such that the resulting antibody includes tyrosine-252, threonine-254 and glutamate-256. Such an Fc region of an IgG1 antibody includes a YTE modification and counterpart positions in can be similarly modified in IgG2, IgG3 and IgG4 antibodies. In addition, the half-life of antibodies herein may be increase by conjugation to PEG or Albumin by techniques known in the art. In some embodiments the Fc regions of antibodies herein comprise an increase in half-life of about 5%, about 10%, about 15%, about 20%, about 25%, about 30%, about 35%, about 40%, about 45%, about 50%, about 60%, about 65%, about 70%, about 80%, about 85%, about 90%, about 95%, about 100%, about 125% s, about 150% or more as compared to a reference unaltered Fc region. In certain embodiments the Fc regions of antibodies herein comprise an increase in half-life of about 2 fold, about 3 fold, about 4 fold, about 5 fold, about 10 fold, about 20 fold, about 50 fold or more as compared to an unaltered reference Fc region.

In various embodiments, the Fc regions of antibodies herein comprise a decrease in half-life. In some embodiments the Fc regions of antibodies herein comprise a decrease in half-life of about 5%, about 10%, about 15%, about 20%, about 25%, about 30%, about 35%, about 40%, about 45%, about 50%, about 60%, about 65%, about 70%, about 80%, about 85%, about 90%, about 95%, about 100%, about 125%, about 150% or more as compared to a reference unaltered Fc region. In some embodiments the Fc regions of antibodies herein comprise a decrease in half-life of about 2 fold, about 3 fold, about 4 fold, about 5 fold, about 10 fold, about 20 fold, about 50 fold or more as compared to an unaltered reference Fc region.

Also presented herein, in certain embodiments, are Fc variant proteins which have altered binding properties for an Fc ligand (e.g., an Fc receptor, Clq) relative to a comparable molecule (e.g., a protein having the same amino acid sequence except having a wild type Fc region). Examples of binding properties include but are not limited to, binding specificity, equilibrium dissociation constant (K_{D}), dissociation and association rates (k_{off} and kₒₙ respectively), binding affinity and/or avidity, It is generally understood that a binding molecule (e.g., a Fc variant protein such as an antibody) with a low Kₒₙ may be preferable to a binding molecule with a high k_{off}. However, in some instances the value of the kₒₙ or k_{off} may be more relevant than the value of the K_{D}. One skilled in the art can determine which kinetic parameter is most important for a given antibody application.

The affinities and binding properties of an Fc region for its ligand may be determined by a variety of in vitro assay methods (biochemical or immunological based assays) known in the art for determining Fc-FcγR interactions, i.e., specific binding of an Fc region to an FcR including but not limited to, equilibrium methods (e.g., enzyme-linked immunoabsorbent assay (ELISA), or radioimmunoassay (RIA)), or kinetics (e.g., BIACORE® analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods including but not limited to colorimetric, spectrometric, spectrophotometic, fluorescent, luminescent, or isotopic labels.

In some embodiments, the Fc variant protein has enhanced binding to one or more Fc ligand relative to a comparable molecule. In certain embodiments, the Fc variant protein has an affinity for an Fc ligand that is at least 2 fold, at least 3 fold, at least 5 fold, at least 7 fold, at least 10 fold, at least 20 fold, at least 30 fold, at least 40 fold, at least 50 fold, at least 60 fold, at least 70 fold, at least 80 fold, at least 90 fold, at least 100 fold, or at least 200 fold greater than that of a comparable molecule. In various embodiments, the Fc variant protein has enhanced binding to an Fc receptor. In some embodiments, the Fc variant protein has enhanced binding to the Fc receptor FcγRIIIA. In certain embodiments, the Fc variant protein has enhanced biding to the Fc receptor FcγR ITB, In various embodiments, the Fc variant protein has enhanced binding to the Fc receptor FcRn. The Fc variant protein sometimes has enhanced binding to Clq relative to a comparable molecule.

The ability of any particular Fc variant protein to mediate lysis of the target cell by ADCC can be assayed. To assess ADCC activity an Fc variant protein of interest is added to target cells in combination with immune effector cells, which may be activated by the antigen antibody complexes resulting in cytolysis of the target cell. Cytolysis is generally detected by the release of label (e.g. radioactive substrates, fluorescent dyes or natural intracellular proteins) from the lysed cells. Effector cells for such assays may include peripheral blood mononuclear cells (PBMC) and Natural Killer (NK) cells. Specific examples of in vitro ADCC assays are known in the art.

In some embodiments, an Fc variant protein has enhanced ADCC activity relative to a comparable molecule. In specific embodiments, an Fc variant protein has ADCC activity that is at least 2 fold, or at least 3 fold, at least 5 fold, at least 10 fold, at least 50 fold, or at least 100 fold greater than that of a comparable molecule. In certain embodiments, an Fc variant protein has enhanced binding to the Fc receptor FcγRIIIA and has enhanced ADCC activity relative to a comparable molecule. In some embodiments, the Fc variant protein has both enhanced ADCC activity and enhanced serum half life relative to a comparable molecule.

In certain embodiments, an Fc variant protein has reduced ADCC activity relative to a comparable molecule. In some embodiments, an Fc variant protein has ADCC activity that is at least 2 fold, at least 3 fold, at least 5 fold, at least 10 fold, at least 50 fold, or at least 100 fold lower than that of a comparable molecule. In various embodiments, an Fc variant protein has reduced binding to the Fe receptor FcγRIIIA and has reduced ADCC activity relative to a comparable molecule. The Fc variant protein sometimes has both reduced ADCC activity and enhanced serum half life relative to a comparable molecule.

In an embodiment, an Fc variant protein has enhanced CDC activity relative to a comparable molecule. In some embodiments, an Fc variant protein has CDC activity that is at least 2 fold, at least 3 fold, at least 5 fold, at least 10 fold, at least 50 fold, or at least 100 fold greater than that of a comparable molecule. In certain embodiments, the Fc variant protein has both enhanced CDC activity and enhanced serum half life relative to a comparable molecule. In an embodiment, the Fc variant protein has reduced binding to one or more Fc ligand relative to a comparable molecule. In some embodiments, the Fc variant protein has an affinity for an Fc ligand that is at least 2 fold, at least 3 fold, at least 5 fold, at least 7 fold, at least 10 fold, at least 20 fold, at least 30 fold, at least 40 fold, at least 50 fold, at least 60 fold, at least 70 fold, at least 80 fold, at least 90 fold, at least 100 fold, or at least 200 fold lower than that of a comparable molecule.

In some embodiments, the Fc variant protein has reduced binding to an Fc receptor. In certain embodiments, the Fc variant protein has reduced binding to the Fc receptor FcγRIIIA. In various embodiments, an Fc variant described herein has an affinity for the Fc receptor FcγRIIIA that is at least about 5 fold lower than that of a comparable molecule, where said Fc variant has an affinity for the be receptor FcγRIIB that is within about 2 fold of that of a comparable molecule. The Fc variant protein sometimes has reduced binding to the Fc receptor FcRn. In some embodiments, the Fc variant protein has reduced binding to Clq relative to a comparable molecule.

In some embodiments of Fc variants the Fc region comprises a non naturally occurring amino acid residue at one or more positions selected from the group consisting of 234, 235, 236, 237, 238, 239, 240, 241, 243, 244, 245, 247, 251, 252, 254, 255, 256, 262, 263, 264, 265, 266, 267, 268, 269, 279, 280, 284, 292, 296, 297, 298, 299, 305, 313, 316, 325, 326, 327, 328, 329, 330, 331, 332, 333, 334, 339, 341, 343, 370, 373, 378, 392, 416, 419, 421, 440 and 443 as numbered by the EU index as set forth in Kabat.

Optionally, the Fc region may comprise a non-naturally occurring amino acid residue at additional and/or alternative positions known to one skilled in the art. Provided, in some embodiments, is an Fc variant, where the Fc region comprises at least one non naturally occurring amino acid residue selected from the group consisting of 234D, 234E, 234N, 234Q, 234T, 234H, 234Y, 2341, 234V, 234F, 235A, 235D, 235R, 235W, 235P, 235S, 235N, 235Q, 235T, 235H, 235Y, 2351, 235V, 235F, 236E, 239D, 239E, 239N, 239Q, 239F, 239T, 239H, 239Y, 2401, 240A, 240T, 240M, 241W, 241 L, 241Y, 241E, 241 R. 243W, 243L 243Y, 243R, 243Q, 244H, 245A, 247L, 247V, 247G, 251 F, 252Y, 254T, 255L, 256E, 256M, 2621, 262A, 262T, 262E, 2631, 263A, 263T, 263M, 264L, 2641, 264W, 264T, 264R, 264F, 264M, 264Y, 264E, 265G, 265N, 265Q, 265Y, 265F, 265V, 2651, 265L, 265H, 265T, 2661, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 2961, 296H, 269G, 297S, 297D, 297E, 298H, 298I, 298T, 298F, 2991, 299L, 299A, 299S, 299V, 299H, 299F, 299E, 3051, 313F, 316D, 325Q, 325L, 3251, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 3301, 330F, 330R, 330H, 331G, 331A, 331L, 331M, 331F, 331W, 331K, 331Q, 331E, 331S, 331V, 3311, 331C, 331Y, 331H, 331R, 331N, 331D, 331T, 332D, 332S, 332W, 332F, 332E, 332N, 332Q, 332T, 332H, 332Y, 332A, 339T, 370E, 370N, 378D, 392T, 396L, 416G, 419H, 421K, 440Y and 443W as numbered by the EU index as set forth in Kabat. Optionally, the Fc region may comprise additional and/or alternative non naturally occurring amino acid residues known in the art.

### Certain Amino Acid Sequence Modifications

In certain embodiments, the amino acid sequence of a light chain in an antibody lacking interchain cysteines is about 80% or more identical to the amino acid sequence of SEQ ID NO: 9 or 10 (e.g., about 85% or more, 86% or more, 87% or more, 88% or more, 89% or more, 90% or more, 91% or more, 92% or more, 93% or more, 94% or more, 95% or more, 96% or more, 97% or more, 98% or more, 99% or more identical to the amino acid sequence of SEQ ID NO: 9 or 10). In some embodiments, the amino acid sequence of a light chain is identical to the amino acid sequence in SEQ ID NO: 9 or 10 except that it includes 1, 2, 3, 4, 5, 6, 7, 8, 9 or 10 amino acid modifications (e.g., substitutions, insertions and/or deletions) relative to the amino acid sequence in SEQ ID NO: 9 or 10. In some embodiments, the amino acid sequence of a heavy chain is about 80% or more identical to the amino acid sequence of SEQ ID NO: 5, 6, 7 or 8 (e.g., about 85% or more, 86% or more, 87% or more, 88% or more, 89% or more, 90% or more, 91% or more, 92% or more, 93% or more, 94% or more, 95% or more, 96% or more, 97% or more, 98% or more, 99% or more identical to the amino acid sequence of SEQ ID NO: 5, 6, 7 or 8). In some embodiments, the amino acid sequence of a heavy chain is identical to the amino acid sequence in SEQ ID NO: 5, 6, 7 or 8 except that it includes 1, 2, 3, 4, 5, 6, 7, 8, 9 or 10 amino acid modifications (e.g., substitutions, insertions and/or deletions) relative to the amino acid sequence in SEQ ID NO: 5, 6, 7 or 8. As described herein, in some embodiments, a light chain and/or a variable chain sequence can include 1 to 10 engineered cysteine substitutions of non-cysteine amino acids.

In addition to the sequences of SEQ ID NOs: 1, 2, 3, 4, 5, 6, 7, 8, 9 or 10, the present technology also encompasses further modifications and, the variants and fragments thereof, comprising one or more amino acid residues and/or polypeptide substitutions, additions and/or deletions in the variable light (VL) domain and/or variable heavy (VH) domain and/or Fc region and post translational modifications. Included in these modifications are antibody conjugates where an antibody has been covalently attached to a moiety. Moieties suitable for attachment to the antibodies include but are not limited to, proteins, peptides, drugs, labels, and cytotoxins. These changes to the antibodies may be made to alter or fine tune the characteristics (biochemical, binding and/or functional) of the antibodies as is appropriate for treatment and/or diagnosis of antigen mediated diseases. Methods for forming conjugates, making amino acid and/or polypeptide changes and post-translational modifications are known in the art, some of which are detailed below. The following description is not intended to be limiting, but instead a non-limiting description of some embodiments, more of which will be understood by one of skill in the art. It is also understood that some of the following methods were used to develop the human, humanized and/or chimeric antibody sequences described above. Any combination of deletion, insertion, and substitution is made to arrive at the final construct, provided that the final construct possesses the desired characteristics.

Amino acid changes to the antibodies necessarily results in sequences that are less than 100% identical to the above identified antibody sequences or parent antibody sequence. In certain embodiments, in this context, the antibodies herein many have about 25% to about 95% sequence identity to the amino acid sequence of either the heavy or light chain variable domain of a parent antibody. Thus, in some embodiments an antibody lacking interchain cysteines may have an amino acid sequence having at least 25%, 35%, 45%, 55%, 65%, 75%, 80%, 85%, 90%, or 95% amino acid sequence identity or similarity with the amino acid sequence of either the heavy or light chain variable domain of a parent antibody. In certain embodiments, an antibody lacking interchain cysteines has an amino acid sequence having at least 25%, 35%, 45%, 55%, 65%, 75%, 80%, 85%, 90%, or 95% amino acid sequence identity or similarity with the amino acid sequence of the heavy or light chain CDR1, CDR2, or CDR3 of a parent antibody. An antibody lacking interchain cysteines may sometimes have an amino acid sequence having at least 25%, 35%, 45%, 55%, 65%, 75%, 80%, 85%, 90%, or 95% amino acid sequence identity or similarity with the amino acid sequence of the heavy or light chain FR1, FR2, FR3 or FR4 of a parent antibody.

In certain embodiments, altered antibodies are generated by one or more amino acid alterations (e.g., substitutions, deletion and/or additions) introduced in one or more of the variable regions of the antibody. In various embodiments, the amino acid alterations are introduced in the framework regions. One or more alterations of framework region residues may result in an improvement in the binding affinity of the antibody for the antigen. This may be especially true when these changes are made to humanized antibodies where the framework region may be from a different species than the CDR regions. Examples of framework region residues to modify include those which non-covalently bind antigen directly, interact with/effect the conformation of a CDR, and/or participate in the VL-VH interface. In some embodiments, from about one to about five framework residues may be altered. Sometimes, this may be sufficient to yield an antibody mutant suitable for use in preclinical trials, even where none of the hypervariable region residues have been altered. Normally, however, an altered antibody may comprise additional hypervariable region alteration(s). In certain embodiments, the hypervariable region residues may be changed randomly, especially where the starting binding affinity of an antibody lacking interchain cysteines for the antigen from the second mammalian species is such that such randomly produced antibodies can be readily screened.

### Specific Binding Activity

Antibodies lacking interchain cysteines herein retain the antigen binding capability of their wild type counterpart. In an embodiment, an antibody lacking interchain cysteines exhibits essentially the same affinity as compared to an antibody prior to cysteine engineering. In some embodiments, antibodies lacking interchain cysteines herein exhibit a reduced affinity as compared to an antibody prior to cysteine engineering. In some embodiments, antibodies lacking interchain cysteines herein exhibit an enhanced affinity as compared to an antibody prior to cysteine engineering.

An antibody herein may have a high binding affinity to one or more of its cognate antigens. For example, an antibody described herein may have an association rate constant or kₒₙ rate (antibody (Ab)+antigen->Ab-Ag) of at least 2×10⁵ M⁻¹s⁻¹, at least 5×10⁵ M⁻¹s⁻¹, at least 10⁶ M⁻¹s⁻¹, at least 5×10⁶ M⁻¹s¹, at least 10⁷ M⁻¹s⁻¹, at least 5×10⁷ M⁻¹s⁻¹, or at least 10⁸ M⁻¹s⁻¹.

In some embodiments, an antibody may have a k_{off} rate (Ab−Ag→Ab+Ag) of less than 5×10⁻¹ s⁻¹, less than 10⁻¹ s⁻¹, less than 5×10⁻² s⁻¹, less than 10⁻² s⁻¹, less than 5×10⁻³ s⁻¹, less than 10⁻³ s⁻¹, less than 5×10⁻⁴ s⁻¹, or less than 10⁻⁴ s⁻¹. In a some embodiments, an antibody herein has a k_{off} of less than 5×10⁻⁵ s⁻¹, less than 10⁻⁵ s⁻¹, less than 5×10⁻⁶ s⁻¹, less than 10⁻⁶ s⁻¹, less than 5×10⁻⁷ s⁻¹, less than 10⁻⁷ s⁻¹, less than 5×10⁻⁸ s⁻¹, less than 10⁻⁸ s⁻¹, less than 5×10⁻⁹ s⁻¹, less than 10⁻⁹ s⁻¹, or less than 10⁻¹⁰s⁻¹.

In some embodiments, an antibody may have an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹.

In certain embodiments, an antibody may have a dissociation constant or K_{d} (k_{off}/kₒₙ) of less than 5×10⁻² M, less than 10⁻² M, less than 5×10⁻³ M, less than 10⁻³ M, less than 5×10⁻⁴ M, less than 10⁻⁴ M, less than 5×10⁻⁵ M, less than 10⁻⁵ M, less than 5×10⁻⁶ M, less than 10⁻⁶ M, less than 5×10⁻⁷ M, less than 10⁻⁷M, less than 5×10⁻⁸ M, less than 10⁻⁸ M, less than 5×10⁻⁹ M, less than 10⁻⁹ M, less than 5×10⁻¹⁰ M, less than 10⁻¹ M, less than 5×10⁻¹¹ M, less than 10⁻¹¹M, less than 5×10⁻¹² M, less than 10⁻¹² M, less than 5×10⁻¹³ M, less than 10⁻¹³ M, less than 5×10⁻¹⁴ M, less than 10⁻¹⁴ M, less than 5×10⁻¹⁵ M, or less than 10⁻¹⁵ M.

An antibody used in accordance with a method described herein may have a dissociation constant (K_{d}) of less than 3000 pM, less than 2500 pM, less than 2000 pM, less than 1500 pM, less than 1000 pM, less than 750 pM, less than 500 pM, less than 250 pM, less than 200 pM, less than 150 pM, less than 100 pM, less than 75 pM as assessed using a method described herein or known to one of skill in the art (e.g., a BIAcore assay, ELISA) (Biacore International AB, Uppsala, Sweden).

In some embodiments, antibodies herein comprise an epitope binding domain that competes for binding with another antibody. In certain embodiments, antibodies lacking interchain cysteines herein comprise an epitope binding domain that specifically binds to an antigen selected from: PDGFRalpha, PDGFRbeta, PDGF, VEGF, VEGF-A, VEGF-B, VEGF-C. VEGF-D, VEGFE, VEGF-F, VEGFR-1, VEGFR-2, VEGFR-3, FGF, FGF2, HGF, KDR, flt-1, FLK-1 Ang-2, Ang-1, PLGF, CEA, CXCL13, Baff, IL-21, CCL21, TNF-alpha, CXCL12, SDF-1, bFGF, MAC-1, IL23p19, FPR, IGFBP4, CXCR3, TLR4, CXCR2, EphA2, EphA4, EphrinB2, EGFR(ErbB1), HER2(ErbB2 or p185neu), HER3(ErbB3), HER4 ErbB4 or tyro2), SC1, LRP5, LRP6, RAGE, Nav1.7, GLP1, RSV, RSV F protein, Influenza HA protein, Influenza NA protein, HMGB1, CD16, CD19, CD20, CD21, CD28, CD32, CD32b, CD64, CD79, CD22, ICAM-1, FGFR1, FGFR2, HDGF, EphB4, GITR, β-amyloid, hMPV, PIV-1, PIV-2, OX40L, IGFBP3, cMet, PD-1, PLGF, Neprolysin, CTD, IL-18, IL-6, CXCL-13, IL-1R1, IL-15, IL-4R, IgE, PAI-1, NGF, EphA2, CEA, uPARt, DLL-4, αvβ6, α5β1, interferon receptor type I and type II. CD19, ICOS, IL-17, Factor II, Hsp90, IGF, CD19, GM-CSFR, PIV-3, CMV, IL-13, IL-9, and EBV.

In various embodiments, an antibody lacking interchain cysteines comprises at least one epitope binding domain that specifically binds to a member (receptor or ligand) of the TNF superfamily. Various molecules include, but are not limited to Tumor Necrosis Factor-alpha (“TNF-alpha”), Tumor Necrosis Factor-beta (“TNF-beta”), Lymphotoxin-alpha (“LT-alpha”), CD30 ligand, CD27 ligand, CD40 ligand, 4-1 BB ligand, Apo-1 ligand (also referred to as Fas ligand or CD95 ligand), Apo-2 ligand (also referred to as TRAIL), Apo-3 ligand (also referred to as TWEAK), osteoprotegerin (OPG), APRIL, RANK ligand (also referred to as TRANCE), TALL-1 (also referred to as BlyS, BAFF or THANK), DR4, DR5 (also known as Apo-2, TRAIL-R2, TR6, Tango-63, hAPO8, TRICK2, or KILLER), DR6, DcR1, DcR2, DcR3 (also known as TR6 or M68), CAR1, HVEM (also known as ATAR or TR2), GITR, ZTNFR-5, NTR-1, TNFL1, CD30, LTBr, 4-1 BB receptor and TR9.

In some embodiments the antibodies lacking interchain cysteines herein are capable of binding one or more targets selected from the group consisting of 5T4, ABL, ABCF1, ACVR1, ACVR1B, ACVR2, ACVR2B, ACVRL1, ADORA2A, Aggrecan, AGR2, AICDA, AIF1, AIGI, AKAP1, AKAP2, AMH, AMHR2, ANGPT1, ANGPT2, ANGPTL3, ANGPTL4, ANPEP, APC, APOC1, AR, aromatase, ATX, AX1, AZGP1 (zinc-a-glycoprotein), B7.1, B7.2, B7-H1, BAD, BAFF, BAG1, BAI1, BCR, BCL2, BCL6, BDNF, BLNK, BLR1 (MDR15), BlyS, BMP1, BMP2, BMP3B (GDFIO), BMP4, BMP6, BMP8, BMPR1A, BMPR1B, BMPR2, BPAG1 (plectin), BRCA1, C19orf10 (IL27w), C3, C4A, C5, C5R1, CANT1, CASP1, CASP4, CAV1, CCBP2 (D6/JAB61), CCL1 (1-309), CCLI1 (eotaxin), CCL13 (MCP-4), CCL15 (MIP-Id), CCL16 (HCC-4), CCL17 (TARC), CCL18 (PARC), CCL19 (MIP-3b), CCL2 (MCP-1), MCAF, CCL20 (MIP-3a), CCL21 (MEP-2), SLC, exodus-2, CCL22(MDC/STC-I), CCL23 (MPIF-I), CCL24 (MPIF-2/eotaxin-2), CCL25 (TECK), CCL26(eotaxin-3), CCL27 (CTACK/ILC), CCL28, CCL3 (MIP-la), CCL4 (MIPIb), CCL5(RANTES), CCL7 (MCP-3), CCL8 (mcp-2), CCNA1, CCNA2, CCND1, CCNE1, CCNE2, CCRI (CKR1/HM145), CCR2 (mcp-IRB/RA), CCR3 (CKR3/CMKBR3), CCR4, CCR5(CMKBR5/ChemR13), CCR6 (CMKBR6/CKR-L3/STRL22/DRY6), CCR7 (CKR7/EBI1), CCR8 (CMKBR8/TERI/CKR-L1), CCR9 (GPR-9-6), CCRL1 (VSHK1), CCRL2 (L-CCR), CD164, CD19, CDIC, CD20, CD200, CD-22, CD24, CD28, CD3, CD33, CD35, CD37, CD38, CD3E, CD3G, CD3Z, CD4, CD40, CD40L, CD44, CD45RB, CD52, CD69, CD72, CD74, CD79A, CD79B, CD8, CD80, CD81, CD83, CD86, CD137, CDH1 (Ecadherin), CDH1O, CDH12, CDH13, CDH18, CDH19, CDH20, CDH5, CDH7, CDH8, CDH9, CDK2, CDK3, CDK4, CDK5, CDK6, CDK7, CDK9, CDKN1A (p21Wap1/Cipl), CDKN1B (p27Kipl), CDKNIC, CDKN2A (p16INK4a), CDKN2B, CDKN2C, CDKN3, CEBPB, CERI, CHGA, CHGB, Chitinase, CHST1O, CKLFSF2, CKLFSF3, CKLFSF4, CKLFSF5, CKLFSF6, CKLFSF7, CKLFSF8, CLDN3, CLDN7 (claudin-7), CLN3, CLU (clusterin), CMKLR1, CMKOR1 (RDC1), CNR1, COL18A1, COLIA1, COL4A3, COL6A1, CR2, Cripto, CRP, CSF1 (M-CSF), CSF2 (GM-CSF), CSF3 (GCSF), CTLA4, CTL8, CTNNB1 (b-catenin), CTSB (cathepsin B), CX3CL1 (SCYD1), CX3CR1 (V28), CXCL1 (GRO1), CXCL10 (IP-10), CXCLI1 (I-TAC/IP-9), CXCL12 (SDF1), CXCL13, CXCL14, CXCL16, CXCL2 (GRO2), CXCL3 (GRO3), CXCL5 (ENA-78/LIX), CXCL6 (GCP-2), CXCL9 (MIG), CXCR3 (GPR9/CKR-L2), CXCR4, CXCR6 (TYMSTR/STRL33/Bonzo), CYB5, CYC1, CYSLTRI, DAB2IP, DES, DKFZp451J0118, DNCL1, DPP4, E2F1, Engel, Edge, Fennel, EFNA3, EFNB2, EGF, EGFR, ELAC2, ENG, Enola, ENO2, ENO3, EPHA1, EPHA2, EPHA3, EPHA4, EPHA5, EPHA6, EPHA7, EPHA8, EPHA9, EPHA10, EPHB1, EPHB2, EPHB3, EPHB4, EPHB5, EPHB6, EPHRIN-A1, EPHRIN-A2, EPHRINA3, EPHRIN-A4, EPHRIN-A5, EPHRIN-A6, EPHRIN-B1, EPHRIN-B2, EPHRIN-B3, EPHB4, EPG, ERBB2 (Her-2), EREG, ERK8, Estrogen receptor, Earl, ESR2, F3 (TF), FADD, farnesyltransferase, FasL, FASNf, FCERIA, FCER2, FCGR3A, FGF, FGF1 (aFGF), FGF1O, FGF1 1, FGF12, FGF12B, FGF13, FGF14, FGF16, FGF17, FGF18, FGF19, FGF2 (bFGF), FGF20, FGF21, FGF22, FGF23, FGF3 (int-2), FGF4 (HST), FGF5, FGF6 (HST-2), FGF7 (KGF), FGF8, FGF9, FGFR3, FIGF (VEGFD), FIL1 (EPSILON), FBL1 (ZETA), FLJ12584, FLJ25530, FLRT1 (fibronectin), FLT1, FLT-3, FOS, FOSL1(FRA-1), FY (DARC), GABRP (GABAa), GAGEBI, GAGEC1, GALNAC4S-6ST, GATA3, GD2, GDF5, GFI1, GGT1, GM-CSF, GNAS1, GNRH1, GPR2 (CCR1O), GPR31, GPR44, GPR81 (FKSG80), GRCC1O (C1O), GRP, GSN (Gelsolin), GSTP1, HAVCR2, HDAC, HDAC4, HDAC5, HDAC7A, HDAC9, Hedgehog, HGF, HIFIA, HIP1, histamine and histamine receptors, HLA-A, HLA-DRA, HM74, HMOXI, HSP90, HUMCYT2A, ICEBERG, ICOSL, ID2, IFN-α, IFNA1, IFNA2, IFNA4, IFNA5, EFNA6, BFNA7, IFNB1, IFNgamma, IFNW1, IGBP1, IGF1, IGFIR, IGF2, IGFBP2, IGFBP3, IGFBP6, DL-1, ILIO, ILIORA, ILIORB, IL-1, IL1R1 (CD121a), IL1R2(CD121b), IL-IRA, IL-2, IL2RA (CD25), IL2RB(CD122), IL2RG(CD132), IL-4, IL-4R(CD123), IL-5, IL5RA(CD125), IL3RB(CD131), IL-6, IL6RA, (CD126), IR6RB(CD130), IL-7, IL7RA(CD127), IL-8, CXCR1 (IL8RA), CXCR2, (IL8RB/CD128), IL-9, IL9R (CD129), IL-10, IL10RA(CD210), IL10RB(CDW210B), IL-11, IL11 RA, IL-12, IL-12A, IL-12B, IL-12RB1, IL-12RB2, IL-13, IL13RA1, IL13RA2, IL14, IL15, IL15RA, 1L16, IL17, IL17A, IL17B, IL17C, IL17R, IL18, IL18BP, IL18R1, IL18RAP, IL19, ILIA, ILIB, IL1F10, IL1F5, IL1F6, IL1F7, IL1F8, DL1F9, ILIHYI, ILIR1, IL1R2, ILIRAP, ILIRAPLI, IL1RAPL2, IL1RL1, IL1RL2, ILIRN, IL2, IL20, IL20RA, IL21R, IL22, IL22R, IL22RA2, IL23, DL24, IL25, IL26, IL27, IL28A, IL28B, IL29, IL2RA, IL2RB, IL2RG, IL3, IL30, IL3RA, IL4, IL4R, IL6ST (glycoprotein 130), ILK, INHA, INHBA, INSL3, INSL4, IRAK1, IRAK2, ITGA1, ITGA2, ITGA3, ITGA6 (a6 integrin), ITGAV, ITGB3, ITGB4 (134 integrin), JAG1, JAK1, JAK3, JTB, JUN, K6HF, KAI1, KDR, KITLG, KLF5 (GC Box BP), KLF6, KLK1O, KLK12, KLK13, KLK14, KLK15, KLK3, KLK4, KLK5, KLK6, KLK9, KRT1, KRT19 (Keratin 19), KRT2A, KRTHB6 (hair-specific type II keratin), LAMAS, LEP (leptin), Lingo-p75, Lingo-Troy, LPS, LTA (TNF-b), LTB, LTB4R (GPR16), LTB4R2, LTBR, MACMARCKS, MAG or Omgp, MAP2K7 (c-Jun), MCP-1, MDK, MIB1, midkine, MIF, MISRII, MJP-2, MK, MKI67 (Ki-67), MMP2, MMP9, MS4A1, MSMB, MT3 (metallothionectin-UI), mTOR, MTSS1, MUC1 (mucin), MYC, MYD88, NCK2, neurocan, NFKBI, NFKB2, NGFB (NGF), NGFR, NgR-Lingo, NgRNogo66, (Nogo), NgR-p75, NgR-Troy, NMEI (NM23A), NOTCH, NOTCH1, NOX5, NPPB, NROB1, NROB2, NRID1, NR1D2, NR1H2, NR1H3, NR1H4, NR1I2, NR1I3, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, NRP1, NRP2, NT5E, NTN4, ODZI, OPRDI, P2RX7, PAP, PART1, PATE, PAWR, PCA3, PCDGF, PCNA, PDGFA, PDGFB, PDGFRA, PDGFRB, PECAMI, peg-asparaginase, PF4 (CXCL4), PGF, PGR, phosphacan, PIAS2, PI3 Kinase, PIK3CG, PLAU (uPA), PLG, PLXDCI, PKC, PKC-beta, PPBP (CXCL7), PPID, PRI, PRKCQ, PRKD1, PRL, PROC, PROK2, PSAP, PSCA, PTAFR, PTEN, PTGS2 (COX-2), PTN, RAC2 (P21Rac2), RANK, RANK ligand, RARB, RGS1, RGS13, RGS3, RNFI1O (ZNF144), Ron, ROBO2, RXR, S100A2, SCGB 1D2 (lipophilin B), SCGB2A1 (mammaglobin 2), SCGB2A2 (mammaglobin 1), SCYE1 (endothelial Monocyte-activating cytokine), SDF2, SERPENA1, SERPINA3, SERPINB5 (maspin), SERPINEI (PAI-I), SERPINFI, SHIP-1, SHIP-2, SHB1, SHB2, SHBG, SfcAZ, SLC2A2, SLC33A1, SLC43A1, SLIT2, SPP1, SPRR1B (Spr1), ST6GAL1, STAB1, STATE, STEAP, STEAP2, TB4R2, TBX21, TCP1O, TDGF1, TEK, TGFA, TGFB1, TGFB1I1, TGFB2, TGFB3, TGFBI, TGFBR1, TGFBR2, TGFBR3, THIL, THBS1 (thrombospondin-1), THBS2, THBS4, THPO, TIE (Tie-1), TIMP3, tissue factor, TLR1O, TLR2, TLR3, TLR4, TLR5, TLR6, TLR7, TLR8, TLR9, TNF, TNF-a, TNFAIP2 (B94), TNFAIP3, TNFRSFI1A, TNFRSF1A, TNFRSF1B, TNFRSF21, TNFRSF5, TNFRSF6 (Fas), TNFRSF7, TNFRSF8, TNFRSF9, TNFSF1O (TRAIL), TNFSF1 1 (TRANCE), TNFSF12 (APO3L), TNFSF13 (April), TNFSF13B, TNFSF14 (HVEM-L), TNFSF15 (VEGI), TNFSFI8, TNFSF4 (OX40 ligand), TNFSF5 (CD40 ligand), TNFSF6 (FasL), TNFSF7 (CD27 ligand), TNFSF8 (CD30 ligand), TNFSF9 (4-1BB ligand), TOLLIP, Toll-like receptors, TOP2A (topoisomerase lia), TP53, TPM1, TPM2, TRADD, TRAF1, TRAF2, TRAF3, TRAF4, TRAF5, TRAF6, TRKA, TREM1, TREM2, TRPC6, TSLP, TWEAK, Tyrosinase, uPAR, VEGF, VEGFB, VEGFC, versican, VHL C5, VLA-4, Wnt-1, XCL1 (Iymphotactin), XCL2 (SCM-Ib), XCRI (GPR5/CCXCR1), YY1, and ZFPM2.

In some embodiments, antibodies lacking interchain cysteines herein comprise an epitope binding domain selected from the group consisting of: abagovomab, abatacept (also know as ORENCIA®), abciximab (also known as REOPRO®, c7E3 Fab), adalimumab (also known as HUMIRA®), adecatumumab, alemtuzumab (also known as CAMPATH®, MabCampath or Campath-1H), altumomab, afelimomab, anatumomab mafenatox, anetumumab, anrukizumab, apolizumab, arcitumomab, aselizumab, atlizumab, atorolimumab, bapineuzumab, basiliximab (also known as SIMULECT®), bavituximab, bectumomab (also known as LYMPHOSCAN®), belimumab (also known as LYMPHO-STAT-B®), bertilimumab, besilesomab, bevacizumab (also known as AVASTIN®), biciromab brallobarbital, bivatuzumab mertansine, campath, canakinumab (also known as ACZ885), cantuzumab mertansine, capromab (also known as PROSTASCINT®), catumaxomab (also known as REMOVAB®), cedelizumab (also known as CIMZIA®), certolizumab pegol, cetuximab (also known as ERBITUX®), clenoliximab, dacetuzumab, dacliximab, daclizumab (also known as ZENAPAX®), denosumab (also known as AMG 162), detumomab, dorlimomab aritox, dorlixizumab, duntumumab, durimulumab, durmulumab, ecromeximab, eculizumab (also known as SOLIRIS®), edobacomab, edrecolomab (also known as Mab17-1A, PANOREX®), efalizumab (also known as RAPTIVA®), efungumab (also known as MYCOGRAB®), elsilimomab, enlimomab pegol, epitumomab cituxetan, efalizumab, epitumomab, epratuzumab, erlizumab, ertumaxomab (also known as REXOMUN®), etanercept (also known as ENBREL®), etaracizumab (also known as etaratuzumab, VITAXIN®, ABEGRIN™), exbivirumab, fanolesomab (also known as NEUTROSPEC®), faralimomab, felvizumab, fontolizumab (also known as HUZAF®), galiximab, gantenerumab, gavilimomab (also known as ABXCBL®), gemtuzumab ozogamicin (also known as MYLOTARG®), golimumab (also known as ONTO 148), gomiliximab, ibalizumab (also known as TNX-355), ibritumomab tiuxetan (also known as ZEVALIN®), igovomab, imciromab, infliximab (also known as REMICADE®), inolimomab, inotuzumab ozogamicin, ipilimumab (also known as MDX-010, MDX-101), iratumumab, keliximab, labetuzumab, lemalesomab, lebrilizumab, lerdelimumab, lexatumumab (also known as, HGS-ETR2, ETR2-ST01), lexitumumab, libivirumab, lintuzumab, lucatumumab, lumiliximab, mapatumumab (also known as HGSETR1, TRM-1), maslimomab, matuzumab (also known as EMD72000), mepolizumab (also known as BOSATRIA®), metelimumab, milatuzumab, minretumomab, mitumomab, morolimumab, motavizumab (also known as NUMAX™), muromonab (also known as OKT3), nacolomab tafenatox, naptumomab estafenatox, natalizumab (also known as TYSABRI®, ANTEGREN®), nebacumab, nerelimomab, nimotuzumab (also known as THERACIM hR3®, THERA-CIM-hR3®, THERALOC®), nofetumomab merpentan (also known as VERLUMA®), ocrelizumab, odulimomab, ofatumumab, omalizumab (also known as XOLAIR®), oregovomab (also known as OVAREX®), otelixizumab, pagibaximab, palivizumab (also known as SYNAGIS®), panitumumab (also known as ABX-EGF, VECTIBIX®), pascolizumab, pemtumomab (also known as THERAGYN®), pertuzumab (also known as 2C4, OMNITARG®), pexelizumab, pintumomab, priliximab, pritumumab, ranibizumab (also known as LUCENTIS®), raxibacumab, regavirumab, reslizumab, rituximab (also known as RITUXAN®, MabTHERA®), rovelizumab, ruplizumab, satumomab, sevirumab, sibrotuzumab, siplizumab (also known as MEDI-507), sontuzumab, stamulumab (also known as MYO-029), sulesomab (also known as LEUKOSCAN®), tacatuzumab tetraxetan, tadocizumab, talizumab, taplitumomab paptox, tefibazumab (also known as AUREXIS®), telimomab aritox, teneliximab, teplizumab, ticilimumab, tocilizumab (also known as ACTEMRA®), toralizumab, tositumomab, trastuzumab (also known as HERCEPTIN®), tremelimumab (also known as CP-675,206), tucotuzumab celmoleukin, tuvirumab, urtoxazumab, ustekinumab (also known as ONTO 1275), vapaliximab, veltuzumab, vepalimomab, visilizumab (also known as NUVION®), volociximab (also known as M200), votumumab (also known as HUMASPECT®), zalutumumab, zanolimumab (also known as HuMAX-CD4), ziralimumab, or zolimomab aritox.

In certain embodiments, antibodies lacking interchain cysteines herein comprise an epitope binding domain that binds to the same antigen as the antibodies listed above. In some embodiments, antibodies herein comprise an epitope binding domain that competes for binding with an antibody selected from selected from the antibodies listed above.

In vitro binding of an antibody herein may be quantified by means known in the art, for example microarrays, immobilization on a BIAcore® chip, enzyme linked immunoabsorbant assay (ELISA). In certain embodiments, chromogenic reporters and substrates produce an observable color change to indicate the presence of antigen or analyte. In some embodiments assay techniques utilize fluorogenic, electrochemiluminescent, real-time PCR reporters, and electroimmunoassays to create quantifiable signals.

In vivo binding of an antibody herein may be assayed in biological samples using in situ determination. A biological sample may be any tissue, substance or fluid as described below. In situ binding determination techniques include without limitation, staining, dyes, fluorescent labels, radiographic assays, tapping mode atomic force microscopy, and micro-radioisotopic antiglobulin assay.

The modifications inherent in cysteine engineering may affect binding specificity. For example, in some embodiments an antibody lacking interchain cysteines herein does not bind to a human leucocyte receptor. In certain embodiments an antibody herein does not bind to a FcγRIII receptor. The engineered antibody, however, closely mimics the binding of native antibody to the protein kinase domain of certain cell surface receptor molecules including human neonatal Fc leucocyte receptor (FcLR), epidermal growth factor receptor (EGFR) and HER3. Fluorescence indicates binding of an anti-EGFR antibody lacking interchain cysteines EGFR expressed on a cell surface. Saporin-conjugated anti-Her3 (mAb) and anti-Her3 (antibody lacking interchain cysteines) show similar killing potency on SKBr3 cells, suggesting that the mAb and antibody lacking interchain cysteines have similar binding and internalization kinetics.

### Expression Systems

Recombinant expression of an antibody herein, derivative, analog or fragment thereof, requires construction of an expression vector containing a polynucleotide that encodes the antibody. Once a polynucleotide encoding an antibody or a heavy or light chain of an antibody, or portion thereof, herein has been obtained, the vector for the production of the antibody may be produced by recombinant DNA technology using techniques known in the art. Thus, methods for preparing a protein by expressing a polynucleotide containing an antibody encoding nucleotide sequence are described herein. Methods which are known in the art can be used to construct expression vectors containing antibody coding sequences and appropriate transcriptional and translational control signals. These methods include, for example, in vitro recombinant DNA techniques, synthetic techniques, and in vivo genetic recombination.

Herein provided, thus, are replicable vectors comprising a nucleotide sequence encoding an antibody herein, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody and the variable domain of the antibody may be cloned into such a vector for expression of the entire heavy chain, the entire light chain, or both the entire heavy and light chains.

The expression vector is transferred to a host cell by conventional techniques and the transfected cells are then cultured by conventional techniques to produce an antibody herein. Thus, provided herein are host cells containing a polynucleotide encoding an antibody herein or fragments thereof, or a heavy or light chain thereof, or portion thereof, or a single chain antibody herein, operably linked to a heterologous promoter. In certain embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibodies herein. Such host-expression systems represent vehicles by which the coding sequences of interest may be produced and subsequently purified, but also represent cells which may, when transformed or transfected with the appropriate nucleotide coding sequences, express an antibody herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces; Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

Bacterial cells such as Escherichia coli, and eukaryotic cells, may be used for the expression of a recombinant antibody. For non-limiting example, mammalian cells such as Chinese hamster ovary cells (CHO), in conjunction with a vector such as the major intermediate early gene promoter element from human cytomegalovirus can be an effective expression system for antibodies.

In bacterial systems, a number of expression vectors may be advantageously selected depending upon the use intended for the antibody being expressed. For example, when a large quantity of such a protein is to be produced, for the generation of pharmaceutical compositions of an antibody, vectors which direct the expression of high levels of fusion protein products that are readily purified may be desirable. Such vectors include, but are not limited to, the E. coli expression vector pUR278, in which the antibody coding sequence may be ligated individually into the vector in frame with the lac Z coding region so that a fusion protein is produced; pIN vectors and the like. PGEX vectors may also be used to express foreign polypeptides as fusion proteins with glutathione 5-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis In an insect system, Autographa californica nuclear polyhedrosis virus (AcNPV) is used as a vector to express foreign genes. The virus grows in Spodoptera frugiperda cells. The antibody coding sequence may be cloned individually into nonessential regions of the virus and placed under control of an AcNPV promoter.

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a nonessential region of the viral genome (e.g., region EI or E3) may result in a recombinant virus that is viable and capable of expressing the antibody in infected hosts. Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc.

In some embodiments, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138, BT483, Hs578T, HTB2, BT2O, NS1 and T47D, NS0 (a murine myeloma cell line that does not endogenously produce any immunoglobulin chains), CRL7O3O and HsS78Bst cells.

For long-term, high-yield production of recombinant proteins, stable expression is appropriate. For example, cell lines which stably express the antibody may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may be used to engineer cell lines which express the antibody. Such engineered cell lines may be useful in screening and evaluation of compositions that interact directly or indirectly with the antibody.

In certain embodiments, antibodies presented herein are expressed in a cell line with transient expression of the antibody. Transient transfection is a process in which the nucleic acid introduced into a cell does not integrate into the genome or chromosomal DNA of that cell but is maintained as an extrachromosomal element, e.g. as an episome, in the cell. Transcription processes of the nucleic acid of the episome are not affected and a protein encoded by the nucleic acid of the episome is produced.

The cell line, either stable or transiently transfected, is maintained in cell culture medium and conditions known in the art resulting in the expression and production of monoclonal antibodies. In certain embodiments, the mammalian cell culture media is based on commercially available media formulations, including, for example, DMEM or Ham's F12. In some embodiments, the cell culture media is modified to support increases in both cell growth and biologic protein expression. As used herein, the terms “cell culture medium,” “culture medium,” and “medium formulation” refer to a nutritive solution for the maintenance, growth, propagation, or expansion of cells in an artificial in vitro environment outside of a multicellular organism or tissue. Cell culture medium may be optimized for a specific cell culture use, including, for example, cell culture growth medium which is formulated to promote cellular growth, or cell culture production medium which is formulated to promote recombinant protein production. The terms nutrient, ingredient, and component may be used interchangeably to refer to the constituents that make up a cell culture medium.

In various embodiments, the cell lines are maintained using a fed batch method. As used herein, “fed batch method,” refers to a method by which a fed batch cell culture is supplied with additional nutrients after first being incubated with a basal medium. For example, a fed batch method may comprise adding supplemental media according to a determined feeding schedule within a given time period. Thus, a “fed batch cell culture” refers to a cell culture where the cells, typically mammalian, and culture medium are supplied to the culturing vessel initially and additional culture nutrients are fed, continuously or in discrete increments, to the culture during culturing, with or without periodic cell and/or product harvest before termination of culture.

The cell culture medium used and the nutrients contained therein are known to one of skill in the art. In some embodiments, the cell culture medium comprises a basal medium and at least one hydrolysate, e.g., soy-based, hydrolysate, a yeast-based hydrolysate, or a combination of the two types of hydrolysates resulting in a modified basal medium. The additional nutrients may sometimes include only a basal medium, such as a concentrated basal medium, or may include only hydrolysates, or concentrated hydrolysates. Suitable basal media include, but are not limited to Dulbecco's Modified Eagle's Medium (DMEM), DME/F12, Minimal Essential Medium (MEM), Basal Medium Eagle (BME), RPMI 1640, F-10, F-12, α-Minimal Essential Medium (α-MEM), Glasgow's Minimal Essential Medium (G-MEM), PF CHO (see, e.g., CHO protein free medium (Sigma) or EX-CELL™ 325 PF CHO Serum-Free Medium for CHO Cells Protein-Free (SAFC Bioscience), and Iscove's Modified Dulbecco's Medium. Other examples of basal media which may be used in the technology herein include BME Basal Medium (Gibco-Invitrogen; Dulbecco's Modified Eagle Medium (DMEM, powder) (Gibco-Invitrogen (#31600)).

In certain embodiments, the basal medium may be is serum-free, meaning that the medium contains no serum (e.g., fetal bovine serum (FBS), horse serum, goat serum, or any other animal-derived serum known to one skilled in the art) or animal protein free media or chemically defined media.

The basal medium may be modified in order to remove certain non-nutritional components found in standard basal medium, such as various inorganic and organic buffers, surfactant(s), and sodium chloride. Removing such components from basal cell medium allows an increased concentration of the remaining nutritional components, and may improve overall cell growth and protein expression. In addition, omitted components may be added back into the cell culture medium containing the modified basal cell medium according to the requirements of the cell culture conditions. In certain embodiments, the cell culture medium contains a modified basal cell medium, and at least one of the following nutrients, an iron source, a recombinant growth factor; a buffer; a surfactant; an osmolarity regulator; an energy source; and non-animal hydrolysates. In addition, the modified basal cell medium may optionally contain amino acids, vitamins, or a combination of both amino acids and vitamins. In some embodiments, the modified basal medium further contains glutamine, e.g, L-glutamine, and/or methotrexate.

In some embodiments, antibody production is conducted in large quantity by a bioreactor process using fed-batch, batch, perfusion or continuous feed bioreactor methods known in the art. Large-scale bioreactors have at least 1000 liters of capacity, sometimes about 1,000 to 100,000 liters of capacity. These bioreactors may use agitator impellers to distribute oxygen and nutrients. Small scale bioreactors refers generally to cell culturing in no more than approximately 100 liters in volumetric capacity, and can range from about 1 liter to about 100 liters. Alternatively, single-use bioreactors (SUB) may be used for either large-scale or small scale culturing.

Temperature, pH, agitation, aeration and inoculum density may vary depending upon the host cells used and the recombinant protein to be expressed. For example, a recombinant protein cell culture may be maintained at a temperature between 30 and 45 degrees Celsius. The pH of the culture medium may be monitored during the culture process such that the pH stays at an optimum level, which may be for certain host cells, within a pH range of 6.0 to 8.0. An impellor driven mixing may be used for such culture methods for agitation. The rotational speed of the impellor may be approximately 50 to 200 cm/sec tip speed, but other airlift or other mixing/aeration systems known in the art may be used, depending on the type of host cell being cultured. Sufficient aeration is provided to maintain a dissolved oxygen concentration of approximately 20% to 80% air saturation in the culture, again, depending upon the selected host cell being cultured. Alternatively, a bioreactor may sparge air or oxygen directly into the culture medium. Other methods of oxygen supply exist, including bubble-free aeration systems employing hollow fiber membrane aerators. A number of selection systems may be used, including but not limited to, the herpes simplex virus thymidine kinase, glutamine synthetase, hypoxanthine guanine phosphoribosyltransferase, and adenine phosphoribosyltransferase genes can be employed in tk-, gs-, hgprt- or aprt-cells, respectively. Also, antimetabolite resistance can be used as the basis of selection for the following genes: dhfr, which confers resistance to methotrexate gpt, which confers resistance to mycophenolic acid, neo, which confers resistance to the aminoglycoside G-418, and hygro, which confers resistance to hygromycin.

Methods known in the art of recombinant DNA technology may be applied to select the desired recombinant clone. including but not limited to, the herpes simplex virus thymidine kinase, glutamine synthetase, hypoxanthine guanine phosphoribosyltransferase, and adenine phosphoribosyltransferase genes can be employed in tk-, gs-, hgprt- or aprt-cells, respectively. Also, antimetabolite resistance can be used as the basis of selection for the following genes: dhfr, which confers resistance to methotrexate; gpt, which confers resistance to mycophenolic acid; neo, which confers resistance to the aminoglycoside G-418; and hygro, which confers resistance to hygromycin. Methods known in the art of recombinant DNA technology may be applied to select the desired recombinant clone.

The expression levels of an antibody can be increased by vector amplification. When a marker in the vector system expressing antibody is amplifiable, increase in the level of inhibitor present in culture of host cell may increase the number of copies of the marker gene. Since the amplified region is associated with the antibody gene, production of the antibody may also increase.

The host cell may be co-transfected with two expression vectors of the antibodies herein; the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain. The coding sequences for the heavy and light chains may comprise cDNA or genomic DNA.

Once an antibody lacking interchain cysteines has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies herein or fragments thereof may be fused to heterologous polypeptide sequences described herein or otherwise known in the art to facilitate purification.

### Phage Display Techniques

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage.

Phage used in these methods are typically filamentous phage including fd and M13 and the VH and VL domains are usually recombinantly fused to either the phage gene III or gene VIII. Phage expressing an antigen binding domain that binds to an epitope of interest can be selected or identified with antigen, e.g., using labeled antigen or antigen bound or captured to a solid surface or bead. Phage display methods are known in the art.

After phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′) 2 fragments can also be employed using methods known in the art.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. The vectors for expressing the VH or VL domains sometimes comprise an EF-1α promoter, a secretion signal, a cloning site for the variable domain, constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known in the art.

### Nucleic Acids

A polynucleotide may be obtained, and the nucleotide sequence of the polynucleotide determined, by any method known in the art. Since the amino acid sequences of antibodies are known, nucleotide sequences encoding these antibodies can be determined using methods known in the art, e.g., nucleotide codons known to encode particular amino acids are assembled in such a way to generate a nucleic acid that encodes the antibody or fragment thereof herein. Such a polynucleotide encoding the antibody may be assembled from chemically synthesized oligonucleotides which, briefly, involves the synthesis of overlapping oligonucleotides containing portions of the sequence encoding the antibody, annealing and ligating of those oligonucleotides, and then amplification of the ligated oligonucleotides by PCR.

In some embodiments, a polynucleotide encoding an antibody may be generated from nucleic acid from a suitable source. If a clone containing a nucleic acid encoding a particular antibody is not available, but the sequence of the antibody is known, a nucleic acid encoding the immunoglobulin may be chemically synthesized or obtained from a suitable source (e.g., an antibody cDNA library, or a cDNA library generated from, or nucleic acid, sometimes poly A+RNA, isolated from, any tissue or cells expressing the antibody by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method known in the art.

Once the nucleotide sequence of the antibody is determined, the nucleotide sequence of the antibody may be manipulated using methods known in the art for the manipulation of nucleotide sequences, e.g., recombinant DNA techniques, site directed mutagenesis, PCR, etc. to generate antibodies having a different amino acid sequence, for example to create amino acid substitutions, deletions, and/or insertions.

In certain embodiments, one or more of the CDRs is inserted within framework regions using recombinant DNA techniques. The framework regions may be naturally occurring or consensus framework regions, and sometimes human framework regions. The polynucleotide generated by the combination of the framework regions and CDRs may encode an antibody that specifically binds to EGFR, HER3, or other selected antigen. In some embodiments, as discussed supra, one or more amino acid substitutions may be made within the framework regions, and the amino acid substitutions may improve binding of the antibody to its antigen. Additionally, such methods may be used to make amino acid substitutions or deletions of one or more variable region cysteine residues participating in an intrachain disulfide bond to generate antibodies lacking one or more intrachain disulfide bonds. Other alterations to the polynucleotide are encompassed by the present disclosure and within the skill of the art.

Nucleic acid sequences for exemplarly antibodies of the disclosure are provided below. The codons that are altered are indicated in bold and underline text.

>mAb-VL-CL-kappa-anti-EGFR
(SEQ ID NO: 15)
GACATCCAGATGACCCAGAGCCCCAGCAGCCTGAGCGCCAGCGTGGGCGA
CAGAGTGACCATCACCTGCCAGGCCAGCCAGGACATCAGCAACTACCTGA
ACTGGTATCAGCAGAAGCCCGGCAAGGCCCCCAAGCTGCTGATCTACGAC
GCCAGCAACCTGGAGACAGGCGTGCCCAGCAGATTCAGCGGCAGCGGCTC
CGGCACCGACTTCACCTTCACCATCAGCAGCCTCCAGCCCGAGGATATCG
CCACCTACTTTTGCCAGCACTTCGACCACCTGCCCCTGGCCTTTGGCGGC
GGAACAAAGGTGGAGATCAAGCGTACGGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGT
>mAb-VH-CH1gamma1-CH2gamma2-CH3gamma3-anti-EGFR
(SEQ ID NO: 16)
CAGGTGCAGCTCCAGGAGAGCGGCCCTGGCCTGGTGAAGCCCAGCGAGAC
ACTGAGCCTGACCTGCACCGTGTCCGGCGGCAGCGTGTCCAGCGGCGACT
ACTACTGGACCTGGATCAGACAGAGCCCCGGCAAGGGCCTGGAGTGGATC
GGCCACATCTACTACAGCGGCAACACCAACTACAACCCCAGCCTGAAGTC
CAGACTGACCATCAGCATCGACACCAGCAAGACCCAGTTCAGCCTGAAGC
TGTCCAGCGTGACAGCCGCCGACACCGCCATCTACTACTGCGTGAGAGAC
AGAGTGACCGGCGCTTTCGACATCTGGGGCCAGGGCACCATGGTGACCGT
GTCCAGCGCGTCGACCAAGGGCCCATCcGTCTTCCCCCTGGCACCCTCCT
CCAAGAGCACCTCTGGGGGCACAGCGGCCCTGGGCTGCCTGGTCAAGGAC
TACTTCCCCGAACCGGTGACGGTGTCCTGGAACTCAGGCGCtCTGACCAG
CGGCGTGCACACCTTCCCGGCTGTCCTACAGTCCTCAGGACTCTACTCCC
TCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGT
TGAGCCCAAATCTTGTGACAAAACTCACACATGCCCACCGTGCCCAGCAC
CTGAACTCCTGGGGGGACCGTCAGTCTTCCTCTTCCCCCCAAAACCCAAG
GACACCCTCATGATCTCCCGGACCCCTGAGGTCACATGCGTGGTGGTGGA
CGTGAGCCACGAAGACCCTGAGGTCAAGTTCAACTGGTACGTGGACGGCG
TGGAGGTGCATAATGCCAAGACAAAGCCGCGGGAGGAGCAGTACAACAGC
ACGTACCGTGTGGTCAGCGTCCTCACCGTCCTGCACCAGGACTGGCTGAA
TGGCAAGGAGTACAAGTGCAAGGTCTCCAACAAAGCCCTCCCAGCCCCCA
TCGAGAAAACCATCTCCAAAGCCAAAGGGCAGCCCCGAGAACCACAGGTc
TACACCCTGCCCCCATCCCGGGAGGAGATGACCAAGAACCAGGTCAGCCT
GACCTGCCTGGTCAAAGGCTTCTATCCCAGCGACATCGCCGTGGAGTGGG
AGAGCAATGGGCAGCCGGAGAACAACTACAAGACCACGCCTCCCGTGCTG
GACTCCGACGGCTCCTTCTTCCTCTATAGCAAGCTCACCGTGGACAAGAG
CAGGTGGCAGCAGGGGAACGTCTTCTCATGCTCCGTGATGCATGAGGCTC
TGCACAACCACTACACGCAGAAGAGCTTAAGCCTGTCTCCGGGTAAA
>mAb-Val-VL-CL-kappa-anti-EGFR
(SEQ ID NO: 17)
GACATCCAGATGACCCAGAGCCCCAGCAGCCTGAGCGCCAGCGTGGGCGA
CAGAGTGACCATCACCTGCCAGGCCAGCCAGGACATCAGCAACTACCTGA
ACTGGTATCAGCAGAAGCCCGGCAAGGCCCCCAAGCTGCTGATCTACGAC
GCCAGCAACCTGGAGACAGGCGTGCCCAGCAGATTCAGCGGCAGCGGCTC
CGGCACCGACTTCACCTTCACCATCAGCAGCCTCCAGCCCGAGGATATCG
CCACCTACTTTTGCCAGCACTTCGACCACCTGCCCCTGGCCTTTGGCGGC
GGAACAAAGGTGGAGATCAAGCGTACGGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGGTC
>mAb-Val-VH-CH1gamma1-CH2gamma2-CH3gamma3-anti-
EGFR
(SEQ ID NO: 18)
CAGGTGCAGCTCCAGGAGAGCGGCCCTGGCCTGGTGAAGCCCAGCGAGAC
ACTGAGCCTGACCTGCACCGTGTCCGGCGGCAGCGTGTCCAGCGGCGACT
ACTACTGGACCTGGATCAGACAGAGCCCCGGCAAGGGCCTGGAGTGGATC
GGCCACATCTACTACAGCGGCAACACCAACTACAACCCCAGCCTGAAGTC
CAGACTGACCATCAGCATCGACACCAGCAAGACCCAGTTCAGCCTGAAGC
TGTCCAGCGTGACAGCCGCCGACACCGCCATCTACTACTGCGTGAGAGAC
AGAGTGACCGGCGCTTTCGACATCTGGGGCCAGGGCACCATGGTGACCGT
GTCCAGCGCGTCGACCAAGGGCCCATCcGTCTTCCCCCTGGCACCCTCCT
CCAAGAGCACCTCTGGGGGCACAGCGGCCCTGGGCTGCCTGGTCAAGGAC
TACTTCCCCGAACCGGTGACGGTGTCCTGGAACTCAGGCGCtCTGACCAG
CGGCGTGCACACCTTCCCGGCTGTCCTACAGTCCTCAGGACTCTACTCCC
TCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGT
TGAGCCCAAATCGTCTGACAAAACTCACACAGTCCCACCGGTCCCAGCAC
CTGAACTCCTGGGGGGACCGTCAGTCTTCCTCTTCCCCCCAAAACCCAAG
GACACCCTCATGATCTCCCGGACCCCTGAGGTCACATGCGTGGTGGTGGA
CGTGAGCCACGAAGACCCTGAGGTCAAGTTCAACTGGTACGTGGACGGCG
TGGAGGTGCATAATGCCAAGACAAAGCCGCGGGAGGAGCAGTACAACAGC
ACGTACCGTGTGGTCAGCGTCCTCACCGTCCTGCACCAGGACTGGCTGAA
TGGCAAGGAGTACAAGTGCAAGGTCTCCAACAAAGCCCTCCCAGCCCCCA
TCGAGAAAACCATCTCCAAAGCCAAAGGGCAGCCCCGAGAACCACAGGTc
TACACCCTGCCCCCATCCCGGGAGGAGATGACCAAGAACCAGGTCAGCCT
GACCTGCCTGGTCAAAGGCTTCTATCCCAGCGACATCGCCGTGGAGTGGG
AGAGCAATGGGCAGCCGGAGAACAACTACAAGACCACGCCTCCCGTGCTG
GACTCCGACGGCTCCTTCTTCCTCTATAGCAAGCTCACCGTGGACAAGAG
CAGGTGGCAGCAGGGGAACGTCTTCTCATGCTCCGTGATGCATGAGGCTC
TGCACAACCACTACACGCAGAAGAGCTTAAGCCTGTCTCCGGGTAAA

### Scalable Production of Antibodies Lacking Interchain Cysteines

An antibody lacking interchain cysteines may be produced by a scalable process. In some embodiments, antibodies lacking interchain cysteines may be produced by a scalable process in the research laboratory that may be scaled up to produce the proteins in analytical scale bioreactors (for example, but not limited to 5 L, 10 L, 15 L, 30 L, or 50 L bioreactors) while maintaining the functional activity of the proteins. For instance, in an embodiment, proteins produced by scalable processes exhibit low to undetectable levels of aggregation as measured by HPSEC or rCGE, that is, no more than 5%, no more than 4%, no more than 3%, no more than 2%, no more than 1%, or no more than 0.5% aggregate by weight protein, and/or low to undetectable levels of fragmentation, that is, 80% or higher, 85% or higher, 90% or higher, 95% or higher, 98% or higher, or 99% or higher, or 99.5% or higher of the total peak area in the peak(s) representing intact antibodies lacking interchain cysteines.

In certain embodiments, the antibodies lacking interchain cysteines may be produced by a scalable process in the research laboratory that may be scaled up to produce the proteins in production scale bioreactors (for example, but not limited to 75 L, 100 L, 150 L, 300 L, or 500 L). In some embodiments, the scalable process results in little or no reduction in production efficiency as compared to the production process performed in the research laboratory. In some embodiments, the scalable process produces antibodies lacking interchain cysteines at production efficiency of about 10 mg/L, about 20 m/L, about 30 mg/L, about 50 mg/L, about 75 mg/L, about 100 mg/L, about 125 mg/L, about 150 mg/L, about 175 mg/L, about 200 mg/L, about 250 mg/L, about 300 mg/L or higher.

In various embodiments, the scalable process produces antibodies lacking interchain cysteines at production efficiency of at least about 10 mg/L, at least about 20 mg/L, at least about 30 mg/L, at least about 50 mg/L, at least about 75 mg/L, at least about 100 mg/L, at least about 125 mg/L, at least about 150 mg/L, at least about 175 mg/L, at least about 200 mg/L, at least about 250 mg/L, at least about 300 mg/L or higher.

In some embodiments, the scalable process produces antibodies lacking interchain cysteines at production efficiency from about 10 mg/L to about 300 mg/L, from about 10 mg/L to about 250 mg/L, from about 10 mg/L to about 200 mg/L, from about 10 mg/L to about 175 mg/L, from about 10 mg/L to about 150 mg/L, from about 10 mg/L to about 100 mg/L, from about 20 mg/L to about 300 mg/L, from about 20 mg/L to about 250 mg/L, from about 20 mg/L to about 200 mg/L, from 20 mg/L to about 175 mg/L, from about 20 mg/L to about 150 mg/L, from about 20 mg/L to about 125 mg/L, from about 20 mg/L to about 100 mg/L, from about 30 mg/L to about 300 mg/L, from about 30 mg/L to about 250 mg/L, from about 30 mg/L to about 200 mg/L, from about 30 mg/L to about 175 mg/L, from about 30 mg/L to about 150 mg/L, from about 30 mg/L to about 125 mg/L, from about 30 mg/L to about 100 mg/L, from about 50 mg/L to about 300 mg/L, from about 50 mg/L to about 250 mg/L, from about 50 mg/L to about 200 mg/L, from 50 mg/L to about 175 mg/L, from about 50 mg/L to about 150 mg/L, from about 50 mg/L to about 125 mg/L, from about 50 mg/L to about 100 mg/L.

### Antibody Purification and Isolation

Once an antibody molecule has been produced by recombinant or hybridoma expression, it may be purified by any method known in the art for purification of an immunoglobulin molecule, for example, by chromatography (e.g., ion exchange, affinity, particularly by affinity for the specific antigens Protein A or Protein G, and sizing column chromatography), centrifugation, differential solubility, or by any other standard technique for the purification of proteins. Further, the antibodies of the present technology or fragments thereof may be fused to heterologous polypeptide sequences (referred to herein as “tags”) described above or otherwise known in the art to facilitate purification.

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. For example, procedures for isolating antibodies which are secreted into the periplasmic space of E. coli are known in the art. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, hydrophobic interaction chromatography, ion exchange chromatography, gel electrophoresis, dialysis, and/or affinity chromatography either alone or in combination with other purification steps. The suitability of protein A as an affinity ligand depends on the species and isotype of any immunoglobulin Fc domain that is present in the antibody and will be understood by one of skill in the art. The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin, SEPHAROSE chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, and performed at low salt concentrations (e.g., from about 0-0.25 M salt).

Thus, in certain embodiments, antibodies as provided herein are substantially purified/isolated. In an embodiment, these isolated/purified recombinantly expressed antibodies may be administered to a patient to mediate a prophylactic or therapeutic effect. In some embodiments these isolated/purified antibodies may be used to diagnose a disease.

### Stability

Suitable stability assays are available in the art. In an embodiment, assays pertaining to the stability of proteins known in the art are applied to antibodies described herein to determine their stability. In certain embodiments, the stability of antibodies described herein can be assessed by aggregation and/or fragmentation rate or profile. To determine the level of aggregation or fragmentation, many techniques may be used. In some embodiments, the aggregation and/or fragmentation profile may be assessed by the use of analytical ultracentrifugation (AUC), size-exclusion chromatography (SEC), high performance size-exclusion chromatography (HPSEC), melting temperature (Tm), polyacrylamide gel electrophoresis (PAGE), capillary gel electrophoresis (CGE), light scattering (SLS), Fourier Transform Infrared Spectroscopy (FTIR), circular dichroism (CD), urea-induced protein unfolding techniques, intrinsic tryptophan fluorescence, differential scanning calorimetry, or 1-anilino-8-naphthalenesulfonic acid (ANS) protein binding techniques. In another embodiment, the stability of proteins herein is characterized by polyacrylamide gel electrophoresis (PAGE) analysis. In another embodiment, the stability of the proteins herein is characterized by size exclusion chromatography (SEC) profile analysis.

The thermal melting temperatures (Tm) of the Fab domain of an antibody can be a good indicator of the thermal stability of an antibody and may further provide an indication of the antibody shelf-life. A lower Tm indicates more aggregation and less stability, whereas a higher Tm indicates less aggregation and more stability. Thus, in certain embodiments antibodies having higher Tm are selected and utilized. Tm of a protein or protein fragment (e.g., a Fab domain) can be measured using any standard method known in the art, for example, by differential scanning calorimetry. Stability may be determined in vitro or in vivo. Stability may also be determined in an animal.

Antibodies, like all polypeptides, have an isoelectric point (pl), which is generally defined as the pH at which a polypeptide carries no net charge. Protein solubility is typically lowest when the pH of the solution is equal to the isoelectric point (pl) of the protein. As used herein the pl value is defined as the pl of the predominant charge form. The pl of a protein may be determined by a variety of methods including, but not limited to, isoelectric focusing and various computer algorithms.

The formation of at least one non-naturally occurring disulfide bond may influence the stability of an antibody lacking interchain cysteines herein in comparison to the antibody prior to modification. In some embodiments, the non-naturally occurring disulfide bond may increase stability of the antibody lacking interchain cysteines as compared to the same antibody prior to cysteine engineering. In various embodiments, the non-naturally occurring disulfide bond may decrease stability of an antibody lacking interchain cysteines as compared to the same antibody prior to cysteine engineering. In certain embodiments an antibody herein has a stability of about 70% or more compared to an antibody counterpart containing all native interchain cysteines. In some embodiments the stability is in vitro stability. In certain embodiments an antibody lacking interchain cysteines herein may be monomeric. In some embodiments an antibody herein may be dimeric.

In certain embodiments, an antibody lacking interchain cysteines has certain biochemical characteristics such as a particular isoelectric point (pl) or melting temperature (Tm). In some embodiments, an antibody lacking interchain cysteines has a pl ranging from 5.5 to 9.5. In certain embodiments, an antibody lacking interchain cysteines has a pl that ranges from about 5.5 to about 6.0, or about 6.0 to about 6.5, or about 6.5 to about 7.0, or about 7.0 to about 7.5, or about 7.5 to about 8.0, or about 8.0 to about 8.5, or about 8.5 to about 9.0, or about 9.0 to about 9.5. In various embodiments, an antibody lacking interchain cysteines has a pl that ranges from 5.5-6.0, or 6.0 to 6.5, or 6.5 to 7.0, or 7.0-7.5, or 7.5-8.0, or 8.0-8.5, or 8.5-9.0, or 9.0-9.5. An antibody lacking interchain cysteines sometimes has a pl of at least 5.5, or at least 6.0, or at least 6.3, or at least 6.5, or at least 6.7, or at least 6.9, or at least 7.1, or at least 7.3, or at least 7.5, or at least 7.7, or at least 7.9, or at least 8.1, or at least 8.3, or at least 8.5, or at least 8.7, or at least 8.9, or at least 9.1, or at least 9.3, or at least 9.5. In some embodiments, an antibody lacking interchain cysteines has a pl of at least about 5.5, or at least about 6.0, or at least about 6.3, or at least about 6.5, or at least about 6.7, or at least about 6.9, or at least about 7.1, or at least about 7.3, or at least about 7.5, or at least about 7.7, or at least about 7.9, or at least about 8.1, or at least about 8.3, or at least about 8.5, or at least about 8.7, or at least about 8.9, or at least about 9.1, or at least about 9.3, or at least about 9.5.

It is possible to optimize solubility by altering the number and location of ionizable residues in the antibody to adjust the pl. For example the pl of a polypeptide can be manipulated by making appropriate amino acid substitutions (e.g., by substituting a charged amino acid such as a lysine, for an uncharged residue such as alanine). Without wishing to be bound by any particular theory, amino acid substitutions of an antibody that result in changes of the pl of the antibody may improve solubility and/or the stability of the antibody. Appropriate amino acid substitutions can be selected for a particular antibody to achieve a desired pl. In some embodiments, a substitution is generated in an antibody to alter the pl. It is contemplated that the substitution(s) of the Fc region that result in altered binding to FcR (described herein) may also result in a change in the pl. In certain embodiments, substitution(s) of the Fc region are specifically chosen to effect both the desired alteration in FcR binding and any desired change in pl.

In some embodiments, an antibody lacking interchain cysteines has a Tm ranging from 65° C. to 120° C. In certain embodiments, an antibody lacking interchain cysteines has a Tm ranging from about 75° C. to about 120° C., or about 75° C. to about 85° C., or about 85° C. to about 95° C., or about 95° C. to about 105° C., or about 105° C. to about 115° C., or about 115° C. to about 120° C. In various embodiments, an antibody lacking interchain cysteines has a Tm ranging from 75° C. to 120° C., or 75° C. to 85° C., or 85° C. to 95° C., or 95° C. to 105° C., or 105° C. to 115° C., or 115° C. to 120° C. An antibody lacking interchain cysteines sometimes has a Tm of at least about 65° C., or at least about 70° C., or at least about 75° C., or at least about 80° C., or at least about 85° C., or at least about 90° C., or at least about 95° C., or at least about 100° C., or at least about 105° C., or at least about 110° C., or at least about 115° C., or at least about 120° C. In various embodiments, an antibody lacking interchain cysteines has a Tm of at least 65° C., or at least 70° C., or at least 75° C., or at least 80° C., or at least 85° C., or at least 90° C., or at least 95° C., or at least 100° C., or at least 105° C., or at least 110° C., or at least 115° C., or at least 120° C.

In various embodiments an antibody lacking interchain cysteines herein is stable at about 37° for five days or more. In some embodiments an antibody herein is stable in an animal for about 14 days or more.

### Antibody Conjugates

The use of antibody conjugates, i.e. immunoconjugates, for the local delivery of cytotoxic or cytostatic agents, i.e. drugs to kill or inhibit tumor cells in the treatment of cancer theoretically allows targeted delivery of the drug moiety to tumors, and intracellular accumulation therein, where systemic administration of these unconjugated drug agents may result in unacceptable levels of toxicity to normal cells as well as the tumor cells sought to be eliminated Efforts to design and refine antibody conjugates have focused on the selectivity of monoclonal antibodies (mAbs) as well as drug-linking and drug-releasing properties. Both polyclonal antibodies and monoclonal antibodies have been reported as useful in these strategies. Drugs used in these methods include daunomycin, doxorubicin, methotrexate, and vindesine.

Toxins used in antibody-toxin conjugates include bacterial toxins such as diphtheria toxin, plant toxins such as ricin, and gelonin, small molecule toxins such as geldanamycin, maytansinoids, and calicheamicin. The toxins may effect their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

Several antibody conjugates have been approved by the FDA or are in clinical trials. For instance, ZEVALIN® (ibritumomab tiuxetan, Biogen/Idec) is composed of amurine IgG1 kappa monoclonal antibody directed against the CD20 antigen found on the surface of normal and malignant B lymphocytes and 111 In or 90Y radioisotope bound by athiourea linker-chelator Although ZEVALIN® has activity against B cell non-Hodgkin's Lymphoma (NHL), administration results in severe and prolonged cytopenias in most patients. MYLOTARG® (gemtuzumab ozogamicin, Wyeth Pharmaceuticals), an antibody-drug conjugate composed of a human CD33 antibody linked to calicheamicin, was also approved in 2000 for the treatment of acute myeloid leukemia by injection. Cantuzumab mertansine (Immunogen, Inc.), an antibody-drug conjugate composed of the human C242 antibody linked via the disulfide linker SPP to the maytansinoid drug moiety, DM1, is advancing in clinical trials for the treatment of cancers that express CanAg, such as colon, pancreatic, gastric, and others. MLN-2704, an antibody-drug conjugate composed of composed of the anti-prostate specific membrane antigen (PSMA) monoclonal antibody linked to the maytansinoid drug moiety, DM1, is under development for the potential treatment of prostate tumors.

The auristatin peptides, auristatin E (AE) and monomethylauristatin (MMAE), synthetic analogs of dolastatin have been conjugated to: (i) chimeric monoclonal antibodies cBR96 (specific to Lewis Y on carcinomas); (ii) cAC10 which is specific to CD30 on hematological malignancies; (iii) anti-CD20 antibodies such as RITUXAN® for the treatment of CD20-expressing cancers and immune disorders; (iv) anti-EphB2R antibodies 2H9 and anti-IL-8 for treatment of colorectal cancer; (v) E-selectin antibody; and (vi) other anti-CD30 antibodies. Variants of auristatin E are disclosed in U.S. Pat. No. 5,767,237 and U.S. Pat. No. 6,124,431. Monomethyl auristatin E conjugated to monoclonal antibodies are disclosed in Senter et al, Proceedings of the American Association for Cancer Research, Volume 45, Abstract Number 623, presented Mar. 28, 2004. Auristatin analogs MMAE and MMAF have been conjugated to various antibodies (WO 2005/081711).

Provided herein, in some embodiments, is a method comprising the use of antibodies lacking interchain cysteines recombinantly fused or chemically conjugated (including both covalent and non-covalent conjugations) to a heterologous agent to generate a fusion protein as targeting moieties (hereinafter referred to as “antibody conjugates”). The heterologous agent may be linked to various regions of an antibody herein, including but not limited to the CH1, CH2, and CH3 domains. In some embodiments, a conjugated antibody herein comprises one or more hererologous agents. In certain embodiments, a conjugated antibody comprises an antibody homomultiplier conjugate.

The heterologous agent may be a polypeptide (or portion thereof, sometimes a polypeptide of at least 10, at least 20, at least 30, at least 40, at least 50, at least 60, at least 70, at least 80, at least 90 or at least 100 amino acids), nucleic acid, small molecule (less than 1000 daltons), or inorganic or organic compound. The fusion does not necessarily need to be direct, but may occur through linker sequences. Antibodies fused or conjugated to heterologous agents may be used in vivo to detect, treat, manage, or monitor the progression of a disorder using methods known in the art. In some embodiments, the disorder to be detected, treated, managed, or monitored is an autoimmune, inflammatory, infectious disease or cancer related disorder. Methods for fusing or conjugating polypeptides to antibody portions are known in the art.

Additional fusion proteins may be generated through the techniques of gene shuffling, motif-shuffling, exon-shuffling, and/or codon-shuffling (collectively referred to as “DNA shuffling”). DNA shuffling may be employed to alter the activities of antibodies lacking interchain cysteines herein (e.g., antibodies with higher affinities and lower dissociation rates). Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment may be recombined with one or more components, motifs, sections, parts, domains, fragments, etc. of one or more heterologous agents.

In an embodiment, antibodies lacking interchain cysteines herein or fragments or variants thereof are conjugated to a marker sequence, such as a peptide, to facilitate purification. In certain embodiments, the marker amino acid sequence is a hexahistidine peptide (SEQ ID NO: 19), such as the tag provided in a pQE vector (QIAGEN, Inc., 9259 Eton Avenue, Chatsworth, Calif., 91311), among others, many of which are commercially available. Hexa-histidine (SEQ ID NO: 19), for example, provides for convenient purification of the fusion protein. Other peptide tags useful for purification include, but are not limited to, the hemagglutinin “HA” tag, which corresponds to an epitope derived from the influenza hemagglutinin protein and the “flag” tag.

In some embodiments, antibodies herein are conjugated to a diagnostic or detectable agent. In some embodiments the agent is a detectable label. In certain embodiments the agent is an imaging agent. Such antibodies can be useful for monitoring or prognosing the development or progression of a disorder (such as, but not limited to cancer) as part of a clinical testing procedure, such as determining the efficacy of a particular therapy.

Such diagnosis and detection may accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, bioluminescent materials, such as but not limited to, luciferase, luciferin, and aequorin; radioactive materials, such as but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (166Ho), indium (¹¹⁵In, ¹¹³In, ¹¹¹In), iodine (¹³¹I, ¹²³I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), Rrhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti) tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

In certain embodiments, antibodies lacking interchain cysteines herein are conjugated to a therapeutic agent such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

In an embodiment, the cytotoxic agent is selected from the group consisting of an enediyne, a lexitropsin, a duocarmycin, a taxane, a puromycin, a dolastatin, a maytansinoid, and a vinca alkaloid. In some embodiments, the cytotoxic agent is paclitaxel, docetaxel, CC-1065, SN-38, topotecan, morpholino-doxorubicin, rhizoxin, cyanomorpholinodoxorubicin, dolastatin-10, echinomycin, combretastatin, calicheamicin, maytansine, DM-1, an auristatin or other dolastatin derivatives, such as auristatin E or auristatin F, AEB, AEVB, AEFP, MMAE (monomethylauristatin E), MMAF (monomethylauristatin F), eleutherobin ornetropsin. The synthesis and structure of auristatin E (dolastatin-10), and its derivatives are known in the art.

In some embodiments, the cytotoxic agent of an antibody conjugate herein is an anti-tubulin agent. Anti-tubulin agents are a well established class of cancer therapy compounds. Examples of anti-tubulin agents include, but are not limited to, taxanes (e.g., Taxol (paclitaxel), docetaxel), T67 (Tularik), vincas, and auristatins (e.g., auristatin E, AEB, AEVB, MMAE, MMAF, AEFP). Antitubulin agents included in this class are also: vinca alkaloids, including vincristine and vinblastine, vindesine and vinorelbine; taxanes such as paclitaxel and docetaxel and baccatin derivatives, epithilone A and B, nocodazole, 5-Fluorouracil and colcimid, estramustine, cryptophysins, cemadotin, maytansinoids, combretastatins, dolastatins, discodermolide and eleutherobin In more specific embodiments, the cytotoxic agent is selected from the group consisting of a vinca alkaloid, a podophyllotoxin, a taxane, a baccatin derivative, a cryptophysin, a maytansinoid, a combretastatin, and a dolastatin. In certain embodiments, the cytotoxic agent is vincristine, vinblastine, vindesine, vinorelbine, VP-16, camptothecin, paclitaxel, docetaxel, epithilone A, epithilone B, nocodazole, coichicine, colcimid, estramustine, cemadotin, discodermolide, maytansine, DM-1, an auristatin or other dolastatin derivatives, such as auristatin E or auristatin F, AEB, AEVB, AEFP, MMAE (monomethylauristatin E), MMAF (monomethylauristatin F), eleutherobin or netropsin.

In specific embodiments, the drug is a maytansinoid, a group of anti-tubulin agents. The drug is sometimes maytansine. The cytotoxic or cytostatic agent may be DM-1. Maytansine, a natural product, inhibits tubulin polymerization resulting in a mitotic block and cell death. Thus, the mechanism of action of maytansine appears to be similar to that of vincristine and vinblastine. Maytansine, however, is about 200 to 1,000-fold more cytotoxic in vitro than these vinca alkaloids. In an embodiment, the drug is an AEFP.

In some embodiments, the antibodies may be conjugated to other small molecule or protein toxins, such as, but not limited to abrin, brucine, cicutoxin, diphtheria toxin, batrachotoxin, botulism toxin, shiga toxin, endotoxin, tetanus toxin, pertussis toxin, anthrax toxin, cholera toxin falcarinol, fumonisin B1, fumonisin B2, afla toxin, maurotoxin, agitoxin, charybdotoxin, margatoxin, slotoxin, scyllatoxin, hefutoxin, calciseptine, taicatoxin, calcicludine, geldanamycin, gelonin, lotaustralin, ocratoxin A, patulin, ricin, strychnine, trichothecene, zearlenone, and tetradotoxin. Further examples of toxins, spacers, linkers, stretchers and the like, and their structures are known in the art.

As discussed herein, the compounds used for conjugation to the antibody conjugates herein can include conventional chemotherapeutics, such as doxorubicin, paclitaxel, carboplatin, melphalan, vinca alkaloids, methotrexate, mitomycin C, etoposide, and others. In addition, potent agents such CC-1065 analogues, calichiamicin, maytansine, analogues of dolastatin 10, rhizoxin, and palytoxin can be linked to the antibodies using the conditionally stable linkers to form potent immunoconjugates.

In certain embodiments, the cytotoxic or cytostatic agent is a dolastatin. In specific embodiments, the dolastatin is of the auristatin class. In some embodiments, the cytotoxic or cytostatic agent is MMAE. In various embodiments, the cytotoxic or cytostatic agent is AEFP. In some embodiments, the cytotoxic or cytostatic agent is MMAF.

In certain embodiments, antibodies herein are conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, cholera toxin, or diphtheria toxin; a protein such as tumor necrosis factor, alpha-interferon, beta-interferon, nerve growth factor, platelet derived growth factor, tissue plasminogen activator, an apoptotic agent, e.g., TNF-alpha, TNF-beta, AIM I, a thrombotic agent or an anti-angiogenic agent, e.g., angiostatin or endostatin; or, a biological response modifier such as, for example, a lymphokine (e.g., interleukin-1 (IL-1), interleukin-2 (IL-2), interleukin-4 (IL-4), interleukin-6 (IL-6), interleukin-7 (IL-7), interleukin-9 (IL-9), interleukin-15 (IL-15), interleukin-12 (IL-12), granulocyte macrophage colony stimulating factor (GMCSF), and granulocyte colony stimulating factor (G-CSF)), or a growth factor (e.g., growth hormone (GH)).

In some embodiments, antibodies herein are conjugated to a polypeptide that comprises poly arginine or poly-lysine residues. In certain embodiments, said polypeptide comprises 5, 6, 7, 8, 9, 10, 11, 12, 13, 14, 15, or more amino acid residues. In some embodiments, the poly-arginine polypeptide may comprise at least 5, 6, 7, 8, 9, 10, 11, 12, 13, 14, 15, or more arginine residues. In some embodiments, the poly-lysine polypeptide may comprise at least 5, 6, 7, 8, 9, 10, 11, 12, 13, 14, 15, or more lysine residues. In various embodiments, the polypeptide may comprise any combination of arginine and lysine residues.

In some embodiments, antibodies herein are conjugated to a therapeutic agent such as radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules, further discussed herein below, are known in the art. In some embodiments, antibodies herein are conjugated to a nucleic acid. The nucleic acid may be selected from the group consisting of DNA, RNA, short interfering RNA (siRNA), microRNA, hairpin or nucleic acid mimetics such as peptide nucleic acid. In certain embodiments the conjugated nucleic acid is at least 10, at least 20, at least 30, at least 40, at least 50, at least 60 at least 100, at least 200, at least 500, at least 1000, at least 5000 or more base pairs. The conjugated nucleic acid is sometimes single stranded. In various embodiments, the conjugated nucleic acid is double stranded.

In some embodiments, the conjugated nucleic acid encodes an open reading frame. In some embodiments, the open reading frame encoded by the conjugated nucleic acid corresponds to an apoptosis inducing protein, a viral protein, an enzyme, or a tumor suppressor protein. Techniques for delivery of such nucleic acids to cells are known in the art.

Techniques for conjugating therapeutic moieties to antibodies are known in the art. Moieties may be conjugated to antibodies by any method known in the art, including, but not limited to aldehyde/Schiff linkage, sulphydryl linkage, acid-labile linkage, cis-aconityl linkage, hydrazone linkage, and enzymatically degradable linkage. Additional techniques for conjugating therapeutic moieties to antibodies are also known. Methods for fusing or conjugating antibodies to polypeptide moieties are also known in the art. The fusion of an antibody to a moiety does not necessarily need to be direct, but may occur through linker sequences. Such linker molecules are known in the art.

Two approaches may be taken to minimize drug activity outside the cells that are targeted by the antibody conjugates herein: first, an antibody that binds to cell membrane receptor but not soluble receptor may be used, so that the drug, including drug produced by the actions of the prodrug converting enzyme, is concentrated at the cell surface of the activated lymphocyte. Another approach for minimizing the activity of drugs bound to the antibodies herein is to conjugate the drugs in a manner that would reduce their activity unless they are hydrolyzed or cleaved off the antibody. Such methods would employ attaching the drug to the antibodies with linkers that are sensitive to the environment at the cell surface of the activated lymphocyte (e.g., the activity of a protease that is present at the cell surface of the activated lymphocyte) or to the environment inside the activated lymphocyte the conjugate encounters when it is taken up by the activated lymphocyte (e.g., in the endosomal or, for example by virtue of pH sensitivity or protease sensitivity, in the lysosomal environment). Examples of linkers that can be used for conjugation of the antibodies herein are known in the art.

In an embodiment, the linker is an acid-labile hydrazone or hydrazide group that is hydrolyzed in the lysosome. In certain embodiments, drugs can be appended to antibodies through other acid-labile linkers, such as cis-aconitic amides, orthoesters, acetals and ketals. Such linkers are relatively stable under neutral pH conditions, such as those in the blood, but are unstable at below pH 5, the approximate pH of the lysosome.

In some embodiments, drugs are attached to the antibodies herein using peptide spacers that are cleaved by intracellular proteases. Target enzymes include cathepsins B and D and plasmin, all of which are known to hydrolyze dipeptide drug derivatives resulting in the release of active drug inside target cells. In intracellular proteolytic drug release the drug may be highly attenuated when conjugated and the serum stabilities of the conjugates may also be high. In some embodiments, the linker is a malonate linker, a maleimidobeiizoyl linker, or a 3′-N-amide analog.

As discussed above, antibody conjugates may be made by conjugating a compound or a drug to an antibody through a linker. Any linker that is known in the art may be used in the conjugates herein, e.g., bifunctional agents (such as dialdehydes or imidoesters) or branched hydrazone linkers.

In certain, non-limiting, embodiments herein, the linker region between the conjugate moiety and the antibody moiety is cleavable under certain conditions, where cleavage or hydrolysis of the linker releases the drug moiety from the antibody moiety. In some embodiments, the linker is sensitive to cleavage or hydrolysis under intracellular conditions.

In an embodiment, the linker region between the conjugate moiety and the antibody moiety is cleavable if the pH changes by a certain value or exceeds a certain value. In some embodiments herein, the linker is cleavable in the milieu of the lysosome, e.g., under acidic conditions (i.e., a pH of around 5-5.5 or less). In certain embodiments, the linker is a peptidyl linker that is cleaved by a peptidase or protease enzyme, including but not limited to a lysosomal protease enzyme, a membrane-associated protease, an intracellular protease, or an endosomal protease. The linker is sometimes at least two amino acids long, and may be at least three amino acids long. For example, a peptidyl linker that is cleavable by cathepsin-B (e.g., a Gly-Phe-Leu-Gly linker), a thiol-dependent protease that is highly expressed in cancerous tissue, can be used. Other such linkers are known in the art.

In other, non-mutually exclusive embodiments herein, the linker by which the antibody and compound of an antibody conjugate herein are conjugated promotes cellular internalization. In certain embodiments, the linker-drug moiety promotes cellular internalization. In certain embodiments, the linker is chosen such that the structure of the entire antibody conjugate promotes cellular internalization. In various embodiments, the linker is a thioether linker. In some embodiments, the linker is a hydrazone linker.

In certain embodiments, the linker is a disulfide linker. A variety of disulfide linkers are known in the art, including but not limited to those that can be formed using SATA (N-succinimidyl-S-acetylthioacetate), SPDP (N-succinimidyl-3-(2-pyridyldithio) propionate), SPDB (N-succinimidyl-3-(2-pyridyldithio)butyrate) and SMPT (Nsuccinimidyl-oxycarbonyl-alpha-methyl-alpha-(2-pyridyl-dithio)tol-uene). SPDB and SMPT. A variety of linkers that can be used with the compositions and methods herein are known in the art.

In some embodiments herein, the linker unit of an antibody conjugate links the cytotoxic or cytostatic agent (drug unit; -D) and the antibody unit (-A). In certain embodiments, the linker unit has the general formula:

-Ta-Ww--Yy-- where: i.
ii. -T- is a stretcher unit;
iii. a is 0 or 1;
iv. each --W-- is independently an amino acid unit;
v. w is independently an integer ranging from 2 to 12;
vi. —Y— is a spacer unit; and
vii. y is 0, 1 or 2.

The stretcher unit (-T-), when present, links the antibody unit to an amino acid unit (--W--). Useful functional groups that can be present on an antibody, either naturally or via chemical manipulation include, but are not limited to, sulfhydryl, amino, hydroxyl, the anomeric hydroxyl group of a carbohydrate, and carboxyl. Antibodies lacking interchain cysteines herein present at least one free sulfhydryl groups for conjugation. Other methods of introducing free sulfhydryl groups may involve the reduction of the intramolecular disulfide bonds of an antibody. Sulfhydryl groups can also be generated by reaction of an amino group of a lysine moiety of an antibody with 2-iminothiolane (Traut's reagent) or other sulfhydryl generating reagents.

The amino acid unit (—W—) links the stretcher unit (-T-) to the Spacer unit (—Y—) if the Spacer unit is present, and links the stretcher unit to the cytotoxic or cytostatic agent (Drug unit; D) if the spacer unit is absent.

In some embodiments, --Ww-- is a dipeptide, tripeptide, tetrapeptide, pentapeptide, hexapeptide, heptapeptide, octapeptide, nonapeptide, decapeptide, undecapeptide or dodecapeptide unit. The amino acid unit of the linker unit can be enzymatically cleaved by an enzyme including, but not limited to, a tumor-associated protease to liberate the drug unit (-D) which is protonated in vivo upon release to provide a cytotoxic drug (D).

In a an embodiment, the amino acid unit is a phenylalanine-lysine dipeptide (phe-lys or FK linker). In some embodiments, the amino acid unit is a valine-citrulline dipeptide (val-cit or VC linker).

The spacer unit (—Y—), when present, links an amino acid unit to the drug unit. Spacer units are of two general types: self-immolative and non self-immolative. A non self-immolative spacer unit is one in which part or all of the spacer unit remains bound to the drug unit after enzymatic cleavage of an amino acid unit from the antibody-linker-drug conjugate or the drug-linker compound. Examples of a non self-immolative spacer unit include, but are not limited to a (glycine-glycine) spacer unit and a glycine spacer unit. When an antibody-linker-drug conjugate herein containing a glycine-glycine spacer unit or a glycine spacer unit undergoes enzymatic cleavage via a tumor-cell associated-protease, a cancer-cell-associated protease or a lymphocyte-associated protease, a glycine-glycine-drug moiety or a glycine-drug moiety is cleaved from A-T-Ww--. To liberate the drug, an independent hydrolysis reaction may take place within the target cell to cleave the glycine drug unit bond.

Additional examples of self-immolative spacers include, but are not limited to aromatic compounds that are electronically equivalent to the PAB group such a 2-aminoimidazol-5-methanol derivatives. Spacers can be used that undergo facile cyclization upon amide bond hydrolysis, such as substituted and unsubstituted 4-aminobutyric acid amides, appropriately substituted ring systems, and 2-aminophenylpropionic acid amides. Elimination of amine-containing drugs that are substituted at the alpha-position of glycine are also examples of self-immolative spacer strategies that can be applied to the antibody-linker-drug conjugates herein.

### Methods for Conjugating a Heterologous Molecule to an Antibody

Heterologous molecules, such as those described herein may be efficiently conjugated to antibodies herein through the free thiol groups the engineered cysteine residues provide. In one aspect, the method provides for efficiently conjugating heterologus molecules to antibodies lacking interchain cysteines. In some embodiments the conjugation of a heterologus molecule may occur at a free thiol group provided by at least one engineered cysteine residue selected from one or more positions shown in Table 2. In certain embodiments, the conjugation of a heterologus molecule may occur at a free thiol group provided by at least one engineered cysteine residue selected from one or more positions shown in Table 3.

The engineering of non-naturally occurring cysteine residues into antibodies may alter the disulfide pairing of the heavy and light chains such that a naturally occurring cysteine residue which was part of a disulfide bond is liberated and presents a free thiol group capable of conjugation. In certain embodiments, the method comprises the efficient conjugation of a heterologus molecule to an antibody lacking interchain cysteines at a free thiol group not provided by at least one engineered cysteine residue selected from one or more positions shown in Table 2. In various embodiments, the method comprises the efficient conjugation of a heterologus molecule to an antibody lacking interchain cysteines at a free thiol group not provided by at least one engineered cysteine residue selected from one or more positions shown in the Table 3.

The presence of free thiol groups in antibodies may be determined by various art accepted techniques. The efficiency of conjugation of a heterologus molecule to an antibody may be determined by assessing the presence of free thiols remaining after the conjugation reaction. In certain embodiments, the method herein provides for efficiently conjugating a heterologus molecule to an antibody lacking interchain cysteines. In some embodiments, the conjugation efficiency is at least 5%, at least 10%, at least 15%, at least 20%, at least 25%, at least 30%, at least 35%, at least 40%, at least 45%, at least 50%, at least 55%, at least 60%, at least 70%, at least 75%, at least 80%, at least 85%, at least 90%, at least 95%, at least 98% or more as measured by the level of free thiol groups remaining after the conjugation reaction.

In some embodiments, the method herein provides for conjugating a heterologus molecule to an antibody where the antibody comprises at least one amino acid substitution, such that 2 or more free thiol groups are formed. In certain embodiments, the method comprises an antibody where the antibody comprises at least one amino acid substitution, such that at least 2, at least 4, at least 6, at least 8, at least 10, at least 12, at least 14, at least 16, at least 18, at least 20, at least 22, at least 24, at least 26, at least 28, at least 30, at least 32, at least 34, at least 36, at least 38, at least 40, or more free thiol groups are formed.

Antibodies herein capable of conjugation may contain free cysteine residues that comprise sulfhydryl groups that are blocked or capped. Such caps include proteins, peptides, ions and other materials that interact with the sulfhydryl group and prevent or inhibit conjugate formation. In some embodiments, antibodies herein may require uncapping prior to a conjugation reaction. In specific embodiments, antibodies herein are uncapped and display a free sulfhydryl group capable of conjugation. In specific embodiments, antibodies herein are subjected to an uncapping reaction that does not disturb or rearrange the naturally occurring disulfide bonds. In some embodiments, antibodies herein are subjected to an uncapping reaction as presented in PCT application PCT/US09/31294, filed Jan. 16, 2009.

In some embodiments, antibodies herein may be subjected to conjugation reactions where the antibody to be conjugated is present at a concentration of at least 1 mg/ml, at least 2 mg/ml, at least 3 mg/ml, at least 4 mg/ml, at least 5 mg/ml or higher.

### Methods of Using Antibody Conjugates

It is contemplated that the antibody conjugates herein may be used to treat various diseases or disorders, e.g. characterized by the over expression of a tumor antigen. In some embodiments an antibody conjugate herein inhibits tumor proliferation. In certain embodiments an antibody conjugate acts upon a subject in vivo. In certain embodiments an antibody conjugate acts in vitro.

In some embodiments an antibody conjugate is administered to a biological sample. A conjugate may contact a biological a sample, for example, by pipette, decantation, perfusion, injection, wash, bath, rotation, chromatography, or osmosis. In certain embodiments an antibody conjugate herein is cross-linked to a solid support, including but not limited to beads, and exposed to the sample. Bound antigen may be detected as known in the art, including but not limited to enzyme linked labels, secondary reactions, chromatographic, dye, fluorescence, and radiographic detection.

In certain embodiments, an antibody lacking interchain cysteines is conjugated to labels for purposes of diagnostics and other assays where the antibody and/or its associated ligand may be detected. A label conjugated to an antibody and used in the present methods and compositions described herein, is any chemical moiety, organic or inorganic, that exhibits an absorption maximum at wavelengths greater than 280 nm, and retains its spectral properties when covalently attached to an antibody. Labels include, without limitation, a chromophore, a fluorophore, a fluorescent protein, a phosphorescent dye, a tandem dye, a particle, a hapten, an enzyme and a radioisotope.

In certain embodiments, an antibody lacking interchain cysteines is conjugated to a fluorophore. As such, fluorophores used to label antibodies herein presented include, without limitation; a pyrene (including any of the corresponding derivative compounds), an anthracene, a naphthalene, an acridine, a stilbene, an indole or benzindole, an oxazole or benzoxazole, a thiazole or benzothiazole, a 4-amino-7-nitrobenz-2-oxa-1, 3-diazole (NBD), a cyanine (including any corresponding compounds), a carbocyanine (including any corresponding compounds), a carbostyryl, a porphyrin, a salicylate, an anthranilate, an azulene, a perylene, a pyridine, a quinoline, a borapolyazaindacene (including any corresponding compounds), a xanthene (including any corresponding compounds), an oxazine (including any corresponding compounds) or a benzoxazine, a carbazine (including any corresponding compounds), a phenalenone, a coumarin (including an corresponding compounds disclosed), a benzofuran (including an corresponding compounds) and benzphenalenone (including any corresponding compounds) and derivatives thereof. As used herein, oxazines include resorufins (including any corresponding compounds), aminooxazinones, diaminooxazines, and their benzo-substituted analogs.

In certain embodiments, the fluorophores conjugated to antibodies lacking interchain cysteines include xanthene (rhodol, rhodamine, fluorescein and derivatives thereof) coumarin, cyanine, pyrene, oxazine and borapolyazaindacene. In some embodiments, such fluorophores are sulfonated xanthenes, fluorinated xanthenes, sulfonated coumarins, fluorinated coumarins and sulfonated cyanines. Also included are dyes sold under the tradenames, and generally known as, Alexa Fluor, DyLight, Cy Dyes, BODIPY, Oregon Green, Pacific Blue, IRDyes, FAM, FITC, and ROX.

The choice of the fluorophore attached to an antibody lacking interchain cysteines will determine the absorption and fluorescence emission properties of the conjugated antibody. Physical properties of a fluorophore label that can be used for antibody and antibody bound ligands include, but are not limited to, spectral characteristics (absorption, emission and stokes shift), fluorescence intensity, lifetime, polarization and photo-bleaching rate, or combination thereof. All of these physical properties can be used to distinguish one fluorophore from another, and thereby allow for multiplexed analysis. In certain embodiments, the fluorophore has an absorption maximum at wavelengths greater than 480 nm. In some embodiments, the fluorophore absorbs at or near 488 nm to 514 nm (particularly suitable for excitation by the output of the argon-ion laser excitation source) or near 546 nm (particularly suitable for excitation by a mercury arc lamp). In some embodiments a fluorophore can emit in the NIR (near infra red region) for tissue or whole organism applications. Other desirable properties of the fluorescent label may include cell permeability and low toxicity, for example if labeling of the antibody is to be performed in a cell or an organism (e.g., a living animal).

In various embodiments, an enzyme is a label and is conjugated to a antibody lacking interchain cysteines. Enzymes are effective labels because amplification of the detectable signal can be obtained resulting in increased assay sensitivity. The enzyme itself often does not produce a detectable response but functions to break down a substrate when it is contacted by an appropriate substrate such that the converted substrate produces a fluorescent, colorimetric or luminescent signal. Enzymes amplify the detectable signal because one enzyme on a labeling reagent can result in multiple substrates being converted to a detectable signal. The enzyme substrate is selected to yield the measurable product, e.g. colorimetric, fluorescent or chemiluminescence. Such substrates are extensively used in the art and are known in the art.

In some embodiments, colorimetric or fluorogenic substrate and enzyme combination uses oxidoreductases such as horseradish peroxidase and a substrate such as 3,3′-diaminobenzidine (DAB) and 3-amino-9-ethylcarbazole (AEC), which yield a distinguishing color (brown and red, respectively). Other colorimetric oxidoreductase substrates that yield detectable products include, but are not limited to: 2,2-azino-bis(3-ethylbenzothiazoline-6-sulfonic acid) (ABTS), o-phenylenediamine (OPD), 3,3′,5,5′-tetramethylbenzidine (TMB), o-dianisidine, 5-aminosalicylic acid, 4-chloro-1-naphthol. Fluorogenic substrates include, but are not limited to, homovanillic acid or 4-hydroxy-3-methoxyphenylacetic acid, reduced phenoxazines and reduced benzothiazines, including Amplex® Red reagent and its variants and reduced dihydroxanthenes, including dihydrofluoresceins and dihydrorhodamines including dihydrorhodamine 123. Peroxidase substrates that are tyramides represent a unique class of peroxidase substrates in that they can be intrinsically detectable before action of the enzyme but are “fixed in place” by the action of a peroxidase in the process described as tyramide signal amplification (TSA). These substrates are extensively utilized to label targets in samples that are cells, tissues or arrays for their subsequent detection by microscopy, flow cytometry, optical scanning and fluorometry.

A colorimetric (and in some cases fluorogenic) substrate and enzyme combination sometimes uses a phosphatase enzyme such as an acid phosphatase, an alkaline phosphatase or a recombinant version of such a phosphatase in combination with a colorimetric substrate such as 5-bromo-6-chloro-3-indolyl phosphate (BCIP), 6-chloro-3-indolyl phosphate, 5-bromo-6-chloro-3-indolyl phosphate, p-nitrophenyl phosphate, or o-nitrophenyl phosphate or with a fluorogenic substrate such as 4-methylumbelliferyl phosphate, 6,8-difluoro-7-hydroxy-4-methylcoumarinyl phosphate (DiFMUP, U.S. Pat. No. 5,830,912) fluorescein diphosphate, 3-O-methylfluorescein phosphate, resorufin phosphate, 9H-(1,3-dichloro-9,9-dimethylacridin-2-one-7-yl) phosphate (DDAO phosphate), or ELF 97, ELF 39 or related phosphates.

Glycosidases, in particular beta-galactosidase, beta-glucuronidase and beta-glucosidase, are additional suitable enzymes. Appropriate colorimetric substrates include, but are not limited to, 5-bromo-4-chloro-3-indolyl beta-D-galactopyranoside (X-gal) and similar indolyl galactosides, glucosides, and glucuronides, o-nitrophenyl beta-D-galactopyranoside (ONPG) and p-nitrophenyl beta-D-galactopyranoside. In some embodiments, fluorogenic substrates include resorufin beta-D-galactopyranoside, fluorescein digalactoside (FDG), fluorescein diglucuronide and their structural variants, 4-methylumbelliferyl beta-D-galactopyranoside, carboxyumbelliferyl beta-D-galactopyranoside and fluorinated coumarin beta-D-galactopyranosides.

Additional enzymes include, but are not limited to, hydrolases such as cholinesterases and peptidases, oxidases such as glucose oxidase and cytochrome oxidases, and reductases for which suitable substrates are known.

Enzymes and their appropriate substrates that produce chemiluminescence are useful for some assays. These include, but are not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally productive.

In some embodiments, haptens such as biotin are also utilized as labels. Biotin is useful because it can function in an enzyme system to further amplify the detectable signal, and it can function as a tag to be used in affinity chromatography for isolation purposes. For detection purposes, an enzyme conjugate that has affinity for biotin is used, such as avidin-HRP. Subsequently a peroxidase substrate is added to produce a detectable signal.

Haptens also include hormones, naturally occurring and synthetic drugs, pollutants, allergens, affector molecules, growth factors, chemokines, cytokines, lymphokines, amino acids, peptides, chemical intermediates, nucleotides and the like.

In certain embodiments, fluorescent proteins are conjugated to the antibodies as a label. Examples of fluorescent proteins include green fluorescent protein (GFP) and the phycobiliproteins and the derivatives thereof. The fluorescent proteins, especially phycobiliprotein, are useful for creating tandem dye labeled labeling reagents. These tandem dyes comprise a fluorescent protein and a fluorophore for the purposes of obtaining a larger stokes shift where the emission spectra is farther shifted from the wavelength of the fluorescent protein's absorption spectra. This may be effective for detecting a low quantity of a target in a sample where the emitted fluorescent light is maximally optimized, in other words little to none of the emitted light is reabsorbed by the fluorescent protein. For this to work, the fluorescent protein and fluorophore function as an energy transfer pair where the fluorescent protein emits at the wavelength that the fluorophore absorbs at and the fluorphore then emits at a wavelength farther from the fluorescent proteins than could have been obtained with only the fluorescent protein. A functional combination may be phycobiliproteins and sulforhodamine fluorophores or sulfonated cyanine fluorophores as known in the art. The fluorophore sometimes functions as the energy donor and the fluorescent protein is the energy acceptor.

In certain embodiments, the label is a radioactive isotope. Examples of suitable radioactive materials include, but are not limited to, iodine (.sup.121I, .sup.123I, .sup.125I, .sup.131I), carbon (.sup.14C), sulfur (.sup.35S), tritium (.sup.3H), indium (.sup.111In, .sup.112In, .sup.113mIn, .sup.115mIn), technetium (.sup.99Tc, .sup.99mTc), thallium (.sup.201Ti), gallium (.sup.68Ga, .sup.67Ga), palladium (.sup.103Pd), molybdenum (.sup.99Mo), xenon (.sup.135Xe), fluorine (.sup.18F), .sup.153Sm, .sup.177Lu, .sup.159Gd, .sup.149Pm, .sup.140La, .sup.175Yb, .sup.166Ho, .sup.90Y, .sup.47Sc, .sup.186Re, .sup.188Re, .sup.142Pr, .sup.105Rh, and .sup.97Ru.

Diagnostic Methods of Use

In certain embodiments, antibodies lacking interchain cysteines, conjugates and compositions herein presented may be used in vivo and/or in vitro for diagnosing diseases associated with the FlexiMab antibody or the conjugated molecule. This can be achieved, for example, by contacting a sample to be tested, optionally along with a control sample, with the antibody under conditions that allow for formation of a complex between the antibody or conjugate herein and the molecule of interest. Complex formation is then detected (e.g., using an ELISA). When using a control sample along with the test sample, complex is detected in both samples and any statistically significant difference in the formation of complexes between the samples is indicative of the presence of the molecule of interest in the test sample.

In some embodiments, the technology herein provides a method of determining the presence of a molecule of interest in a sample suspected of containing such a molecule, the method comprising exposing the sample to an antibody lacking interchain cysteines or conjugate, and determining binding of the antibody or conjugate to the molecule of interest in the sample where binding of the antibody or conjugate to the molecule of interest in the sample is indicative of the presence of the molecule of interest in the sample. In some embodiments, the sample is a biological sample. In certain embodiments, the biological sample is from a mammal experiencing or suspected of experiencing disease or disorder associated with the molecule of interest.

In certain embodiments, an antibody lacking interchain cysteines or conjugate may be used to detect the overexpression or amplification of a molecule of interest using an in vivo diagnostic assay. In some embodiments, an antibody lacking interchain cysteines or conjugate is added to a sample where the antibody or conjugate binds the molecule of interest to be detected and is tagged with a detectable label (e.g. a radioactive isotope or a fluorescent label) and externally scanning the patient for localization of the label.

FISH assays such as the INFORM™ (sold by Ventana, Ariz.) or PATHVISION™ (Vysis, Ill.) may be carried out on formalin-fixed, paraffin-embedded tissue to determine the extent (if any) of overexpression of a molecule of interest in the tumor.

In certain embodiments, an antibody lacking interchain cysteines or conjugate may be used in a method of diagnosing a cell proliferative disorder associated with an increase in cells expressing a molecule of interest. In some embodiments, the method comprises contacting test cells in a biological sample with an antibody lacking interchain cysteines or conjugate; determining the level of a molecule of interest in test cells in the sample by detecting binding of an antibody lacking interchain cysteines or conjugate; and comparing the level of antibody bound to cells in a control sample, where the level of antibody bound is normalized to the number molecule of interest expressing cells in the test and control samples, and where a higher level of antibody bound in the test sample as compared to the control sample indicates the presence of a cell proliferative disorder associated with cells expressing the molecule of interest.

In certain embodiments, an antibody lacking interchain cysteines or conjugate may be used in a method of detecting soluble molecule of interest in blood or serum. In some embodiments, the method comprises contacting a test sample of blood or serum from a mammal suspected of experiencing a disorder associated with a molecule of interest with an antibody lacking interchain cysteines of conjugate herein and detecting an increase in soluble molecule of interest in the test sample relative to a control sample of blood or serum from a normal mammal. In some embodiments, the method of detecting is useful as a method of diagnosing a disorder associated with an increase in soluble molecule of interest in blood or serum of a mammal.

Treatment Methods of Use

In various embodiments the antibody conjugate is administered to cells, for example cancer cells. The biological effect of the antibody conjugate may be observed, including but not limited to cell, death, cell proliferation inhibition, lack of effect, changes in cell morphology, and changes in cellar growth pattern. In some embodiments the antibody conjugate comprises a detectable label as described above. In certain embodiments the label indicates the location of the tumor antigen within the cell.

In certain embodiments an antibody conjugate is administered to a subject in need of treatment. In various embodiments a conjugate carries a drug or toxin targeted to a tumor antigen. The conjugate sometimes carries a detectable label by which the antigen may be identified or localized. Some embodiments comprise detection of the biological effect of the antibody conjugate. In certain embodiments the condition of the subject may be monitored. The medical dose may be adjusted in response to monitoring.

Exemplary conditions or hyperproliferative disorders include benign or malignant tumors, leukemia and lymphoid malignancies. Others include neuronal, glial, astrocytal, hypothalamic, glandular, macrophagal, epithelial, endothelial, and stromal malignancies. Other cancers or hyperproliferative disorders include: cancers of the head, neck, eye, mouth, throat, esophagus, chest, skin, bone, lung, colon, rectum, colorectal, stomach, spleen, kidney, skeletal muscle, subcutaneous tissue, metastatic melanoma, endometrial, prostate, breast, ovaries, testicles, thyroid, blood, lymph nodes, kidney, liver, pancreas, brain, or central nervous system. Examples of cancers that can be prevented, managed, treated or ameliorated in accordance with the methods herein include, but are not limited to, cancer of the head, neck, eye, mouth, throat, esophagus, chest, bone, lung, colon, rectum, stomach, prostate, breast, ovaries, kidney, liver, pancreas, and brain. Additional cancers include, but are not limited to, the following: leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple mycloma, nonsecretory myeloma, osteosclerotic myeloma, plasma cell leukemia, solitary plasmacytoma and extramedullary plasmacytoma, Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/or ureter); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesotheliorna, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas.

It is also contemplated that cancers caused by aberrations in apoptosis can also be treated by the methods and compositions herein. Such cancers may include, but not be limited to, follicular lymphomas, carcinomas with p53 mutations, hormone dependent tumors of the breast, prostate and ovary, and precancerous lesions such as familial adenomatous polyposis, and myelodysplastic syndromes.

The proteins herein and compositions comprising the same are useful for many purposes, for example, as therapeutics against a wide range of chronic and acute diseases and disorders including, but not limited to, autoimmune and/or inflammatory disorders, which include Sjogren's syndrome, rheumatoid arthritis, lupus psoriasis, atherosclerosis, diabetic and other retinopathies, retrolental fibroplasia, age-related macular degeneration, neovascular glaucoma, hemangiomas, thyroid hyperplasias (including Grave's disease), corneal and other tissue transplantation, and chronic inflammation, sepsis, rheumatoid arthritis, peritonitis, Crohn's disease, reperfusion injury, septicemia, endotoxic shock, cystic fibrosis, endocarditis, psoriasis, arthritis (e.g., psoriatic arthritis), anaphylactic shock, organ ischemia, reperfusion injury, spinal cord injury and allograft rejection.

Examples of autoimmune and/or inflammatory disorders include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, Sjogren's syndrome, psoriasis, atherosclerosis, diabetic and other retinopathies, retrolental fibroplasia, age-related macular degeneration, neovascular glaucoma, hemangiomas, thyroid hyperplasias (including Grave's disease), corneal and other tissue transplantation, and chronic inflammation, sepsis, rheumatoid arthritis, peritonitis, Crohn's disease, reperfusion injury, septicemia, endotoxic shock, cystic fibrosis, endocarditis, psoriasis, arthritis (e.g., psoriatic arthritis), anaphylactic shock, organ ischemia, reperfusion injury, spinal cord injury and allograft rejection. autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, discoid lupus, essential mixed cryoglobulinemia, fibromyalgia-fibromyositis, glomerulonephritis, Guillain-Barre, Hashimoto's thyroiditis, idiopathic pulmonary fibrosis, idiopathic thrombocytopenia purpura (ITP), IgA neuropathy, juvenile arthritis, lichen planus, lupus erythematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteristis/giant cell arteritis, ulcerative colitis, uveitis, vasculitides such as dermatitisherpetiformis vasculitis, vitiligo, and Wegener's granulomatosis.

Examples of inflammatory disorders include, but are not limited to, asthma, encephilitis, inflammatory bowel disease, chronic obstructive pulmonary disease (COPD), allergic disorders, septic shock, pulmonary fibrosis, undifferentitated spondyloarthropathy, undifferentiated arthropathy, arthritis, inflammatory osteolysis, and chronic inflammation resulting from chronic viral or bacteria infections.

The compositions and methods herein can be used with one or more conventional therapies that are used to prevent, manage or treat the above diseases. Also provided, in some embodiments are methods of using antibodies and/or antibody conjugates to inactivate various infectious agents such as viruses, fungi, eukaryotic microbes, and bacteria. In some embodiments the antibodies or antibody conjugates herein may be used to inactivate RSV, hMPV, PIV, or influenza viruses. In some embodiments, the antibodies and/or antibody conjugates herein may be used to inactivate fungal pathogens, such as, but not limited to members of Naegleria, Aspergillus, Blastomyces, Histoplasma, Candida or Tinea genera. In some embodiments, the antibodies and/or antibody conjugates herein may be used to inactivate eukaryotic microbes, such as, but not limited to members of Giardia, Toxoplasma, Plasmodium, Trypanosoma, and Entamoeba genera. In some embodiments, the antibodies and/or antibody conjugates herein may be used to inactivate bacterial pathogens, such as but not limited to members of Staphylococcus, Streptococcus, Pseudomonas, Clostridium, Borrelia, Vibro and Neiserria genera.

The antibodies and/or antibody conjugates herein and compositions comprising the same are useful for many purposes, for example, as therapeutics against a wide range of chronic and acute diseases and disorders including, but not limited to, infectious disease, including viral, bacterial and fungal diseases. Examples of viral pathogens include but are not limited to: adenovirdiae (e.g., mastadenovirus and aviadenovirus), herpesviridae (e.g., herpes simplex virus 1, herpes simplex virus 2, herpes simplex virus 5, and herpes simplex virus 6), leviviridae (e.g., levivirus, enterobacteria phase MS2, allolevirus), poxviridae (e.g., chordopoxvirinae, parapoxvirus, avipoxvirus, capripoxvirus, leporiipoxvirus, suipoxvirus, molluscipoxvirus, and entomopoxvirinae), papovaviridae (e.g., polyomavirus and papillomavirus), paramyxoviridae (e.g., mparamyxovirus, parainfluenza virus 1, mobillivirus (e.g., measles virus), rubulavirus (e.g., mumps virus), pneumonovirinae (e.g., pneumovirus, human respiratory synctial virus), and metapneumovirus (e.g., avian pneumovirus and human metapneumovirus)), picornaviridae (e.g., enterovirus, rhinovirus, hepatovirus (e.g., human hepatitis A virus), cardiovirus, and apthovirus), reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLVHTLV retroviruses, lentivirus (e.g. human immunodeficiency virus 1 and human immunodeficiency virus 2), spumavirus), flaviviridae (e.g., hepatitis C virus), hepadnaviridae (e.g., hepatitis B virus), togaviridae (e.g., alphavirus (e.g., sindbis virus) and rubivirus (e.g., rubella virus)), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus).

Examples of bacterial pathogens include but are not limited to: the Aquaspirillum family, Azospirillum family, Azotobacteraceae family, Bacteroidaceae family, Bartonella species, Bdellovibrio family, Campylobacter species, Chlamydia species (e.g., Chlamydia pneumoniae), clostridium, Enterobacteriaceae family (e.g., Citrobacter species, Edwardsiella, Enterobacter aerogenes, Erwinia species, Escherichia coli, Hafnia species, Klebsiella species, Morganella species, Proteus vulgaris, Providencia, Salmonella species, Serratia marcescens, and Shigella flexneri), Gardinella family, Haemophilus influenzae, Halobacteriaceae family, Helicobacter family, Legionallaceae family, Listeria species, Methylococcaceae family, mycobacteria (e.g., Mycobacterium tuberculosis), Neisseriaceae family, Oceanospirillum family, Pasteurellaceae family, Pneumococcus species, Pseudomonas species, Rhizobiaceae family, Spirillum family, Spirosomaceae family, Staphylococcuss (e.g., methicillin resistant Staphylococcus aureus and Staphylococcus pyrogenes), Streptococcus (e.g., Streptococcus enteritidis, Streptococcus fasciae, and Streptococcus pneumoniae), Vampirovibr Helicobacter family, and Vampirovibrio family.

Examples of fungal pathogens include, but are not limited to: Absidia species (e.g., Absidia corymbifera and Absidia ramosa), Aspergillus species, (e.g., Aspergillus flavus, Aspergillus fumigatus, Aspergillus nidulans, Aspergillus niger, and Aspergillus terreus), Basidiobolus Ranarum, Blastomyces dermatitidis, Candida species (e.g., Candida albicans, Candida glabrata, Candida kerr, Candida krusei, Candida parapsilosis, Candida pseudotropicalis, Candida quillermondii, Candida rugosa, Candida stellatoidea, and Candida tropicalis), Coccidioides immitis, Conidiobolus species, Cryptococcus neoforms, Cunninghamella species, dermatophytes, Histoplasma capsulatum, Microsporum gypseum, Mucor pusillus, Paracoccidioides brasiliensis, Pseudallescheria boydii, Rhinosporidium seeberi, Pneumocystis carinii, Rhizopus species (e.g., Rhizopus arrhizus, Rhizopus oryzae, and Rhizopus microsporus), Saccharomyces species, Sporothrix schenckii, zygomycetes, and classes such as Zygomycetes, Ascomycetes, the Basidiomycetes, Deuteromycetes, and Oomycetes.

Provided also, in some embodiments, are methods of using antibodies to deplete a cell population. In an embodiment, methods herein may be used in the depletion of the following cell types: eosinophil, basophil, neutrophil, T cell, B cell, mast cell, monocytes, endothelial cell and tumor cell.

In certain embodiments, the antibodies herein and conjugates thereof may also be useful in the diagnosis and detection of diseases of symptoms thereof. In some embodiments, the compositions herein may be useful in the monitoring of disease progression. In various embodiments, the compositions herein may be useful in the monitoring of treatment regimens. In certain embodiments, the compositions herein are useful for diagnosis in an ex vivo application, such as a diagnostic kit.

The compositions herein may be useful in the visualization of target antigens. In some embodiments, the target antigens are cell surface receptors that internalize. In certain embodiments, the target antigen is an intracellular antigen. In some embodiments the target is an intranuclear antigen.

In some embodiments, the antibodies or antibody-drug conjugates herein once bound, internalize into cells where internalization is at least about 10%, at least about 20%, at least about 30%, at least about 40%, at least about 50%, at least about 60%, at least about 70%, at least about 80%, or at least about 90%, at least about 100%, at least about 110%, at least about 130%, at least about 140%, at least about 150%, at least about 160%, or at least about 170% more than control antibodies as described herein.

In certain embodiments, the antibodies herein once bound, internalize into cells where internalization is 1-10%, 10-20%, 20-30%, 30-40%, 40-50%, 50-60%, 60-70%, 70-80%, 80-90%, 90-100%, 100-110%, 110-120%, 120-130%, 130-140%, 140-150%, 150-160%, 160-170% more than control antibodies as described herein.

Various embodiments, the antibodies herein once bound, internalize into cells where internalization is 1-10%, 10-20%, 20-30%, 30-40%, 40-50%, 50-60%, 60-70%, 70-80%, 80-90%, 90-100%, 100-110%, 110-120%, 120-130%, 130-140%, 140-150%, 150-160%, 160-170% more than control antibodies as determined by the internalization assay using a secondary antibody.

### Antibody Therapeutics

Pharmaceutical Compositions

Provided herein, in some embodiments, is a composition, for non-limiting example, a pharmaceutical composition, containing one or a combination of antibodies, or antibody conjugates herein, formulated together with a pharmaceutically acceptable carrier. Such compositions may include one or a combination of, for example, but not limited to two or more different antibodies herein. For example, a pharmaceutical composition herein may comprise a combination of antibodies that bind to different epitopes on the target antigen or that have complementary activities.

To prepare pharmaceutical or sterile compositions including an antibody or antibody conjugate herein, the antibody/antibody conjugate can be mixed with a pharmaceutically acceptable carrier or excipient. Formulations of therapeutic and diagnostic agents can be prepared by mixing with physiologically acceptable carriers, excipients, or stabilizers in the form of, e.g., lyophilized powders, slurries, aqueous solutions, lotions, or suspensions.

Pharmaceutical compositions herein also can be administered in combination therapy, such as, combined with other agents. For example, the combination therapy can include an antibody herein combined with at least one other therapy where the therapy may be surgery, immunotherapy, chemotherapy, radiation treatment, or drug therapy.

The pharmaceutical compounds herein may include one or more pharmaceutically acceptable salt. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N methylglucamine, chloroprocaine, choline, diethanolamine, ethylenediamine, procaine and the like.

A pharmaceutical composition herein also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oilsoluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and non-aqueous carriers that may be employed in the pharmaceutical compositions herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutical compositions may be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it may be suitable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, appropriate methods of preparation include vacuum drying and freeze-drying (lyophilization) that yield a powder of the active ingredient plus any additional desired ingredient from a previously sterile-filtered solution thereof.

In one embodiment the compositions herein are pyrogen-free formulations which are substantially free of endotoxins and/or related pyrogenic substances. Endotoxins include toxins that are confined inside a microorganism and are released when the microorganisms are broken down or die. Pyrogenic substances also include fever inducing, thermostable substances (glycoproteins) from the outer membrane of bacteria and other microorganisms. Both of these substances can cause fever, hypotension and shock if administered to humans. Due to the potential harmful effects, even low amounts of endotoxins may be appropriately removed from intravenously administered pharmaceutical drug solutions. The Food & Drug Administration (“FDA”) has set an upper limit of 5 endotoxin units (EU) per dose per kilogram body weight in a single one hour period for intravenous drug applications. When therapeutic proteins are administered in amounts of several hundred or thousand milligrams per kilogram body weight even trace amounts of endotoxin may appropriately be removed. In an embodiment, endotoxin and pyrogen levels in the composition are less then 10 EU/mg, less then 5 EU/mg, less then 1 EU/mg, less then 0.1 EU/mg, less then 0.01 EU/mg, or less then 0.001 EU/mg. In certain embodiments, endotoxin and pyrogen levels in the composition are less then about 10 EU/mg, less then about 5 EU/mg, less then about 1 EU/mg, or less then about 0.1 EU/mg, less then about 0.01 EU/mg, or less then about 0.001 EU/mg.

In some embodiments, a method comprises administering a composition where said administration is oral, parenteral, intramuscular, intranasal, vaginal, rectal, lingual, sublingual, buccal, intrabuccal, intravenous, cutaneous, subcutaneous or transdermal.

In certain embodiments, a method further comprises administering a composition in combination with other therapies, such as surgery, chemotherapy, hormonal therapy, biological therapy, immunotherapy or radiation therapy.

Dosing and Administration

To prepare pharmaceutical or sterile compositions including an antibody or antibody conjugate herein, the antibody/antibody conjugate is mixed with a pharmaceutically acceptable carrier or excipient. Formulations of therapeutic and diagnostic agents can be prepared by mixing with physiologically acceptable carriers, excipients, or stabilizers in the form of, e.g., lyophilized powders, slurries, aqueous solutions, lotions, or suspensions.

Selecting an administration regimen for a therapeutic depends on several factors, including the serum or tissue turnover rate of the entity, the level of symptoms, the immunogenicity of the entity, and the accessibility of the target cells in the biological matrix. In certain embodiments, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules is available in the art.

Determination of the appropriate dose may be made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Actual dosage levels of the active ingredients in the pharmaceutical compositions herein may be varied so as to obtain an amount of the active ingredient which is effective to achieve the desired therapeutic response for a particular patient, composition, and mode of administration, without being toxic to the patient. The selected dosage level may depend upon a variety of pharmacokinetic factors including the activity of the particular compositions herein employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors known in the medical arts.

Compositions comprising antibodies or antibody conjugates herein can be provided by continuous infusion, or by doses at intervals of, e.g., one day, one week, or 1-7 times per week. Doses may be provided intravenously, subcutaneously, topically, orally, nasally, rectally, intramuscular, intracerebrally, or by inhalation. A specific dose protocol is one involving the maximal dose or dose frequency that avoids significant undesirable side effects. A total weekly dose may be at least 0.05 μg/kg body weight, at least 0.2 μg/kg, at least 0.5 μg/kg, at least 1 μg/kg, at least 10 μg/kg, at least 100 μg/kg, at least 0.2 mg/kg, at least 1.0 mg/kg, at least 2.0 mg/kg, at least 10 mg/kg, at least 25 mg/kg, or at least 50 mg/kg. The dose may be at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg. The doses administered to a subject may number at least 1, 2, 3, 4, 5, 6, 7, 8, 9, 10, 11, or 12, or more.

For antibodies or antibody conjugates herein, the dosage administered to a patient may be 0.0001 mg/kg to 100 mg/kg of the patient's body weight. The dosage may be between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight.

The dosage of the antibodies or antibody conjugates herein may be calculated using the patient's weight in kilograms (kg) multiplied by the dose to be administered in mg/kg. The dosage of the antibodies herein may be 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight.

Unit dose of the antibodies or antibody conjugates herein may be 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

The dosage of the antibodies or antibody conjugates herein may achieve a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml in a subject. Alternatively, the dosage of the antibodies herein may achieve a serum titer of at least 0.1 μg/ml, at least 0.5 μg/ml, at least 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml in the subject.

Doses of antibodies or antibody conjugates herein may be repeated and the administrations may be separated by at least 1 day, 2 days, 3 days, 5 days, 10 days, 15 days, 30 days, 45 days, 2 months, 75 days, 3 months, or at least 6 months.

An effective amount for a particular patient may vary depending on factors such as the condition being treated, the overall health of the patient, the method route and dose of administration and the severity of side affects.

The route of administration may be by, e.g., topical or cutaneous application, injection or infusion by intravenous, intraperitoneal, intracerebral, intramuscular, intraocular, intraarterial, intracerebrospinal, intralesional, or by sustained release systems or an implant. Where necessary, the composition may also include a solubilizing agent and a local anesthetic such as lidocaine to ease pain at the site of the injection. In addition, pulmonary administration can also be employed, e.g., by use of an inhaler or nebulizer, and formulation with an aerosolizing agent. In an embodiment, an antibody, combination therapy, or a composition herein is administered using Alkermes AIR® pulmonary drug delivery technology (Alkermes, Inc., Cambridge, Mass.).

A composition herein may also be administered via one or more routes of administration using one or more of a variety of methods known in the art. As will be appreciated by the skilled artisan, the route and/or mode of administration may vary depending upon the desired results. Selected routes of administration for antibodies herein include intravenous, intramuscular, intradermal, intraperitoneal, subcutaneous, spinal or other parenteral routes of administration, for example by injection or infusion. Parenteral administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition herein can be administered via a non-parenteral route, such as a topical, epidermal or mucosal route of administration, for example, intranasally, orally, vaginally, rectally, sublingually or topically.

If the antibodies herein or conjugates thereof are administered in a controlled release or sustained release system, a pump may be used to achieve controlled or sustained release. Polymeric materials can be used to achieve controlled or sustained release of the therapies herein. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In an embodiment, the polymer used in a sustained release formulation is inert, free of leachable impurities, stable on storage, sterile, and biodegradable.

A controlled or sustained release system can be placed in proximity of the prophylactic or therapeutic target, thus requiring only a fraction of the systemic dose. Any technique known in the art can be used to produce sustained release formulations comprising one or more antibodies herein or conjugates thereof.

If the antibody or antibody conjugate herein is administered topically, it can be formulated in the form of an ointment, cream, transdermal patch, lotion, gel, shampoo, spray, aerosol, solution, emulsion, or other form known in the art. For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations where the active ingredient, in some instances, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are known in the art.

If the compositions comprising antibodies or antibody conjugates are administered intranasally, it can be formulated in an aerosol form, spray, mist or in the form of drops. In particular, prophylactic or therapeutic agents for use as provided herein can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

Methods for co-administration or treatment with a second therapeutic agent, e.g., a cytokine, steroid, chemotherapeutic agent, antibiotic, or radiation, are known in the art. An effective amount of therapeutic may decrease the symptoms by at least 10%; by at least 20%; at least about 30%; at least 40%, or at least 50%.

Additional therapies (e.g., prophylactic or therapeutic agents), which can be administered in combination with the antibodies herein or conjugates thereof, may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart from the antibodies herein. The two or more therapies may be administered within one patient visit or on separate visits.

The antibodies or antibody conjugates herein and the other therapies may be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the antibodies and antibody conjugates herein can be formulated to ensure proper distribution in vivo. For example, the blood brain barrier (BBB) excludes many highly hydrophilic compounds. To ensure that the therapeutic compounds herein cross the BBB (if desired), they can be formulated, for example, in liposomes. Methods of manufacturing liposomes are known in the art. The liposomes may comprise one or more moieties which are selectively transported into specific cells or organs, thus enhance targeted drug delivery. Exemplary targeting moieties include folate or biotin, mannosides, antibodies, surfactant, and protein A receptor.

Also provided, in some embodiments, are protocols for the administration of pharmaceutical composition comprising antibodies or antibody conjugates herein alone or in combination with other therapies to a subject in need thereof. The therapies (e.g., prophylactic or therapeutic agents) of the combination therapies herein can be administered concomitantly or sequentially to a subject. The therapy (e.g., prophylactic or therapeutic agents) of the combination therapies herein can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

The therapies (e.g., prophylactic or therapeutic agents) of the combination therapies herein can be administered to a subject concurrently. The term “concurrently” is not limited to the administration of therapies (e.g., prophylactic or therapeutic agents) at exactly the same time, but rather it is meant that a pharmaceutical composition comprising antibodies or antibody conjugates herein are administered to a subject in a sequence and within a time interval such that the antibodies herein or conjugates thereof can act together with the other therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route. In various embodiments, the therapies (e.g., prophylactic or therapeutic agents) are administered to a subject less than 15 minutes, less than 30 minutes, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, 24 hours apart, 48 hours apart, 72 hours apart, or 1 week apart. In certain embodiments, two or more therapies (e.g., prophylactic or therapeutic agents) are administered to a within the same patient visit.

The prophylactic or therapeutic agents of the combination therapies may be administered to a subject in the same pharmaceutical composition. In some embodiments, the prophylactic or therapeutic agents of the combination therapies can be administered concurrently to a subject in separate pharmaceutical compositions. The prophylactic or therapeutic agents may be administered to a subject by the same or different routes of administration.

### Examples

The examples set forth below illustrate certain embodiments and do not limit the technology.

### Example 1—General Cloning Procedures

DNA manipulations were carried out according to standard protocols with reagents purchased from Invitrogen (Carlsbad, Calif.), New England Biolabs (Ipswich, Mass.), Qiagen (Valentia, Calif.) and Fermentas (Glen Burnie, Md.). All polymerase chain reactions (PCRs) were carried out using Platinum® Taq DNA Polymerase High Fidelity (Invitrogen). The amplified PCR fragments, analyzed using E-gels (Invitrogen), were digested with the appropriate restriction enzymes and purified using preparative agarose gel (Sigma Aldrich, St. Louis, Mo.). The purified DNA fragments were ligated into a similarly prepared mammalian expression vector pOE (Medlmmune) using reagents and protocols supplied by Invitrogen and New England Biolabs. In this expression vector both light and heavy chains are under the control of respective CMV immediate/early enhancer/promoter with a multiple cloning site and a SV40 poly(A) signal. The ligation mixtures were chemically transformed into Escherichia coli Stb13™ (Invitrogen). Recombinant clones were identified by either colony PCR, using primers complementary to the 5′ and 3′ ends of the recombinant gene inserts, or by restriction digestion analysis using restriction enzymes that specifically cleave correct clones. All recombinant clones were further verified by DNA sequence analysis using Dye Terminator Cycle Sequencing Kits with AmpliTap (Applied Biosystems, Foster City, Calif.).

### Example 2—Expression, Purification and Monomeric Content of mAb in a Conventional Format and FlexiMab

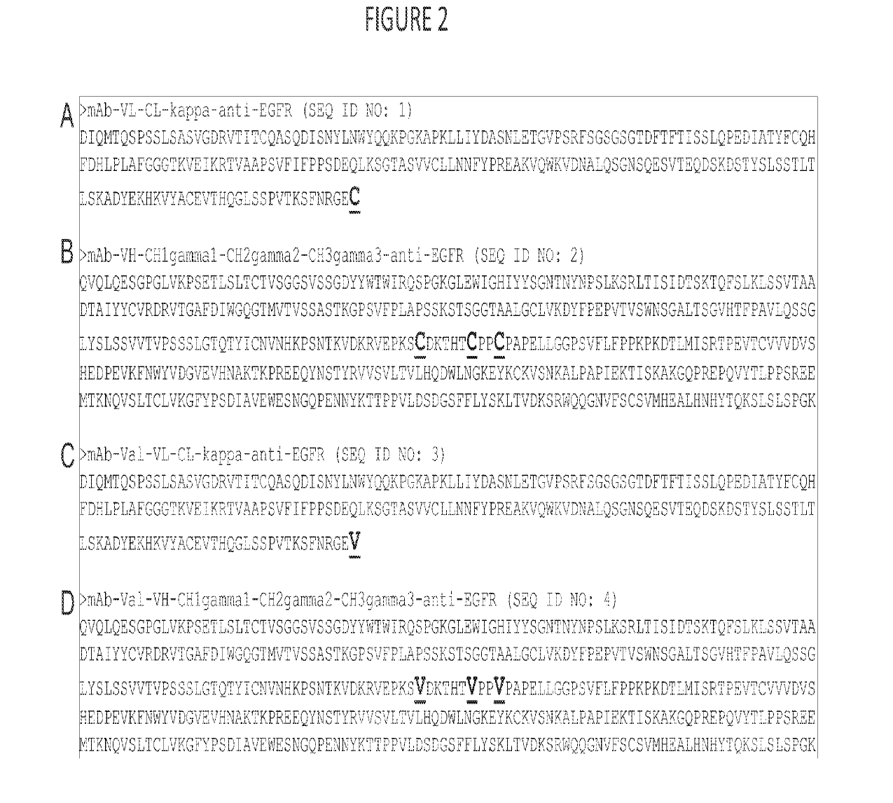

DNA encoding for mAb and FlexiMab were transfected and proteins were expressed in HEK293F cells cultivated in Invitrogen's Freestyle™ media. The FexiMab used in these examples is a “mab-Val,” which is a FlexiMab antibody in which interchain cysteines have been substituted with valine (e.g., FIG. 2, panels C and D). The term “mAb” refers to the counterpart antibody that does not include such cysteine substitutions (e.g., FIG. 2, panels A and B).

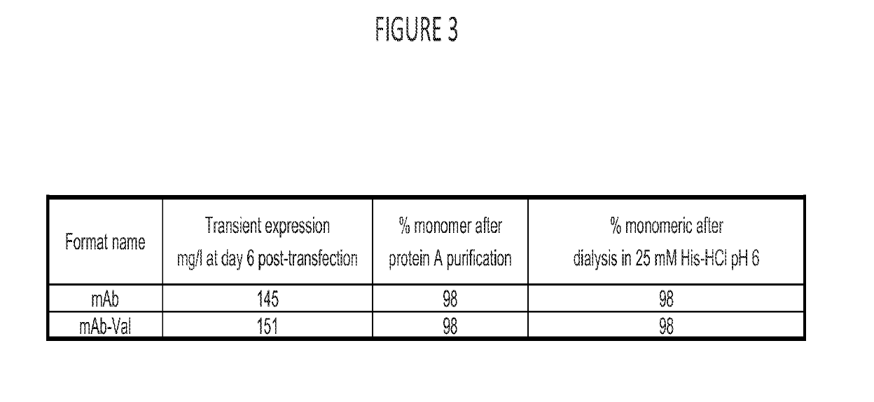

The culture medium was collected 6 days post-transfection and the two antibodies were purified by standard protein A affinity chromatography in accordance with the manufacturer's protocol (GE Healthcare, Piscataway, N.J.). The expression level as shown in FIG. 3 was 145 mg/L and 151 mg/L at day 6 post-transfection for mAb and FlexiMab, respectively.

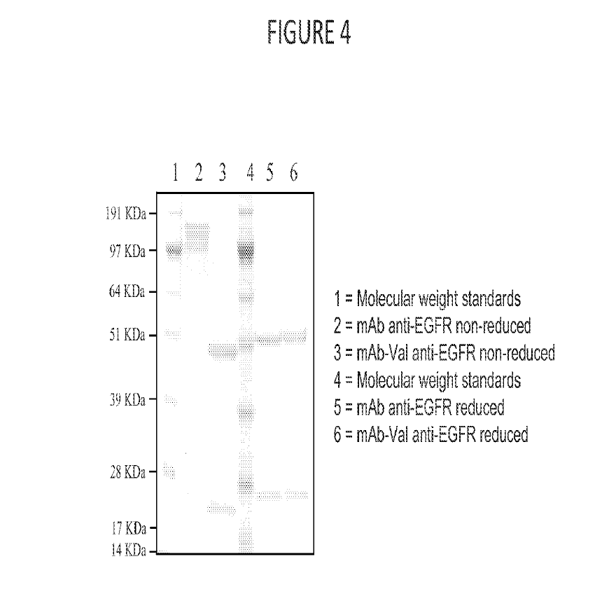

Total IgG expression was determined using a protein A binding assay. The protein A quantification method is as follow. The culture media was automatically loaded onto a protein A column using an HPLC system (Agilent 1100 Capillary LC System, Foster City, Calif.). Unbound material was washed with a solution of 100 mM sodium phosphate buffer at pH 6.8, and antibodies were eluted with 0.1% phosphoric acid, pH 1.8. The area corresponding to the eluted peak was integrated and the total antibody concentration was determined by comparing to an IgG standard. The concentrations of the purified antibodies were also determined by reading the absorbance at 280 nm using theoretically determined extinction coefficients. Analytical size-exclusion HPLC chromatography (SEC-HPLC, Agilent 1100 Capillary LC System) was used to determine the monomeric content of constructs. The wavelength was set to 280 nm and the experiments were carried out at 25° C. SEC-HPLC was carried out using TSK-GEL G3000SWXL column (Tosoh Bioscience LLC, Montgomeryville, Pa.), which separates globular proteins with molecular weight (MW) that range from approximately 10 to 500 KDa, with a buffer containing 100 mM sodium phosphate, pH 6.8, and at a flow rate of 1 mL/min. A low molecular weight gel filtration calibration kit from Bio-Rad (Hercules, Calif.) containing vitamin B12 (11,350 Da), equine myoglobin (17,000 Da), chicken ovalbumin (44,000 Da), bovine gamma-globulin (158,000 Da) and thyroglobulin (670,000 Da) was used as molecular mass standards. In addition, a highly purified 99% monomeric IgG was used as immunoglobulin molecular weight standard. As shown in FIG. 3, the monomeric content after protein A purification for mAb and FlexiMab was 98% and 98%, respectively. Subsequent to protein A purification the proteins were buffer exchanged into 25 mM Histidine-HCl, pH 6.0. The purity of the constructs was analyzed (1) by using analytical SEC-HPLC and as shown in FIG. 3, and by using (2) standard sodium dodecyl sulfate polyacrylamide gel electrophoresis (SDS-PAGE) under reducing and non-reducing conditions (FIG. 4). As shown in FIG. 4, in non-reducing conditions, lane 2, the conventional mAb runs as intact interchain disulphide-linked mAb, whereas in non-reducing conditions FlexiMab runs as two impendent chains (similarly to what expected for a reduced sample) heavy (upper band in FIG. 4 lane 3) and light chain (lower band in FIG. 4 lane 3). FIG. 4 lane 5 and line 6 show the heavy and light chains for mAb and FlexiMab in reducing conditions. As expected in reducing conditions there are two distinct protein bands, corresponding to the heavy and light chains, respectively. The molecular weight standards used in the SDS-PAGE analysis are schematically shown on the left of the SDS-PAGE image.

### Example 3—ELISA Assays for Determining Functional Binding of the FlexiMab to its Antigen (EGFR) and Comparison with the Conventional mAb

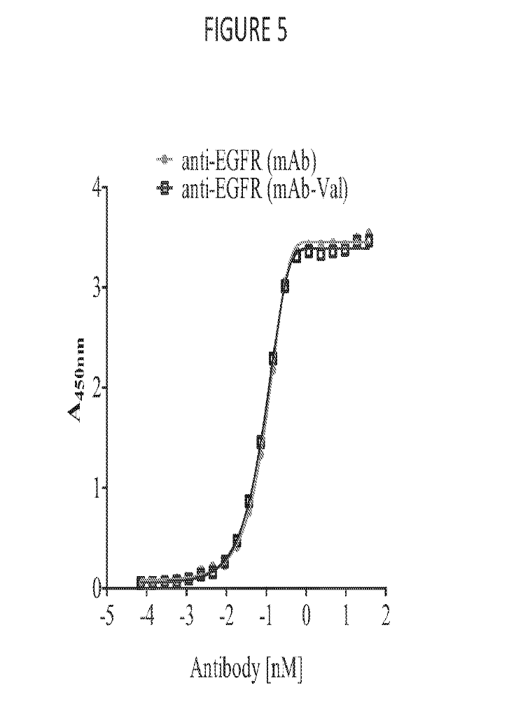

As shown in FIG. 5, FlexiMab is able to bind its antigen EGFR in an ELISA analysis comparable with its parental mAb. For the ELISA binding analysis, 2 ug/mL of antigen in 30 μL of PBS, pH 7.4 were coated on microtiter ELISA wells for 1 hour at room temperature. Antigen-coated wells were washed 3 times with PBS containing 0.1% (v/v) Tween-20 and blocked for 1 hour at room temperature with 3% BSA. Antibodies were serially diluted in 30 uL of blocking solution and were incubated for 2 hour at 37° C., followed by extensive washes with PBS containing 0.1% (v/v) Tween-20. Bound antibodies were detected by HRP-conjugated anti-human-kappa-antibody and visualized with 30 uL of 3,3″,5,5″-tetramethylbenzidine substrate (Pierce). The reaction was stopped by adding 30 uL of 0.18 M sulfuric acid (Pierce). The absorbance at 450 nm was measured using a microtiter ELISA plate reader. The resulting data were analyzed and plotted using Prism 5 software (Graph Pad, San Diego, Calif.).

### Example 4—FACS Assays for Determining Functional Binding of the FlexiMab to its Native Antigen Expressed on the Surface of A431 (Human Epithelial Carcinoma Cell Line) Cells and Comparison with the Conventional mAb

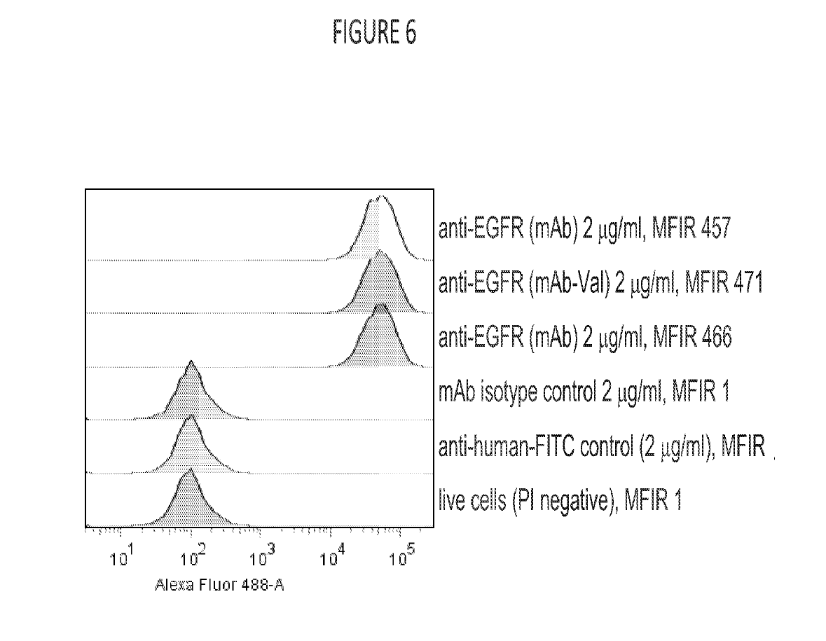

As shown in FIG. 6, FlexiMab is functionally able to bind its ligand EGFR expressed on the cell surface of A431 cells. As indicated in FIG. 6, the binding signal for FlexiMab and the conventional mAb is comparable. The mean fluorescence intensity values (MFIR) are schematically shown in the figure for FlexiMab, mAb and controls. MFRI values are comparable for FlexiMab and the conventional mAb. A431 cells were grown in F12-K Medium with 10% FBS and detached using Trypsin (0.25%) (Invitrogen, location?, USA). Cells were washed and resuspended in 3% BSA, in PBS. A total of 100 uL of cells at 1.5×10⁶ cells/mL were dispensed into a 96 well micro-plate. Cells were stained with primary antibody for 30 minutes, 4° C. Cells were then washed three times and stained with 2 ug/mL anti-human IgG-FITC for 30 min, 4° C., for detecting antibody. After washing with 3% BSA in PBS, cells were resuspended in wash buffer containing 2 ug/mL Propidium Iodide. Cell-associated fluorescence was analyzed using the LSR II flow cytometer (Bectin, Dickonson) and plotted using the program FlowJo.

### Example 5—Functional Stability of FlexiMab and mAb after Incubation at 37° C. in PBS for 5 Days

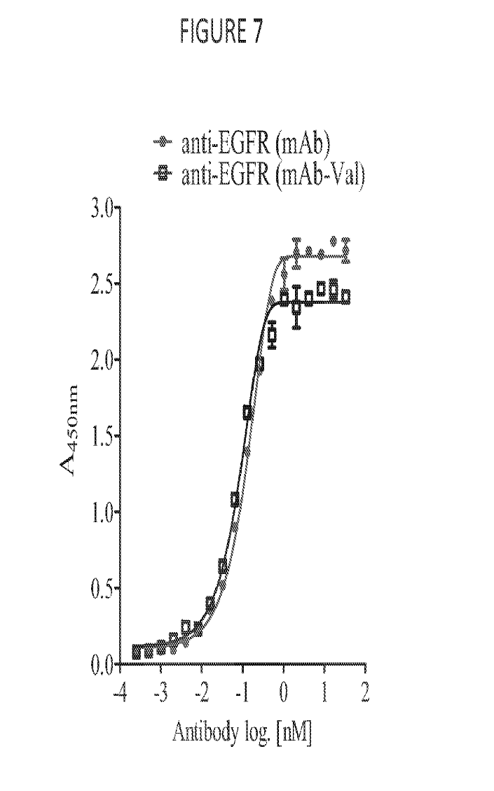

Assays, results for which are shown in FIG. 7, were carried out to determine if the FlexiMab retains binding activity to its antigen upon temperature induced experimental stress. The antibodies were incubated for 5 days in PBS at 37° C. Upon incubation, functional activity was determined using ELISA analysis as described in Example 4. As shown in FIG. 7, ELISA analysis shows that FlexiMab and mAb have comparable binding signals for EGFR, and indicates that FlexiMab is a stable molecule.

### Example 6—Functional Stability of FlexiMab and mAb after Incubation at 37° C. in Human Serum for 5 Days

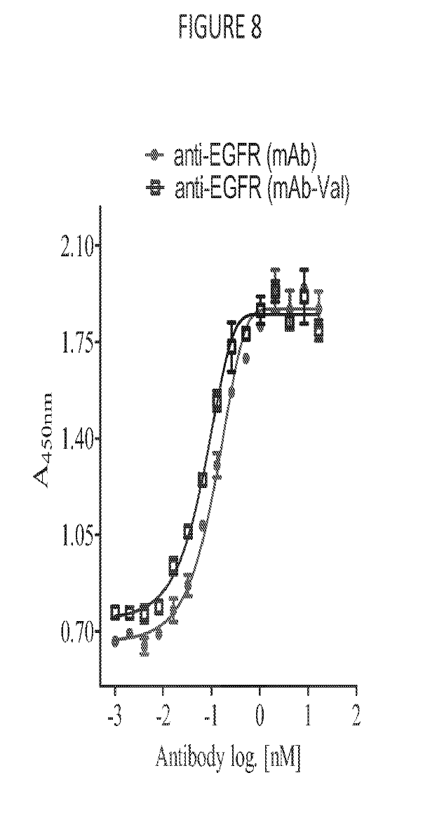

Results shown in FIG. 8 were obtained to determine if the FlexiMab retains binding activity to its antigen upon incubation for 5 days in total human serum. The antibodies were incubated for 5 days in total human serum at 37° C. Upon incubation, functional activity was determined using ELISA analysis as described in Example 4, except that the ELISA analysis was carried out with 5% final human serum concentration. As shown in FIG. 8, this analysis shows that FlexiMab and mAb have comparable binding signals for EGFR, and indicates that FlexiMab is a stable molecule and is not susceptible to protease degradation by serum proteases.

### Example 6—Kinetic Parameters of FlexiMab and mAb Analyzed Using BIAcore

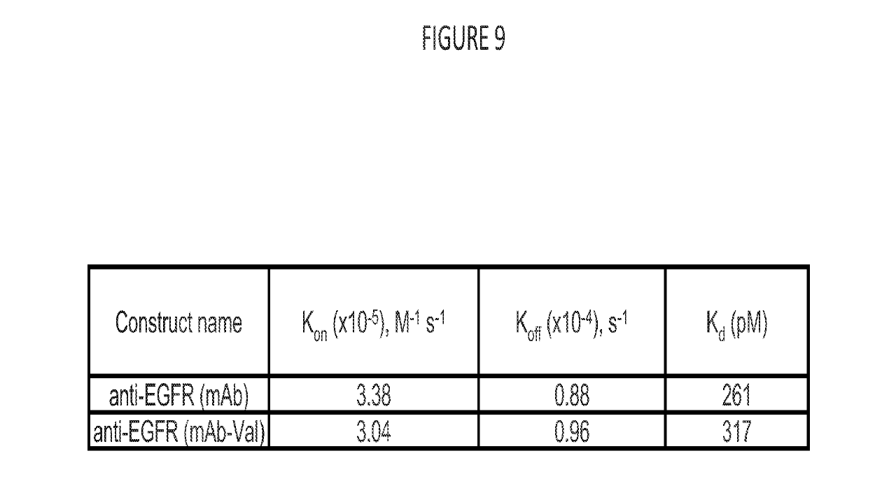

The results shown in FIG. 9 are BIAcore data carried out to determine the Kon, Koff and Kd of the FlexiMab and mAb. As shown in FIG. 9, FlexiMab and mAb exhibit similar kinetic parameters for EGFR ligand immobilized on the BIAcore sensor chip. BIAcore experiments were carried out using BIAcore 3000 instrument (Biacore International) and using standard protocols as supplied by the manufacturer. EGFR was coupled to a dextran matrix of a CM5 sensor chip (Pharmacia Biosensor) using a standard amine coupling kit. Excess reactive esters were quenched by injection of 70 μL of 1.0 M ethanolamine hydrochloride (pH 8.5). The antibodies were injected at a flow rate of 5 uL/min. Responses were analyzed using BIOEVALUATION software and Kd is shown in pico-molar units.

### Example 7—Inhibition of Cell Survival Mediated by FlexiMab and mAb

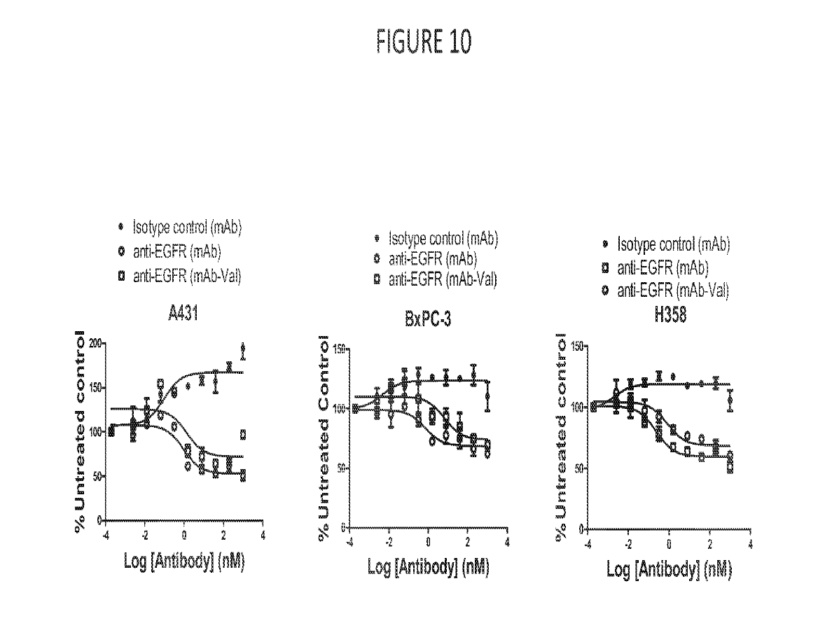

Results in FIG. 10 show inhibition of cancer cell survival by an anti-EGFR antibody (mAb) in its classic format and of the anti-EGFR FlexiMab antibody variant. A non-binding isotype control antibody was used in these experiments. Three different cell lines were used: A431 (human epidermal carcinoma cells), BxPC3 (human pancreatic cancer cells) and H358 (human non-small lung adenocarcinoma cells). All of these cell lines express EGFR on their cell surface. As shown in FIG. 10, FlexiMab inhibits cancer cell survival and its inhibitory activity compared well with its parental mAb control antibody. The cells were cultured in standard medium and plated to non-tissue culture treated plates to a density of 5K. Antibodies in serial dilution were added to the cells and incubated in standard cell culture incubators at 37° C. for 72 hours. A luminescent cell titer assay (CellTiter-Glo) was used, according to manufacturer's instructions, to determine the number of viable cells by quantization of the ATP present in cell lysates, which is an indicator of metabolically active cells.

### Example 8—Killing of Cancer Cells by FlexiMab and mAb Conjugated with an Anti-Human-Saporin Antibody

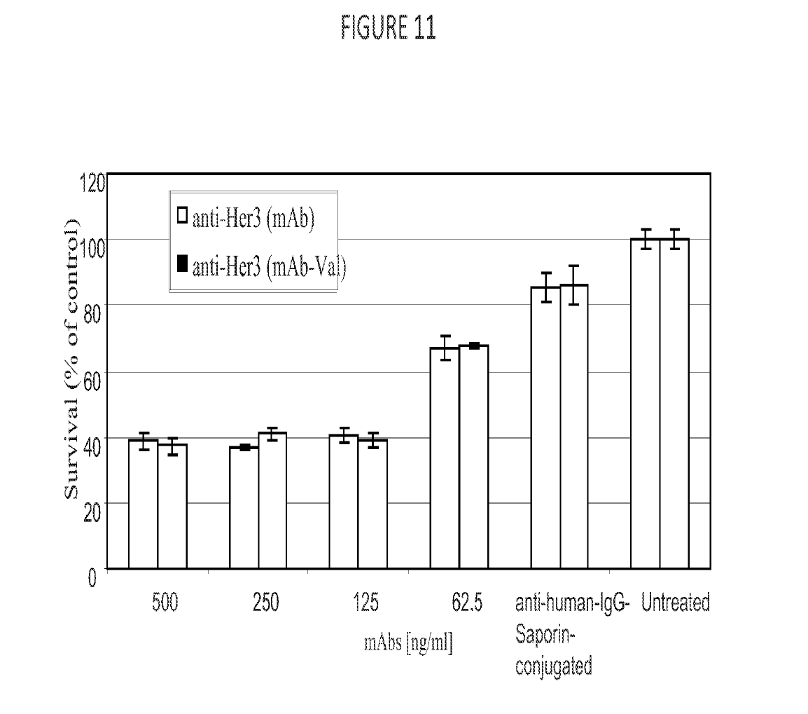

Results in FIG. 11 show killing of cancer cells SKBr3 (human mammary carcinoma cells) over-expressing Her3 on their cell surface. Specific FlexiMab and mAb used in this experiment are anti-Her3 specific antibodies. FlexiMab and mAb show similar in vitro cytotoxicity, indicating that both antibodies bind and internalize similarly to the Her3 receptor. These two antibodies were pre-complexed with an anti-human IgG-Saporin conjugate (Advanced Targeting Systems; San Diego). Upon receptor binding and internalization, Saporin will be released inside the cells. The release of Saporin into the cell will result in protein synthesis inhibition and cell death after approximately 72 hours. In these killing experiments negative control used were un-treated cells and cell-treated with anti-human-IgG-Saporin-conjugate only. As shown in FIG. 11, the FlexiMab-Saporin conjugated shows a dose-dependence killing of SKBr3 comparable to mAb-Saporin conjugated. SKBr3 cells were plated at 3000 cells/90 uL/well and incubated overnight. Antibodies-Saporin conjugated and controls dilutions as indicated in FIG. 11, were made in cell culture medium RPM11640 supplemented with 10% FBS, and 10 uL of each dilution was added to each well in triplicate. The plates were incubated for 72 hours. Cell viability was determined by using CellTiter-Glo Luminescent Cell Viability Assay (Promega, Madison Wis.). As instructed by manufacture, 100 μl of CellTiter-Glo reagent was added into each well and incubated at room temperature for 10 minutes with gentle shaking. The luminescence of each sample in the plates was measured in a plate-reading luminometer and the cytotoxicity was calculated by using the luminescence signal of untreated cells as control with 100% of cell survival. Data analysis was done with Prism software (Graphpad, San Diego).

### Example 9—Steady-State Kinetic Affinities (Kd) of FlexiMab and mAb for Fcgamma Receptors, FcgammaRIIIa Genotype Variant 158V and Genotype Variant 158F and FcRn, Analyzed Using BIAcore

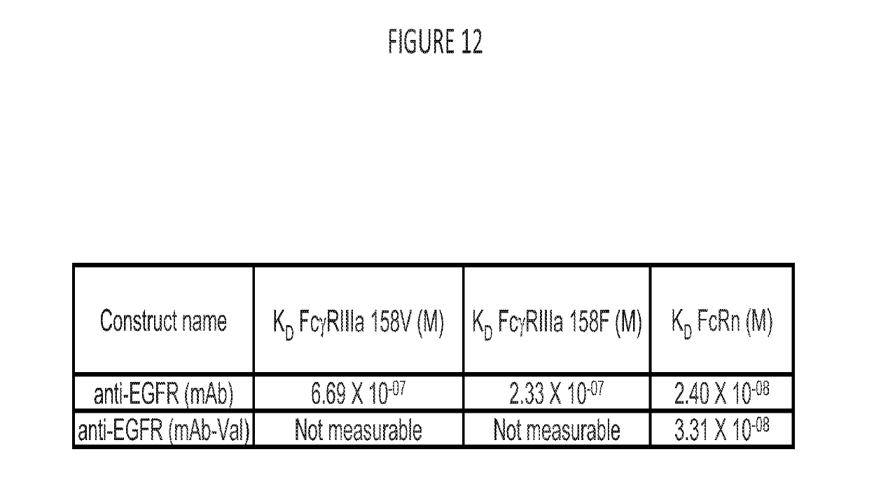

The example shown in FIG. 12 is BIAcore determined steady-state Kd in Molar units for FlexiMab and mAb. As shown in FIG. 12, FlexiMab and mAb exhibit similar Kd values for FcRn binding, implying comparable in vivo half-life for these two antibodies. A Kd for FcgammaRIIIa could not be measured for FlexiMab (158V and 158F genotypes). These results indicate that Fc-mediated antibody effector functions could be potentially abrogated in the FlexiMab. BIAcore experiments were carried out using BIAcore 3000 instrument (Biacore International) and using standard protocols as supplied by the manufacturer. FcgammaRIIIA (158V and 158F genotypes) were captured on the BIAcore sensor chip using anti-histidine chips (Pharmacia Biosensor) and using a standard protocol as supplied by the manufacturer. For the FcRn measurements, FcRn was directly immobilized onto the BIAcore sensor chip using standard amino coupling chemistry as described in Example 6. FlexiMab and mAb were flowed at a flow rate of 5 uL/min over the captured/immobilized receptors. Responses were analyzed using the BIOEVALUATION software.

### Example 10—Pharmacokinetics Analysis of FlexiMab and mAb

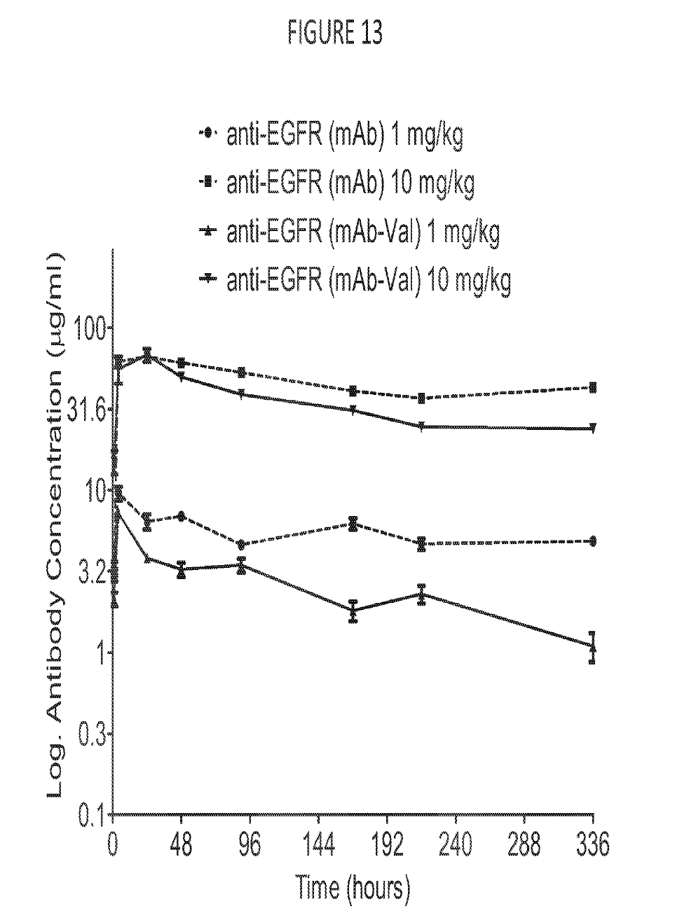

Results in FIG. 13 show antibody concentration, in a logrithimic representation, versus time of circulation in mouse for mAb and FlexiMab dosed at 1 mg/kg and 10 mg/kg. As shown in FIG. 13, mAb and FlexiMab have comparable in vivo half-life at high dose (10 mg/kg) and low dose (1 mg/kg). Pharmacokinetic analysis was carried out by intraperitoneal administration of 1 mg/kg and 10 mg/kg of mAb and FlexiMab antibodies into nude mice. The plasma concentrations of the antibodies were measured at 0, 1, 4, 24, 48, 90, 168, 216 and 336 hours post-dose and analyzed using ELISA methods as described in Examples 3, 5 and 6. An anti-human-kappa antibody was used for detection and standard curves for quantification were generated using known concentrations of mAb and FlexiMab.

### Example 11—Differential Scanning Calorimetry (DSC) Analysis of FlexiMab and mAb

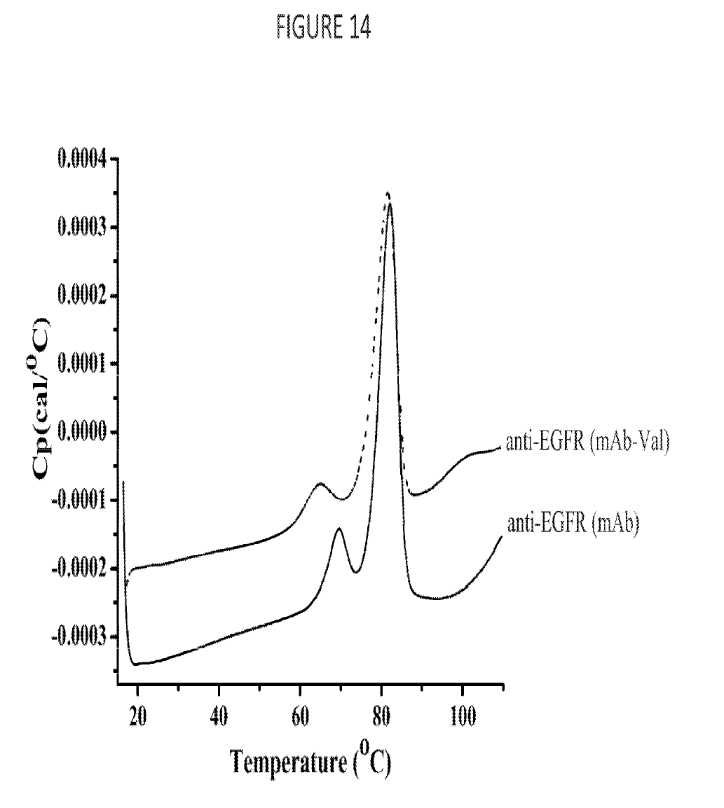

The example shown in FIG. 14 is a differential scanning calorimetry analysis of FlexiMab and mAb. Differential scanning calorimetry (DSC) experiments, as depicted in FIG. 14, at a heating rate of 1° C./min, were carried out using a Microcal VP-DSC ultrasensitive scanning microcalorimeter (Microcal, Northampton, Mass.). The thermograms showed in FIG. 14 are raw data baseline subtracted. DSC experiments were carried out in 25 mM Histidine-HCl, pH 6. All solutions and samples used for DSC were filtered using a 0.22 micron-filter and degassed prior to loading into the calorimeter. The two antibodies used for the DSC studies were >98% monomer as judged by analytical gel filtration chromatography (SEC-HPLC). For each set of measurements, at least four buffer-versus-baseline runs were first obtained. Immediately after, the buffer solution was removed from the sample cell and loaded with approximately 0.75 mL of sample at concentration of 1 mg/mL. For each measurement the reference cell was filled with matched sample buffer. In each sample-versus-buffer experiment, the corresponding buffer-versus-buffer baseline run was subtracted. The raw data were normalized for concentration and scan rate. Data analysis and deconvolution was carried out using the Origin™ DSC software provided by Microcal. FIG. 12 shows that the FlexiMab antibody showed two transition temperature peaks at 65° C. and at 82° C., respectively. Whereas, mAb antibody showed two transition temperature peaks at 69° C. and at 82° C., respectively. The DSC analysis showed that FlexiMab has a first transition temperature less than 4° C. when compared to mAb (first transition peak in FIG. 14), and has a comparable denaturation transition with the mAb for the second transition peak.

### Example 12—Monomeric Content of FlexiMab at 11 mg/mL Analyzed by Analytical Size-Exclusion Chromatography (SEC-HPLC)

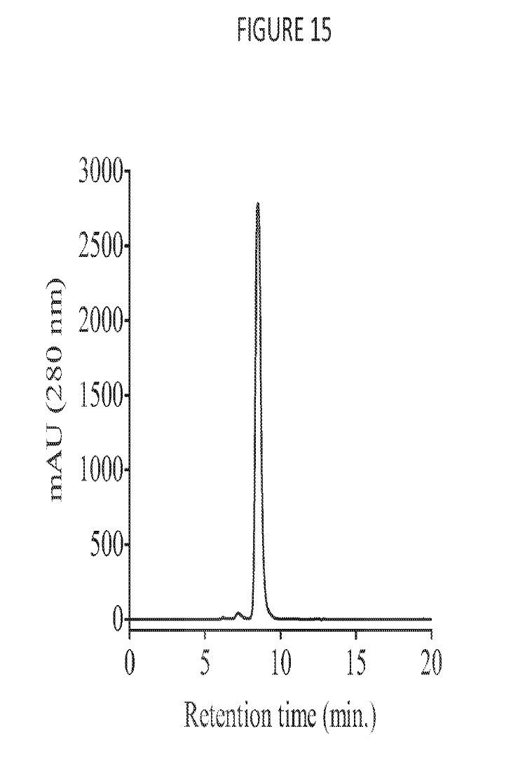

FIG. 15 shows results of an SEC-HPLC analysis of FlexiMab at 11 mg/mL in 25 mM Histidine-HCl pH 6. This example shows that FlexiMab is >98% monomeric at high concentration. The SEC-HPLC method performed is described in Example 2.

### Example 13—In Vivo Efficacy and Body Weight of FlexiMab and mAb Using A431 Xenograft

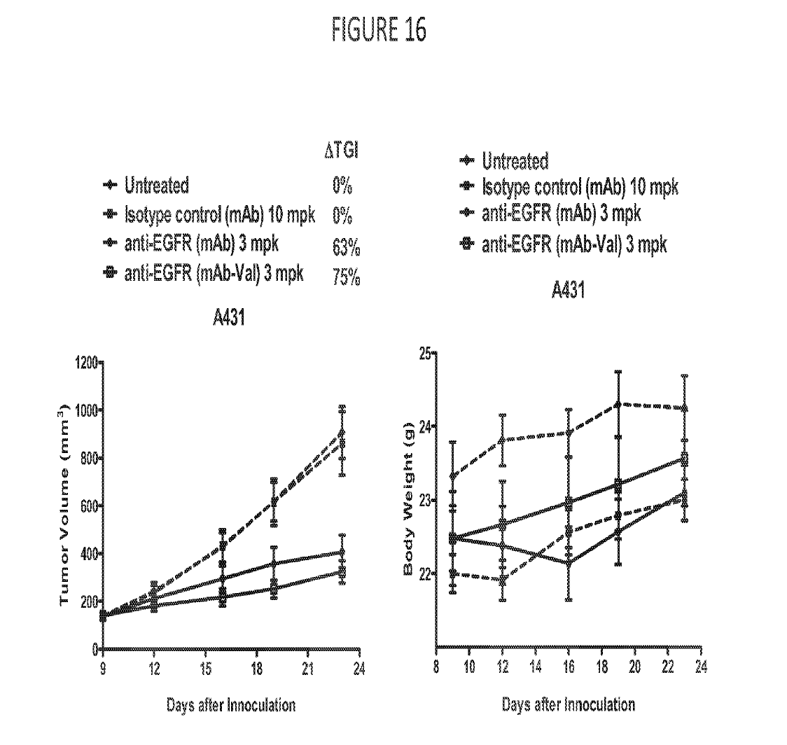

FIG. 16 shows tumor growth curves (left panel) and body weight curves (right panel) of A431 (human epithelial carcinoma cells) xenograft tumor in nude mice. In this example there were four mice groups, untreated control, and irrelevant isotype antibody control dosed at 10 mg/kg, mAb and FlexiMab groups dosed at 3 mg/kg. Nine nude mice were used in each group. The reported points (obtained at day 9, 12, 16, 19, and 23 post-inoculation) are average of tumor volume (left panel) and average of body weight (right panel) versus time of start of treatment. Mice were dosed with the antibodies when tumor reached an average volume size of 150-200 cubic millimeters. The tumor growth inhibition (DeltaTGI) is schematically reported (legend at left panel). The tumor growth inhibition is 63% and 75% for mAb and FlexiMab, respectively. These results indicate that FlexiMab is efficacious in vivo comparable (if not better) than its parental mAb. As also shown (left panel) there is no drastic difference in total body weight lost for mAb and FlexiMab.

### Example 14—Design of Cysteine Mutants in the Antibody CH1 Region 131-139 (EU Nomenclature) Using FlexiMab for Site-Specific Drug Conjugation

The example shown in FIG. 17 is a ribbon representation (left panel) of a FlexiMab with Fc and Fab domains schematically labeled. The black dots at the hinge and at the heavy and light chains represent valine substitutions of interchain cysteine amino acids (8 in total). Amino acids in the CH1 loop (expanded view, left panel) that were targeted for cysteine substitutions are labeled by amino acid and position in the ribbon representation. These residues are serine 131, serine 132, serine 134, threonine 135, serine 136, and threonine 139, and are in a CH1 structure region common to all four human immunoglobulin isotypes (IgG1, IgG2, IgG3 and IgG4; see Table 2). The 131-139 CH1 region is shown below in the sequence alignment as underlined bold text. The first underlined and embolded amino acid in the sequence alignment below corresponds to position 131 (serine in IgG1 and cysteine in IgG2, IgG3 and IgG4. The last underlined and embolded amino acid corresponds to position 139 (threonine in all four isotypes). Standard molecular biology techniques were used to generate single, double or triple FlexiMab cysteine variants.

| | | SEQ |
|---|---|---|
| | | ID |
| Subtype | Subequence | NO: |
| IgG1 | ASTKGPSVFPLAPSSKSTSGGTAALGCLVKDYFPEP | 11 |
| | VTVSWNSGALTSGVHTFPAVLQSS | |
| IgG2 | ASTKGPSVFPLAPCSRSTSGGTAALGCLVKDYFPEP | 12 |
| | VTVSWNSGALTSGVHTFPAVLQSS | |
| IgG3 | ASTKGPSVFPLAPCSRSTSESTAALGCLVKDYFPEP | 13 |
| | VTVSWNSGALTSGVHTFPAVLQSS | |
| IgG4 | ASTKGPSVFPLAPCSRSTSESTAALGCLVKDYFPEP | 14 |
| | VTVSWNSGALTSGVHTFPAVLQSS | |

### Example 15—Expression, Purification and Monomeric Content of FlexiMab Cysteine Variants

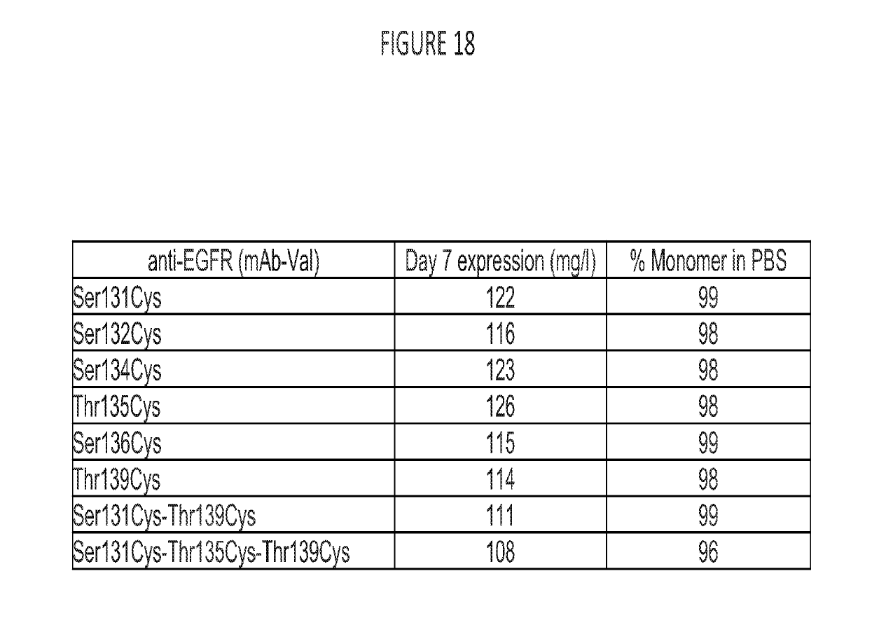

As shown in FIG. 18, eight (8) FlexiMab Cysteines variants were generated. Of these eight variants, six (6) are single cysteine variants (Ser131Cys, Ser132Cys, Ser134Cys, Thr135Cys, Ser136Cys and Thr139Cys), one is a double Cysteine variant (Ser131Cys-Thr139Cys) and one is a triple cysteine variant (Ser131Cys-Thr135Cys-Thr139Cys). The expression level as shown in FIG. 18 was ranging from 108 mg/L to 126 mg/L at day 7 post-transfection for all cysteine variants. This expression level is similar to the expected expression level of the FlexiMab antibody (FIG. 3, Example 2). Total IgG expression was determined using a protein A binding assay as described in Example 2. Analytical size-exclusion HPLC chromatography (SEC-HPLC, Agilent 1100 Capillary LC System) was used, as detailed in Example 2, to determine the monomeric content of the cysteine constructs in PBS buffer. As shown in FIG. 18, the monomeric content for FlexiMab cysteine mutants ranged from 96% to 99% monomer.

### Example 16—Site-Specific Conjugation, Using a Maleimide-PEG(2)-Biotin, of FlexiMab Cysteine Variants

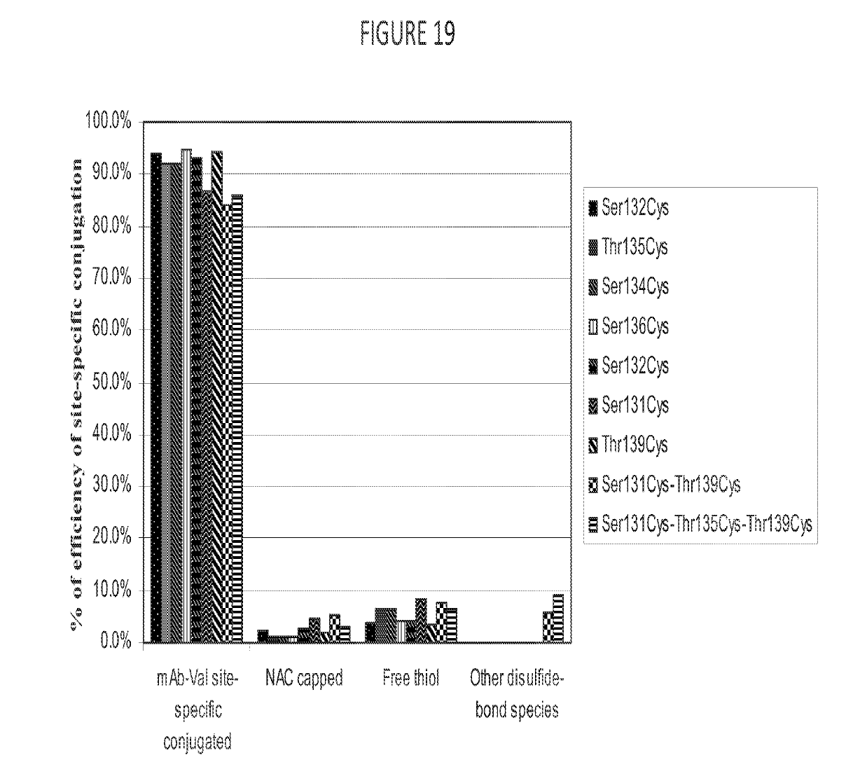

FIG. 19 shows the efficiency of site-specific conjugation for single, double and triple FlexiMab Cysteine variants. As shown in FIG. 19, high efficiency of site-specific conjugation is achieved using cysteine variants engineered using the FlexiMab backbone. Because FlexiMab does not have a native interchain cysteine at the hinge or at the heavy and light chains, the site-specific conjugation is efficient and there is no scrambling on interchain disulfide bonds between the native and the engineered cysteines. The antibody cysteine variants used for conjugation were dialyzed overnight in 4 liters of 0.1 M Na-phosphate, 0.15 M NaCl, 10 mM EDTA, pH 7.4. The antibodies were removed from the dialysis apparatus and filtered through a 0.2 μM syringe filter. Using sterile Eppendorf tubes, 1 mg of the respective antibody variant was mixed with 1.87 uL of 50 mM TCEP solution [Tris-(2-carboxyethyl)-phosphine); Pierce] and 10 μL of DTPA [Diethylenetriaminepentaacetic acid; Sigma-Aldrich] and incubated at 37° C. for 2 hours under constant rotation. After the incubation, the antibodies were incubated with a 3 molar excess of Maleimide-PEG(2)-Biotin (Pierce). This incubation was done by adding 13.14 uL of a 3.8 uM Maleimide-PEG(2)-Biotin (MW 525.62 Da; Pierce) solution and 78 uL of DMSO (dimethyl sulfoxide; Sigma-Aldrich) to the TCEP-reduced antibody cysteine variants. The mixture was incubated for 30 minutes at 4° C. The reaction was stopped by adding 2.5 uL of NAC [N-acetyl-L-cysteine; Sigma-Aldrich] and by mixing gently for 5 minutes. The conjugated antibodies were dialyzed overnight in 1×PBS pH 7.4 at 4° C. to remove the non-reacted Maleimide-PEG(2)-Biotin. Efficiency of conjugation was measured using standard mass spectrometry and peptide mapping as described in U.S. Patent Application Publication No. 2009092011 entitled “Cysteine engineered antibodies for site-specific conjugation.”

### Example 17—FlexiMab is O-Glycosylated at Threonine in Position 225 (EU Numbering) at Upper Region of Human IgG1 Hinge

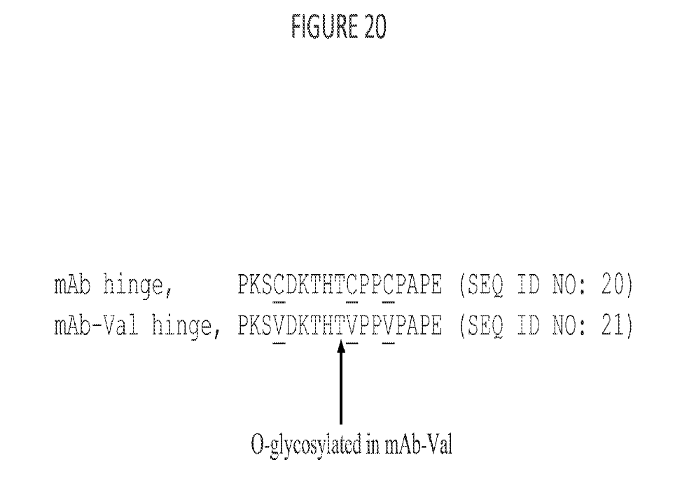

Shown in FIG. 20 is a sequence alignment of the upper human IgG1 hinge region for mAb and FlexiMab. In this sequence alignment the cysteines in mAb and the valine substitutions in FlexiMab are underlined. Intact mass and peptide mapping have shown that the threonine at position 225 (EU nomenclature), shown in FIG. 20 with an arrow, is modified post-translationally by a potential O-glycosylation. Standard mass spectrometry and peptide mapping techniques were used.

### Example 18—Examples of Embodiments

Provided hereafter are non-limiting examples of some embodiments.

A1. An antibody, comprising:
a heavy chain having no native interchain cysteine amino acids;
a light chain having no native interchain cysteine amino acids; and
no native interchain disulphide linkages between the heavy chain and the light chain.

A1.1. The antibody of embodiment A1, comprising no interchain disulphide linkages between the heavy chain and the light chain.

A1.2. An antibody, comprising:
a heavy chain having no interchain cysteine amino acids;
a light chain having no interchain cysteine amino acids; and
no interchain disulphide linkages between the heavy chain and the light chain.

A2. The antibody of any one of embodiments A1 to A1.2, comprising two heavy chains and two light chains.

A3. The antibody of any one of embodiments A1 to A2, which is a full-length antibody.

A4. The antibody of any one of embodiments A1 to A3, wherein the heavy chain is about 446 amino acids in length and the light chain is about 214 amino acids in length.

A4.1. An antibody comprising a heavy chain comprising the amino acid sequence of SEQ ID NO: 5, and a light chain comprising the amino acid sequence of SEQ ID NO: 9 or SEQ ID NO: 10, wherein each of the cysteines at positions 103, 109, and 112 in SEQ ID NO: 5, and the cysteine at position 105 in SEQ ID NO: 9 or the cysteine at position 102 in SEQ ID NO: 10, are substituted by an amino acid that is not cysteine, and wherein the antibody comprises no interchain cysteine amino acids and no interchain disulfide linkages.

A4.2. An antibody comprising a heavy chain comprising the amino acid sequence of SEQ ID NO: 5, wherein each of the cysteines at positions 103, 109, and 112 in SEQ ID NO: 5 are substituted by an amino acid that is not cysteine, and wherein the antibody comprises no interchain cysteine amino acids and no interchain disulfide linkages.

A4.3. An antibody comprising a heavy chain fragment comprising a portion of the amino acid sequence of SEQ ID NO: 5, provided that should the fragment include an amino acid at position(s) 103, 109, and/or 112 in SEQ ID NO: 5, the amino acid at each of the positions is not a cysteine, and wherein the antibody comprises no interchain cysteine amino acids and no interchain disulfide linkages.

A4.4. The antibody of embodiment A4.2 or A4.3, which comprises a light chain comprising the amino acid sequence of SEQ ID NO: 9 or SEQ ID NO: 10, wherein the cysteine at position 105 in SEQ ID NO: 9 or the cysteine at position 102 in SEQ ID NO: 10 is substituted by an amino acid that is not cysteine.

A4.5. The antibody of embodiment A4.2 or A4.3, which comprises a light chain fragment comprising a portion of the amino acid sequence of SEQ ID NO: 9 or SEQ ID NO: 10, provided that should the fragment include position 105 of SEQ ID NO: 9 or position 102 of SEQ ID NO: 10, the amino acid at that position is not a cysteine.

A4.6. An antibody comprising an amino acid sequence comprising 80% or more amino acid sequence identitiy to an antibody of any one of embodiments A4.1 to A4.5, wherein the antibody comprises no interchain cysteine amino acids and no interchain disulfide linkages.

A4.7. An antibody comprising a heavy chain comprising the amino acid sequence of SEQ ID NO: 6, and a light chain comprising the amino acid sequence of SEQ ID NO: 9 or SEQ ID NO: 10, wherein each of the cysteines at positions 14, 103, 106, and 109 in SEQ ID NO: 6, and the cysteine at position 105 in SEQ ID NO: 9 or the cysteine at position 102 in SEQ ID NO: 10, are substituted by an amino acid that is not cysteine, and wherein the antibody comprises no interchain cysteine amino acids and no interchain disulfide linkages.

A4.8. An antibody comprising a heavy chain comprising the amino acid sequence of SEQ ID NO: 6, wherein each of the cysteines at positions 14, 103, 106, and 109 in SEQ ID NO: 6 are substituted by an amino acid that is not cysteine, and wherein the antibody comprises no interchain cysteine amino acids and no interchain disulfide linkages.

A4.9. An antibody comprising a heavy chain fragment comprising a portion of the amino acid sequence of SEQ ID NO: 6, provided that should the fragment include an amino acid at position(s) 14, 103, 106, and/or 109 in SEQ ID NO: 6, the amino acid at each of the positions is not a cysteine, and wherein the antibody comprises no interchain cysteine amino acids and no interchain disulfide linkages.

A4.10. The antibody of embodiment A4.8 or A4.9, which comprises a light chain comprising the amino acid sequence of SEQ ID NO: 9 or SEQ ID NO: 10, wherein the cysteine at position 105 in SEQ ID NO: 9 or the cysteine at position 102 in SEQ ID NO: 10 is substituted by an amino acid that is not cysteine.

A4.11. The antibody of embodiment A4.8 or A4.9, which comprises a light chain fragment comprising a portion of the amino acid sequence of SEQ ID NO: 9 or SEQ ID NO: 10, provided that should the fragment include position 105 of SEQ ID NO: 9 or position 102 of SEQ ID NO: 10, the amino acid at that position is not a cysteine.

A4.12. An antibody comprising an amino acid sequence comprising 80% or more amino acid sequence identitiy to an antibody of any one of embodiments A4.7 to A4.11, wherein the antibody comprises no interchain cysteine amino acids and no interchain disulfide linkages.

A4.13. An antibody comprising a heavy chain comprising the amino acid sequence of SEQ ID NO: 7, and a light chain comprising the amino acid sequence of SEQ ID NO: 9 or SEQ ID NO: 10, wherein each of the cysteines at positions 14, 110, 113, 118 and 121 in SEQ ID NO: 7, and the cysteine at position 105 in SEQ ID NO: 9 or the cysteine at position 102 in SEQ ID NO: 10, are substituted by an amino acid that is not cysteine, and wherein the antibody comprises no interchain cysteine amino acids and no interchain disulfide linkages.

A4.14. An antibody comprising a heavy chain comprising the amino acid sequence of SEQ ID NO: 7, wherein each of the cysteines at positions 14, 110, 113, 118 and 121 in SEQ ID NO: 7 are substituted by an amino acid that is not cysteine, and wherein the antibody comprises no interchain cysteine amino acids and no interchain disulfide linkages.

A4.15. An antibody comprising a heavy chain fragment comprising a portion of the amino acid sequence of SEQ ID NO: 7, provided that should the fragment include an amino acid at position(s) 14, 110, 113, 118 and/or 121 in SEQ ID NO: 7, the amino acid at each of the positions is not a cysteine, and wherein the antibody comprises no interchain cysteine amino acids and no interchain disulfide linkages.

A4.16. The antibody of embodiment A4.14 or A4.15, which comprises a light chain comprising the amino acid sequence of SEQ ID NO: 9 or SEQ ID NO: 10, wherein the cysteine at position 105 in SEQ ID NO: 9 or the cysteine at position 102 in SEQ ID NO: 10 is substituted by an amino acid that is not cysteine.

A4.17. The antibody of embodiment A4.14 or A4.15, which comprises a light chain fragment comprising a portion of the amino acid sequence of SEQ ID NO: 9 or SEQ ID NO: 10, provided that should the fragment include position 105 of SEQ ID NO: 9 or position 102 of SEQ ID NO: 10, the amino acid at that position is not a cysteine.

A4.18. An antibody comprising an amino acid sequence comprising 80% or more amino acid sequence identitiy to an antibody of any one of embodiments A4.13 to A4.17, wherein the antibody comprises no interchain cysteine amino acids and no interchain disulfide linkages.

A4.19. An antibody comprising a heavy chain comprising the amino acid sequence of SEQ ID NO: 8, and a light chain comprising the amino acid sequence of SEQ ID NO: 9 or SEQ ID NO: 10, wherein each of the cysteines at positions 14, 106 and 109 in SEQ ID NO: 8, and the cysteine at position 105 in SEQ ID NO: 9 or the cysteine at position 102 in SEQ ID NO: 10, are substituted by an amino acid that is not cysteine, and wherein the antibody comprises no interchain cysteine amino acids and no interchain disulfide linkages.

A4.20. An antibody comprising a heavy chain comprising the amino acid sequence of SEQ ID NO: 8, wherein each of the cysteines at positions 14, 106 and 109 in SEQ ID NO: 8 are substituted by an amino acid that is not cysteine, and wherein the antibody comprises no interchain cysteine amino acids and no interchain disulfide linkages.

A4.21. An antibody comprising a heavy chain fragment comprising a portion of the amino acid sequence of SEQ ID NO: 8, provided that should the fragment include an amino acid at position(s) 14, 106 and/or 109 in SEQ ID NO: 8, the amino acid at each of the positions is not a cysteine, and wherein the antibody comprises no interchain cysteine amino acids and no interchain disulfide linkages.

A4.22. The antibody of embodiment A4.20 or A4.21, which comprises a light chain comprising the amino acid sequence of SEQ ID NO: 9 or SEQ ID NO: 10, wherein the cysteine at position 105 in SEQ ID NO: 9 or the cysteine at position 102 in SEQ ID NO: 10 is substituted by an amino acid that is not cysteine.

A4.23. The antibody of embodiment A4.20 or A4.21, which comprises a light chain fragment comprising a portion of the amino acid sequence of SEQ ID NO: 9 or SEQ ID NO: 10, provided that should the fragment include position 105 of SEQ ID NO: 9 or position 102 of SEQ ID NO: 10, the amino acid at that position is not a cysteine.

A4.24. An antibody comprising an amino acid sequence comprising 80% or more amino acid sequence identitiy to an antibody of any one of embodiments A4.19 to A4.23, wherein the antibody comprises no interchain cysteine amino acids and no interchain disulfide linkages.

A5. The antibody of any one of embodiments A1 to A4.24, which is a human antibody.

A6. The antibody of any one of embodiments A1 to A5, which is a humanized antibody.

A7. An antibody comprising a heavy chain and a light chain, wherein the amino acid sequence of the light chain is about 80% or more identical to SEQ ID NO: 3 and the amino acid sequence of the heavy chain is about 80% or more identical to SEQ ID NO: 4, and wherein the antibody comprises no interchain cysteine amino acids and no interchain disulfide linkages.

A8. The antibody of any one of embodiments A1 to A7, wherein the native interchain cysteine amino acids are replaced by amino acids having no thiol moiety.

A9. The antibody of embodiment A8, wherein an amino acid at positions in Table 1 each are replaced by an amino acid having no thiol moiety.

A10. The antibody of embodiment A8 or A9, wherein one or more of the native interchain cysteine amino acids are replaced by valine.

A11. The antibody of embodiment A10, wherein all of the native interchain cysteine amino acids are replaced by valine.

A12. The antibody of any one of embodiments A1 to A11, comprising a glycosylation site not present in an antibody counterpart having a native interchain cysteine amino acid.

A13. The antibody of any one of embodiments A1 to A12, which comprises an Fc region or fragment thereof.

A14. The antibody of embodiment A13, comprising one or more cysteine replacements of non-cysteine surface amino acids in the CH1 domain, CH2 domain, or CH3 domain, or combination thereof, of the antibody.

A15. The antibody of embodiment A14, comprising about 2 to about 40 of the cysteine replacements of the non-cysteine surface amino acids.

A16. The antibody of embodiment A14 or A15, which comprises about 2 to about 40 free thiols.

A17. The antibody of any one of embodiments A14 to A16, which is a human or humanized antibody.

A18. The antibody of embodiment A17, wherein the one or more cysteine replacements are at one or more of positions shown in Table 2.

A19. The antibody of embodiment A18, wherein the one or more cysteine replacements are at one or more of serine 131, serine 132, serine 134, threonine 135, serine 136 and threonine 139 of an IgG1 antibody, or counterpart position in an IgG2, IgG3 or IgG4 antibody.

A20. The antibody of embodiment A19, wherein the one or more cysteine replacements are at one or more of serine 131, threonine 135 and threonine 139 of IgG1 antibody, or counterpart position in an IgG2, IgG3 or IgG4 antibody.

A21. The antibody of any one of embodiments A14 to A20, wherein the one or more cysteine replacements are at one or more of positions shown in Table 3.

A22. The antibody of any one of embodiments A1 to A21, which has a stability of about 70% or more compared to an antibody counterpart containing all native interchain cysteines.

A23. The antibody of embodiment A22, wherein the stability is in vitro stability.

A24. The antibody of embodiment A23, wherein the stability is in serum at about 37 degrees Celsius for 5 days or more.

A25. The antibody of embodiment A23, wherein the stability is determined by calorimetry.

A26. The antibody of embodiment A22, wherein the stability is in vivo stability.

A27. The antibody of embodiment A25, wherein the stability is in an animal for 14 days or more.

A28. The antibody of any one of embodiments A1 to A27, which has a specific binding activity of about 70% or more compared to an antibody counterpart containing all native interchain cysteines.

A29. The antibody of embodiment A28, wherein the specific binding activity is in vitro.

A30. The antibody of embodiment A29, wherein the specific binding activity is quantified by an in vitro homogeneous assay or an in vitro heterogeneous assay.

A31. The antibody of embodiment A28, wherein the specific binding activity is in vivo.

A32. The antibody of embodiment A31, wherein the specific binding activity is determined in situ.

A33. The antibody of any one of embodiments A1 to A32, which has a cell proliferation inhibition activity of about 70% or more compared to an antibody counterpart containing all native interchain cysteines.

A34. The antibody of embodiment A33, wherein the cell proliferation inhibition activity is inhibition of proliferation of cancer cells.

A35. The antibody of embodiment A33 or A34, wherein the activity is in vitro.

A36. The antibody of embodiment A33 or A34, wherein the activity is in vivo.

A37. The antibody of any one of embodiments A1 to A36, which is about 90% or more monomeric.

A38. The antibody of embodiment A37, which is 90% or more monomeric in vitro.

A39. The antibody of any one of embodiments A1 to A38, which does not bind detectably to a human leukocyte receptor.

A40. The antibody of embodiment A39, wherein the human leukocyte receptor is a Fc gamma RIII receptor.

A41. The antibody of any one of embodiments A1 to A40, which binds to a neonatal Fc receptor with about 80% or more of the binding affinity compared to an antibody counterpart containing all native interchain cysteines.

A42. The antibody of any one of embodiments A1 to A32, which specifically binds to a cell surface molecule.

A43. The antibody of embodiment A42, wherein the cell surface molecule is internalized in a cell.

A44. The antibody of embodiment A43, wherein the cell surface molecule is a cell surface receptor.

A45. The antibody of embodiment A44, wherein the cell surface receptor comprises a protein kinase domain.

A46. The antibody of embodiment A45, wherein the cell surface receptor is an epidermal growth factor receptor (EGFR) protein tyrosine kinase.

A47. The antibody of embodiment A46, wherein the cell surface receptor is a HER3 protein tyrosine kinase.

B1. The antibody of any one of embodiments A1 to A41, which is an antibody conjugate in association with one or more heterologous molecules.

B2. The antibody of embodiment B1, wherein the antibody conjugate is about 80% or more of antibody products in a conjugation reaction product mixture.

B3. The antibody of embodiment B1 or B2, comprising one or more cysteine replacements of non-cysteine surface amino acids in the CH1 domain, CH2 domain, or CH3 domain, or combination thereof, of the antibody, wherein the one or more heterologous molecules are linked to the one or more cysteine replacements.

B4. The antibody of any one of embodiments B1 to B3, wherein the one or more heterologous molecules comprise a therapeutic agent.

B5. The antibody of embodiment B4, wherein the therapeutic agent comprises a toxin.

B6. The antibody of any one of embodiments B1 to B3, wherein the one or more heterologous molecules comprise a diagnostic agent.

B7. The antibody of embodiment B6, wherein the diagnostic agent comprises an imaging agent.

B8. The antibody of embodiment B7, wherein the diagnostic agent comprises a detectable label.

B9. The antibody of any one of embodiments B1 to B8, wherein the one or more heterologous molecules are linked to the antibody via a linker.

C1. The antibody of any one of embodiments A1 to A47, which is part of an antibody homomultimer conjugate.

C2. The antibody of embodiment C1, comprising one or more cysteine replacements of non-cysteine surface amino acids in the CH1 domain, CH2 domain, or CH3 domain, or combination thereof, of the antibody, wherein antibodies in the antibody homomultimer conjugate include a disulfide linkage between the one or more cysteine replacements.

D1. A nucleic acid comprising a nucleotide sequence that encodes an antibody of any one of embodiments A1 to A47.

D2. A cell comprising a nucleic acid of embodiment D1.

D3. An expression system comprising a nucleic acid of embodiment D1.

D4. An organism comprising a nucleic acid of embodiment D1.

D5. An organism comprising an expression system of embodiment D3.

E1. A process, comprising:
expressing an antibody in an expression system of embodiment D3, and
isolating the antibody, thereby producing an isolated antibody.

E2. The process of embodiment E1, comprising conjugating the isolated antibody with a heterologous molecule, thereby preparing an antibody conjugate.

E3. The process of embodiment E1, comprising conjugating the isolated antibody with other isolated antibody, thereby preparing an antibody multimer.

F1. A method, comprising:
contacting an antibody of any one of embodiments A1 to C2 with a biological sample; and
detecting the presence, absence or amount of antibody specifically bound to a molecule of interest in the biological sample.

F2. The method of embodiment F1, comprising linking the antibody to a solid support.

F3. A method, comprising:
administering an antibody of any one of embodiments A1 to C2 to cells; and
detecting the presence, absence or amount of antibody in a location of the cells.

F4. A method, comprising:
administering an antibody of any one of embodiments A1 to C2 to a subject; and
detecting the presence, absence or amount of antibody in a tissue of the subject.

F5. A method, comprising:
administering an antibody of any one of embodiments A1 to C2 to cells; and
detecting the presence, absence or amount of a biological effect associated with the administration of the antibody to the cells.

F6. A method, comprising:
administering an antibody of any one of embodiments A1 to C2 to a subject; and
detecting the presence, absence or amount of a biological effect in the subject associated with the administration of the antibody.

F7. The method of embodiment F5 or F6, wherein the biological effect is cell proliferation inhibition.

F8. A method, comprising:
administering an antibody of any one of embodiments A1 to C2 to a subject; and
monitoring the condition of the subject.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, these modifications and improvements are within the scope and spirit of the technology.

The technology illustratively described herein suitably may be practiced in the absence of any element(s) not specifically disclosed herein. Thus, for example, the term “comprising” in each instance encompasses the terms “consisting essentially of” or “consisting of.” The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. Use of the term “about” at the beginning of a string of values modifies each of the values (i.e., “about 1, 2 and 3” refers to about 1, about 2 and about 3). In certain instances units and formatting are expressed in HyperText Markup Language (HTML) format, which can be translated to another conventional format by those skilled in the art (e.g., “.sup.” refers to superscript formatting). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

1.-6. (canceled)

7. An antibody comprising a heavy chain comprising the amino acid sequence of SEQ ID NO: 5, and a light chain comprising the amino acid sequence of SEQ ID NO: 9 or SEQ ID NO: 10, wherein each of the cysteines at positions 103, 109, and 112 in SEQ ID NO: 5, and the cysteine at position 105 in SEQ ID NO: 9 or the cysteine at position 102 in SEQ ID NO: 10, are substituted by an amino acid that is not cysteine, and wherein the antibody comprises no interchain cysteine amino acids and no interchain disulfide linkages.

8.-11. (canceled)

12. An antibody comprising an amino acid sequence comprising 80% or more amino acid sequence identity to an antibody of claim 7, wherein the antibody comprises no interchain cysteine amino acids and no interchain disulfide linkages.

13. An antibody comprising a heavy chain comprising the amino acid sequence of SEQ ID NO: 6, and a light chain comprising the amino acid sequence of SEQ ID NO: 9 or SEQ ID NO: 10, wherein each of the cysteines at positions 14, 103, 106, and 109 in SEQ ID NO: 6, and the cysteine at position 105 in SEQ ID NO: 9 or the cysteine at position 102 in SEQ ID NO: 10, are substituted by an amino acid that is not cysteine, and wherein the antibody comprises no interchain cysteine amino acids and no interchain disulfide linkages.

14.-17. (canceled)

18. An antibody comprising an amino acid sequence comprising 80% or more amino acid sequence identity to an antibody of claim 13, wherein the antibody comprises no interchain cysteine amino acids and no interchain disulfide linkages.

19. An antibody comprising a heavy chain comprising the amino acid sequence of SEQ ID NO: 7, and a light chain comprising the amino acid sequence of SEQ ID NO: 9 or SEQ ID NO: 10, wherein each of the cysteines at positions 14, 110, 113, 118 and 121 in SEQ ID NO: 7, and the cysteine at position 105 in SEQ ID NO: 9 or the cysteine at position 102 in SEQ ID NO: 10, are substituted by an amino acid that is not cysteine, and wherein the antibody comprises no interchain cysteine amino acids and no interchain disulfide linkages.

20.-23. (canceled)

24. An antibody comprising an amino acid sequence comprising 80% or more amino acid sequence identity to an antibody of claim 19, wherein the antibody comprises no interchain cysteine amino acids and no interchain disulfide linkages.

25.-39. (canceled)

40. The antibody of claim 7, comprising one or more cysteine replacements of non-cysteine surface amino acids in the CH1 domain, CH2 domain, or CH3 domain, or combination thereof, of the antibody.

41.-43. (canceled)

44. The antibody of claim 7, wherein the one or more cysteine replacements are at one or more of positions shown in Table 2.

45. (canceled)

46. (canceled)

47. The antibody of claim 7, wherein the one or more cysteine replacements are at one or more of positions shown in Table 3.

48. The antibody of claim 7, which has a stability of about 70% or more compared to an antibody counterpart containing all native interchain cysteines.

49.-53. (canceled)

54. The antibody of claim 7, which has a specific binding activity of about 70% or more compared to an antibody counterpart containing all native interchain cysteines.

55.-58. (canceled)

59. The antibody of claim 7, which has a cell proliferation inhibition activity of about 70% or more compared to an antibody counterpart containing all native interchain cysteines.

60.-62. (canceled)

63. The antibody of claim 7, which is about 90% or more monomeric.

64.-73. (canceled)

74. The antibody of claim 7, which is an antibody conjugate in association with one or more heterologous molecules.

75. The antibody of claim 74, wherein the antibody conjugate is about 80% or more of antibody products in a conjugation reaction product mixture.

76. The antibody of claim 74, comprising one or more cysteine replacements of non-cysteine surface amino acids in the CH1 domain, CH2 domain, or CH3 domain, or combination thereof, of the antibody, wherein the one or more heterologous molecules are linked to the one or more cysteine replacements.

77.-97. (canceled)

98. A method, comprising:
administering an antibody of claim 7 to a subject; and
detecting the presence, absence or amount of a biological effect in the subject associated with the administration of the antibody.

99. The method of claim 98, wherein the biological effect is cell proliferation inhibition.

100. (canceled)


SEQUENCE LISTING (21 sequences)

SEQ ID NO: 1 - protein, 214 aa, Artificial Sequence
  Description of Artificial Sequence Synthetic polypeptide
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, Artificial Sequence
  Description of Artificial Sequence Synthetic polypeptide
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, Artificial Sequence
  Description of Artificial Sequence Synthetic polypeptide
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEV

SEQ ID NO: 4 - protein, 449 aa, Artificial Sequence
  Description of Artificial Sequence Synthetic polypeptide
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTVPPVPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 7 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFNWYVDGVEVHNAKTKPREEQYNSTYRVVSVLTVLHQDWLNGKEYKCKVSNKALPAPIEKTISKTKGQPREPQVYTLPPSREEMTKNQVSLTCLVKGFYPSDIAVEWESSGQPENNYKTTPPVLDSDGSFFLYSKLTVDKSRWQQGNIFSCSVMHEALHNHFTQKSLSLSPGK

SEQ ID NO: 8 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 105 aa, Homo sapiens [sequence omitted]

SEQ ID NO: 10 - protein, 103 aa, Homo sapiens
KAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 60 aa, Homo sapiens
ASTKGPSVFPLAPSSKSTSGGTAALGCLVKDYFPEPVTVSWNSGALTSGVHTFPAVLQSS

SEQ ID NO: 12 - protein, 60 aa, Homo sapiens
ASTKGPSVFPLAPCSRSTSGGTAALGCLVKDYFPEPVTVSWNSGALTSGVHTFPAVLQSS

SEQ ID NO: 13 - protein, 60 aa, Homo sapiens
ASTKGPSVFPLAPCSRSTSESTAALGCLVKDYFPEPVTVSWNSGALTSGVHTFPAVLQSS

SEQ ID NO: 14 - protein, 60 aa, Homo sapiens; identical to SEQ ID NO: 13

SEQ ID NO: 15 - DNA, 642 bp, Artificial Sequence
  Description of Artificial Sequence Synthetic polynucleotide
gacatccagatgacccagagccccagcagcctgagcgccagcgtgggcgacagagtgaccatcacctgccaggccagccaggacatcagcaactacctgaactggtatcagcagaagcccggcaaggcccccaagctgctgatctacgacgccagcaacctggagacaggcgtgcccagcagattcagcggcagcggctccggcaccgacttcaccttcaccatcagcagcctccagcccgaggatatcgccacctacttttgccagcacttcgaccacctgcccctggcctttggcggcggaacaaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 16 - DNA, 1347 bp, Artificial Sequence
  Description of Artificial Sequence Synthetic polynucleotide
caggtgcagctccaggagagcggccctggcctggtgaagcccagcgagacactgagcctgacctgcaccgtgtccggcggcagcgtgtccagcggcgactactactggacctggatcagacagagccccggcaagggcctggagtggatcggccacatctactacagcggcaacaccaactacaaccccagcctgaagtccagactgaccatcagcatcgacaccagcaagacccagttcagcctgaagctgtccagcgtgacagccgccgacaccgccatctactactgcgtgagagacagagtgaccggcgctttcgacatctggggccagggcaccatggtgaccgtgtccagcgcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggtaaa

SEQ ID NO: 17 - DNA, 642 bp, Artificial Sequence
  Description of Artificial Sequence Synthetic polynucleotide
gacatccagatgacccagagccccagcagcctgagcgccagcgtgggcgacagagtgaccatcacctgccaggccagccaggacatcagcaactacctgaactggtatcagcagaagcccggcaaggcccccaagctgctgatctacgacgccagcaacctggagacaggcgtgcccagcagattcagcggcagcggctccggcaccgacttcaccttcaccatcagcagcctccagcccgaggatatcgccacctacttttgccagcacttcgaccacctgcccctggcctttggcggcggaacaaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagaggtc

SEQ ID NO: 18 - DNA, 1347 bp, Artificial Sequence
  Description of Artificial Sequence Synthetic polynucleotide
caggtgcagctccaggagagcggccctggcctggtgaagcccagcgagacactgagcctgacctgcaccgtgtccggcggcagcgtgtccagcggcgactactactggacctggatcagacagagccccggcaagggcctggagtggatcggccacatctactacagcggcaacaccaactacaaccccagcctgaagtccagactgaccatcagcatcgacaccagcaagacccagttcagcctgaagctgtccagcgtgacagccgccgacaccgccatctactactgcgtgagagacagagtgaccggcgctttcgacatctggggccagggcaccatggtgaccgtgtccagcgcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcgtctgacaaaactcacacagtcccaccggtcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggtaaa

SEQ ID NO: 19 - protein, 6 aa, Artificial Sequence
  Description of Artificial Sequence Synthetic 6xHis tag
HHHHHH

SEQ ID NO: 20 - protein, 17 aa, Homo sapiens
PKSCDKTHTCPPCPAPE

SEQ ID NO: 21 - protein, 17 aa, Artificial Sequence
  Description of Artificial Sequence Synthetic peptide
PKSVDKTHTVPPVPAPE